(19) United States
(12) Patent Application Publication
(10) Pub. No.: US 2024/0295812 A1
(43) Pub. Date: Sep. 5, 2024

(54) RESIST COMPOSITION, METHOD FOR FORMING RESIST PATTERN, METHOD FOR PRODUCING COMPOUNDS, INTERMEDIATE, AND COMPOUNDS

(71) Applicant: TOKYO OHKA KOGYO CO., LTD., Kawasaki-shi (JP)

(72) Inventors: Junichi Miyakawa, Kawasaki-shi (JP); Hiroki Kato, Kawasaki-shi (JP); KhanhTin Nguyen, Kawasaki-shi (JP); Takuya Ikeda, Kawasaki-shi (JP); Seiji Todoroki, Kawasaki-shi (JP); Tetsuya Matsushita, Kawasaki-shi (JP)

(21) Appl. No.: 18/566,338

(22) PCT Filed: Jun. 15, 2022
(86) PCT No.: PCT/JP2022/023904

§ 371 Date: Dec. 1, 2023

(30) Foreign Application Priority Data
Jun. 15, 2021 (JP) 2021-099364
Jun. 15, 2021 (JP) 2021-099674
Jun. 6, 2022 (JP) 2022-091800

(51) Int. Cl. G03F 7/004; C07C 25/18; C07C 211/63; C07C 303/22; C07C 309/12; C07C 381/12; C07D 333/76

(52) U.S. Cl. CPC G03F 7/0045; C07C 25/18; C07C 211/63; C07C 303/22; C07C 309/12; C07C 381/12; C07D 333/76

(57) ABSTRACT

A resin component (A1) and a compound (B0) represented by General Formula (b0) in which X⁰ represents a bromine atom or an iodine atom, R^{m} represents a hydroxy group, nb1 represents an integer of 1 to 5, and nb2 represents an integer of 0 to 4, 1≤nb1+nb2≤5 is satisfied, Yb⁰ represents a divalent linking group or a single bond, Vb⁰ represents a single bond, an alkylene group, or a fluorinated alkylene group, R⁰ represents a hydrogen atom, a fluorinated alkyl group having 1 to 5 carbon atoms, or a fluorine atom, and Rb¹ to Rb¹⁵ each independently represents a hydrogen atom or a substituent, and at least two of Rb¹ to Rb⁵ represent a fluorine atom or at least one of Rb¹ to Rb⁵ represents a perfluoroalkyl group

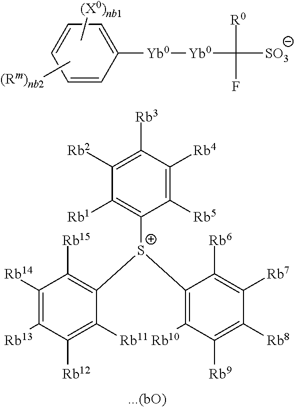

### TECHNICAL FIELD

The present invention relates to a resist composition, a method for forming a resist pattern, a method for producing compounds, an intermediate, and compounds.

Priority is claimed on Japanese Patent Application Nos. 2021-099364 and 2021-099674 filed Jun. 15, 2021, and Japanese Patent Application No. 2022-091800, filed Jun. 6, 2022, the contents of which are incorporated herein by reference.

### BACKGROUND ART

In recent years, in the manufacture of semiconductor elements and liquid crystal display elements, advances in lithography technologies have led to rapid progress in the field of pattern miniaturization. These pattern miniaturization techniques typically involve shortening the wavelength (increasing the energy) of the exposure light source.

Resist materials require lithography characteristics such as a high resolution that enables reproduction of patterns with minute dimensions, and a high level of sensitivity to these kinds of exposure light sources.

As a resist material that satisfies these requirements, a chemically amplified resist composition containing a base material component whose solubility in a developing solution is changed due to an action of an acid and an acid generator component that generates an acid upon light exposure has been used.

In the chemically amplified resist composition, a resin having a plurality of constitutional units is typically used in order to improve lithography characteristics and the like.

Further, in the formation of a resist pattern, the behavior of an acid generated from an acid generator component upon light exposure serves as an element that greatly affects the lithographic characteristics.

Various kinds of acid generators have been suggested as the acid generator used in a chemically amplified resist composition. Examples of such known acid generators include an onium salt-based acid generator such as an iodonium salt or a sulfonium salt, an oxime sulfonate-based acid generator, a diazomethane-based acid generator, a nitrobenzyl sulfonate-based acid generator, an imino sulfonate-based acid generator, and a disulfone-based acid generator.

For example, Patent Document 1 discloses a resist composition that employs, as an acid generator, a compound in which an electron-withdrawing group is introduced into a meta position of a sulfonium cation.

Further, with the further progress of the lithography technology, the expansion of application fields, and the like, a wide variety of acid generators have been developed for improving lithography characteristics. In addition, there is a demand for a production method that enables the acid generator to be obtained with a high yield.

For example, Patent Document 2 discloses a method for producing a second ammonium salt compound produced by reacting a first ammonium salt compound with a nitrogen-containing compound having a lone electron pair, in which the first ammonium salt compound contains a primary, secondary, or tertiary first ammonium cation, and a conjugate acid of the nitrogen-containing compound has an acid dissociation constant (pKa) greater than that of the first ammonium cation, and a method for producing a compound, including a step of performing salt exchange between the ammonium salt compound produced by the production method and a sulfonium cation or an iodonium cation having hydrophobicity higher than that of the conjugate acid of the nitrogen-containing compound. Further, in the examples of Patent Document 1, a method for producing a compound that contains a —SO₂-containing cyclic group having relatively high hydrophilicity in an anion moiety is disclosed. According to the method of producing a compound, Patent Document 1 discloses that an acid generator having less impurities can be obtained with a high yield.

### CITATION LIST

### Patent Document

### [Patent Document 1]

Japanese Unexamined Patent Application, First Publication No. 2017-15777

### [Patent Document 2]

Japanese Unexamined Patent Application, First Publication No. 2014-15433

### SUMMARY OF INVENTION

### Technical Problem

With the further progress of the lithography technology and resist pattern fining, there is a demand for a resist composition in which all the sensitivity, the roughness, and the pattern shape are satisfactory. In the resist composition of the related art, the sensitivity, the roughness, and the pattern shape are in a trade-off relationship, and there is a problem in that improvement of any one of the characteristics leads to degradation of other characteristics.

Further, in order to further improve lithography characteristics, various research has been conducted on the structure of the anion moiety of an onium salt-based acid generator. For example, an onium salt-based acid generator that has, in the anion moiety, a benzene ring containing an iodine atom or a bromine atom and has relatively high hydrophobicity has been developed. In an onium salt-based acid generator in which such an anion moiety has a specific structure, the yield is not sufficient in a case where the method for producing a compound of the related art as described in Patent Document 1 is used, and thus an optimal production method for such a desired structure has been required.

The present invention has been made in consideration of the above-described circumstances, and an object thereof is to provide a compound useful as an acid generator for a resist composition, an acid generator obtained by using the compound, a resist composition containing the acid generator, and a method for forming a resist pattern using the resist composition.

Further, the present invention has been made in consideration of the above-described circumstances, and an object thereof is to provide a method for producing a compound, in which a compound useful as an acid generator for a resist composition is obtained with a high yield, an intermediate of the compound, and a compound used by the method for producing the compound.

### Solution to Problem

In order to achieve the above-described object, the present invention employs the following configurations.

That is, according to a first aspect of the present invention, there is provided a resist composition which generates an acid upon light exposure and whose solubility in a developing solution is changed due to an action of the acid, the resist composition including a resin component (A1) whose solubility in a developing solution is changed due to the action of the acid, and an acid generator component (B) which generates an acid upon light exposure, in which the acid generator component (B) contains a compound (B0) represented by General Formula (b0).

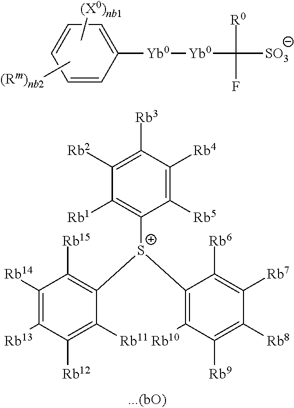

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, Yb° represents a divalent linking group or a single bond, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group.]

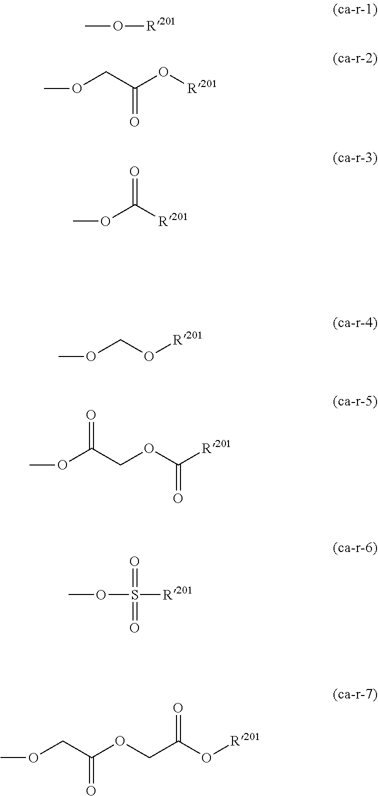

R′²⁰¹'s each independently represent a hydrogen atom, a cyclic group which may have a substituent, a chain-like alkyl group which may have a substituent, or a chain-like alkenyl group which may have a substituent.]

According to a second aspect of the present invention, there is provided a method for forming a resist pattern, including a step of forming a resist film on a support using the resist composition according to the first aspect, a step of exposing the resist film to light, and a step of developing the resist film exposed to light to form a resist pattern.

According to a third aspect of the present invention, there is provided a compound represented by General Formula (b0).

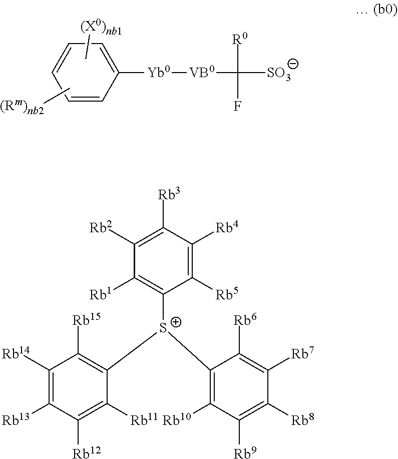

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, Yb⁰ represents a divalent linking group or a single bond, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group.]

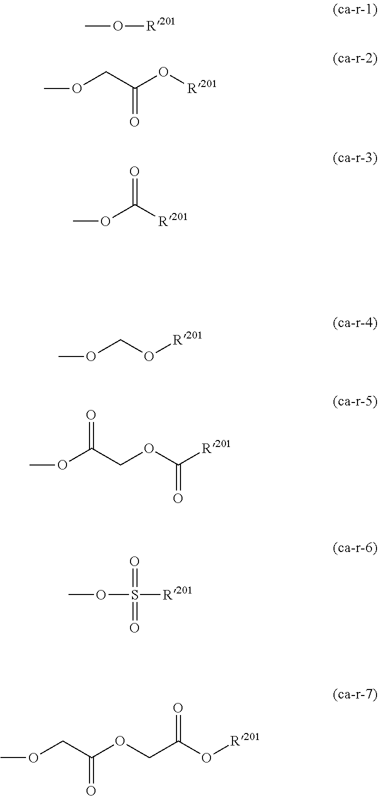

[In the formulae, R′²⁰¹'s each independently represent a hydrogen atom, a cyclic group which may have a substituent, a chain-like alkyl group which may have a substituent, or a chain-like alkenyl group which may have a substituent.]

According to a fourth aspect of the present invention, there is provided an acid generator containing the compound according to the third aspect described above.

According to a fifth aspect of the present invention, there is provided a method for producing a compound, including a step of performing a condensation reaction on a compound represented by General Formula (C-1) and a compound represented by General Formula (C-2) to obtain a compound (B0p) represented by General Formula (b0-p) and a step of performing an ion exchange reaction on the compound (B0p) and a compound represented by General Formula (C-3) to obtain a compound (b0′) represented by General Formula (b0′).

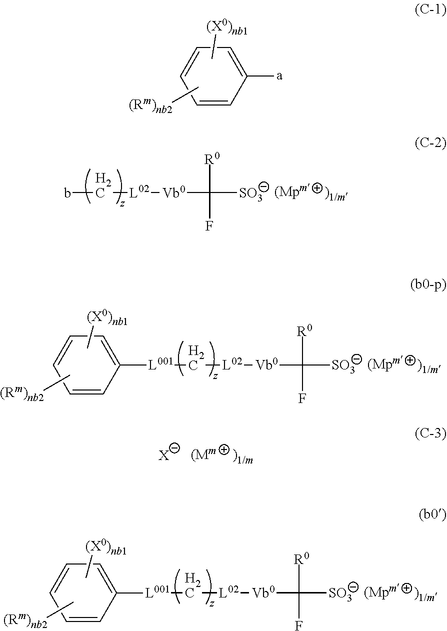

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, one of a and b represents a hydroxy group, and the other represents a carboxy group, z represents an integer of 0 to 10, L⁰² represents a single bond, an alkylene group, —O—, —CO—, —OCO—, —COO—, —SO₂—, —N(R^{a})—C(═O)—, —N(R^{a})—, —C(R^{a})(R′)—N(R^{a}), —C(R^{a})(N(R^{a})(R^{a}))—, or —C(═O)—N(R^{a})—, R^{a}'s each independently represent a hydrogen atom or an alkyl group, Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater, L⁰⁰¹ represents an ester bond [—C(═O)—O— or —O—C(═O)—], X⁻ represents a counter anion, M^{m+} * represents an m-valent organic cation, and m represents an integer of 1 or greater.]

According to a sixth aspect of the present invention, there is provided an intermediate used in the method for producing a compound according to the first aspect of the present invention, which is represented by General Formula (b0-p).

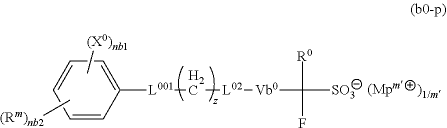

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰⁰¹ represents an ester bond [—C(═O)—O— or —O—C(═O)—], z represents an integer of 0 to 10, L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—, R^{a}'s each independently represent a hydrogen atom or an alkyl group, Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater.]

According to a seventh aspect of the present invention, there is provided a compound represented by General Formula (b0-p-1).

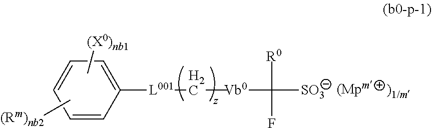

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰⁰¹ represents an ester bond [—C(═O)—O— or —O—C(═O)—], z represents an integer of 0 to 10, Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater.]

### Advantageous Effects of Invention

According to the present invention, it is possible to provide a compound useful as an acid generator for a resist composition, an acid generator obtained by using the compound, a resist composition containing the acid generator, and a method for forming a resist pattern using the resist composition.

Further, according to the present invention, it is possible to obtain a compound useful as an acid generator for a resist composition with a high yield.

### DESCRIPTION OF EMBODIMENTS

In the present description and the scope of the present patent claims, the term “aliphatic” is a relative concept used in relation to the term “aromatic”, and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid decomposable group” indicates a group having acid decomposability in which at least a part of a bond in the structure of the acid decomposable group can be cleaved due to the action of an acid.

Examples of the acid decomposable group whose polarity is increased due to the action of an acid include groups which are decomposed due to the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved due to the action of an acid and a group (ii) in which some bonds are cleaved due to the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, relatively, the solubility in a developing solution is changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, those having a molecular weight of 500 or greater and less than 4000 are typically used. Hereinafter, the term “low-molecular-weight compound” denotes a non-polymer having a molecular weight of 500 or greater and less than 4000. As the polymer, those having a molecular weight of 1000 or greater are typically used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{ax}) that substitutes the hydrogen atom bonded to the carbon atom at the α-position is an atom other than the hydrogen atom or a group. Further, the acrylic acid ester includes itaconic acid diester in which the substituent (R^{ax}) has been substituted with a substituent having an ester bond and α-hydroxyacryl ester in which the substituent (R^{ax}) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes the hydrogen atom at the α-position of hydroxystyrene include those for R^{ax}.

In the present specification and the scope of the present patent claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition According to First Aspect of Present Invention)

The resist composition according to the first aspect of the present invention is a resist composition that generates an acid upon light exposure and whose solubility in a developing solution is changed by the action of an acid.

Such a resist composition contains a base material component (A) (hereinafter, also referred to as “component (A)”) that exhibits changed solubility in a developing solution due to the action of an acid, and an acid generator component (B) that generates an acid upon light exposure (hereinafter, also referred to as “component (B)”).

In a case where a resist film is formed using the resist composition of the present embodiment and the formed resist film is subjected to selective light exposure, an acid is generated from the component (B) at an exposed portion of the resist film, and the solubility of the component (A) in a developing solution is not changed at an unexposed portion of the resist film while the solubility of the component (A) in a developing solution is changed due to the action of the acid, and thus a difference in solubility in a developing solution occurs between the exposed portion and the unexposed portion. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

In the present specification, a resist composition which forms a positive-tone resist pattern by dissolving and removing the exposed portion of the resist film is referred to as a positive-tone resist composition, and a resist composition which forms a negative-tone resist pattern by dissolving and removing the unexposed portion of the resist film is referred to as a negative-tone resist composition. The resist composition of the present embodiment may be a positive-tone resist composition or a negative-tone resist composition. Further, the resist composition of the present embodiment may be used in an alkali developing process using an alkali developing solution in the developing treatment in a case of forming a resist pattern or may be used in a solvent developing process using a developing solution containing an organic solvent (organic developing solution) in the developing treatment.

### <Component (A)>

In the resist composition according to the present embodiment, the component (A) contains a resin component (A1) (hereinafter, also referred to as “component (A1)”) whose solubility in a developing solution is changed due to the action of an acid. Since the polarity of the base material component before and after the light exposure is changed by using the component (A1), an excellent development contrast can be obtained not only in an alkali developing process but also in a solvent developing process.

As the component (A), another polymer compound and/or a low-molecular-weight compound may be used in combination with the component (A1).

In a case of applying an alkali developing process, the base material component having the component (A1) is insoluble in an alkali developing solution before light exposure, but in a case where an acid is generated from the component (B) upon light exposure, the action of this acid causes an increase in the polarity of the base material component, thereby increasing the solubility of the component (A1) in an alkali developing solution. Therefore, in a case where selective light exposure is performed on a resist film formed by coating a support with the resist composition in the formation of a resist pattern, the exposed portion of the resist film is changed from being insoluble to being soluble in an alkali developing solution, whereas the unexposed portion of the resist film remains insoluble in an alkali developing solution, and thus a positive-tone resist pattern is formed by performing alkali development.

On the other hand, in a case of a solvent developing process, the base material component containing the component (A1) exhibits high solubility in an organic developing solution prior to exposure, and in a case where an acid is generated from the component (B) upon light exposure, the polarity of the component (A1) is increased by the action of the generated acid, thereby decreasing the solubility of the component (A1) in an organic developing solution. Therefore, in a case where selective light exposure is performed on a resist film formed by coating a support with the resist composition in the formation of a resist pattern, the exposed portion of the resist film is changed from being soluble to being insoluble in an organic developing solution, whereas the unexposed portion of the resist film remains soluble in an organic developing solution. Therefore, a negative-tone resist pattern is formed by performing development using an organic developing solution so that a contrast is imparted between the exposed portion and the unexposed portion.

In the resist composition according to the present embodiment, the component (A) may be used alone or a combination of two or more kinds thereof may be used.

### In Regard to Component (A1)

The component (A1) is a resin component whose solubility in a developing solution is changed due to the action of an acid.

As the component (A1), those having a constitutional unit (a1) containing an acid decomposable group whose polarity is increased by the action of an acid are preferable.

Further, the component (A1) may have other constitutional units as necessary in addition to the constitutional unit (a1).

<<Constitutional Unit (a1)>>

The constitutional unit (a1) is a constitutional unit that contains an acid decomposable group whose polarity is increased due to the action of an acid.

Examples of the acid dissociable group are the same as those which have been suggested as the acid dissociable groups of the base resin for a chemically amplified resist composition.

Specific examples of the acid dissociable group of the base resin for a chemically amplified resist composition include “acetal type acid dissociable group”, “tertiary alkyl ester type acid dissociable group”, and “tertiary alkyloxycarbonyl acid dissociable group” described below.

### Acetal Type Acid Dissociable Group:

Examples of the acid dissociable group that protects a carboxy group or a hydroxyl group in the polar groups include an acid dissociable group represented by Formula (a1-r-1) (hereinafter, also referred to as “acetal type acid dissociable group”).

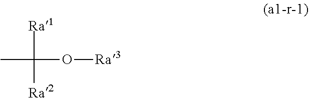

[In the formula, Ra′¹ and Ra′² represent a hydrogen atom or an alkyl group. Ra′³ represents a hydrocarbon group, and Ra′³ may be bonded to any one of Ra′¹ or Ra′² to form a ring.]

In Formula (a1-r-1), it is preferable that at least one of Ra′¹ and Ra′² represent a hydrogen atom and more preferable that both Ra′¹ and Ra′² represent a hydrogen atom. In a case where Ra′¹ or Ra′² represents an alkyl group, examples of the alkyl group include the same alkyl groups which are given as examples of the substituent which may be bonded to the carbon atom at the α-position in the description on α-substituted acrylic acid ester above. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group obtained by removing one hydrogen atom from the above-described aromatic hydrocarbon ring or aromatic heterocyclic ring (an aryl group or a heteroaryl group); a group obtained by removing one hydrogen atom from an aromatic compound having two or more aromatic rings (for example, biphenyl or fluorene); and a group obtained by substituting one hydrogen atom of the above-described aromatic hydrocarbon ring or aromatic heterocyclic ring with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may have a substituent. Examples of the substituent include —R^{P1}, —R^{P2}—O—R^{p1}, —R^{P2}—CO—R^{p1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P2} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. The aliphatic cyclic hydrocarbon group may have one or more of a single kind of substituents or one or more of each of plural kinds of the substituents.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.02,6] decanyl group, a tricyclo[3.3.1.13,7]decanyl group, a tetracyclo[6.2.1.13,6.02,7] dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

In a case where Ra′³ is bonded to any one of Ra′¹ or Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester Type Acid Dissociable Group:

Examples of the acid dissociable group that protects a carboxy group among the polar groups include an acid dissociable group represented by General Formula (a1-r-2).

Among examples of the acid dissociable group represented by Formula (a1-r-2), a group formed of an alkyl group is referred to as “tertiary alkyl ester type acid dissociable group” for convenience.

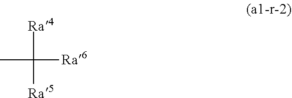

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ and Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent independent hydrocarbon groups that are not bonded to each other, suitable examples thereof include a group represented by Formula (a1-r2-4).

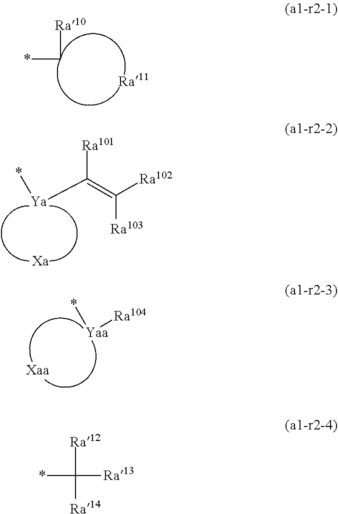

[In Formula (a1-r2-1), Ra′¹⁰ represents a linear or branched alkyl group having 1 to 12 carbon atoms, in which some carbon atoms may be substituted with a halogen atom or a hetero atom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a monovalent chain-like saturated hydrocarbon group having 1 to 10 carbon atoms. Some or all hydrogen atoms in this chain-like saturated hydrocarbon group may be substituted. Ra′¹⁴ represents a hydrocarbon group which may have a substituent. * represents a bonding site.]

In Formula (a1-r2-1), Ra′¹⁰ represents a linear or branched alkyl group having 1 to 12 carbon atoms, in which some carbon atoms may be substituted with a halogen atom or a hetero atom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include the same groups as those for Ra′³ described above.

The alkyl group as Ra′¹⁰ may be partially substituted with a halogen atom or a hetero atom-containing group. For example, some hydrogen atoms constituting the alkyl group may be substituted with a halogen atom or a hetero atom-containing group. Further, some carbon atoms (methylene group or the like) constituting the alkyl group may be substituted with a hetero atom-containing group.

Examples of the hetero atoms here include an oxygen atom, a nitrogen atom, and a sulfur atom. Examples of the hetero atom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups which are given as examples of the aliphatic hydrocarbon group (alicyclic hydrocarbon group) which is a monocyclic group or a polycyclic group as Ra′³ in Formula (a1-r-1). Among these, a monocyclic alicyclic hydrocarbon group is preferable, specifically, a cyclopentyl group or a cyclohexyl group is more preferable, and a cyclopentyl group is still more preferable.

In Formula (a1-r2-2), examples of the cyclic hydrocarbon group that is formed by Xa together with Ya include a group formed by further removing one or more hydrogen atoms from the cyclic monovalent hydrocarbon group (an aliphatic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2] octanyl group, a tricyclo[5.2.1.02,6] decanyl group, a tricyclo[3.3.1.13,7]decanyl group, a tetracyclo[6.2.1.13,6.02,7] dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same as those for Ra^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), preferred examples of the aliphatic cyclic group that is formed by Xaa together with Yaa include those given as examples of the aliphatic hydrocarbon group which is a monocyclic group or a polycyclic group as Ra′³ in Formula (a1-r-1).

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be included in Ra¹⁰⁴ in Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In Formula (a1-r2-4), Ra′¹² and Ra′¹³ each independently represent a monovalent chain-like saturated hydrocarbon group having 1 to 10 carbon atoms. Examples of the chain-like monovalent saturated hydrocarbon group having 1 to 10 carbon atoms as Ra′¹² and Ra′¹³ include those given as examples of the chain-like monovalent saturated hydrocarbon group having 1 to 10 carbon atoms as Ra¹⁰¹ to Ra¹⁰³. Some or all hydrogen atoms in this chain-like saturated hydrocarbon group may be substituted.

Among the examples, Ra′¹² and Ra′¹³ represent preferably an alkyl group having 1 to 5 carbon atoms, more preferably a methyl group or an ethyl group, and still more preferably a methyl group.

In a case where the chain-like saturated hydrocarbon group represented by Ra′¹² and Ra′¹³ is substituted, examples of the substituent are those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same groups as those for the substituent which may be included in Ra′⁰⁴.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

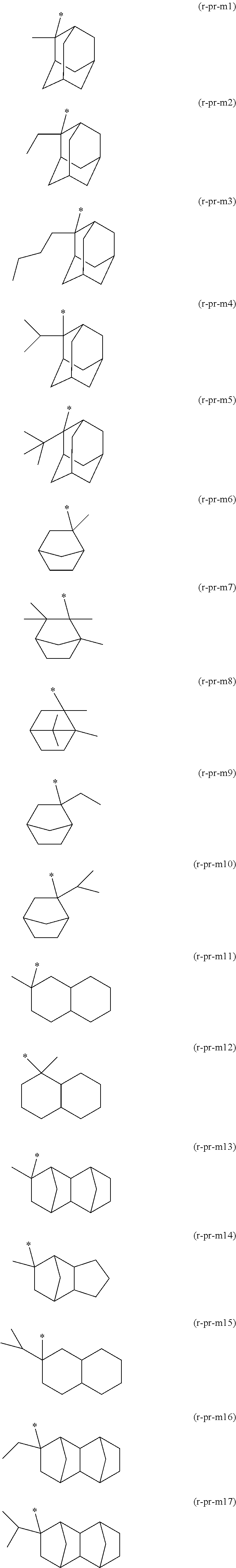

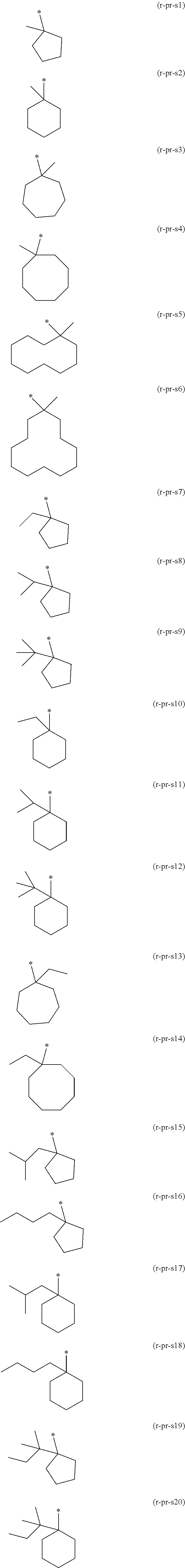

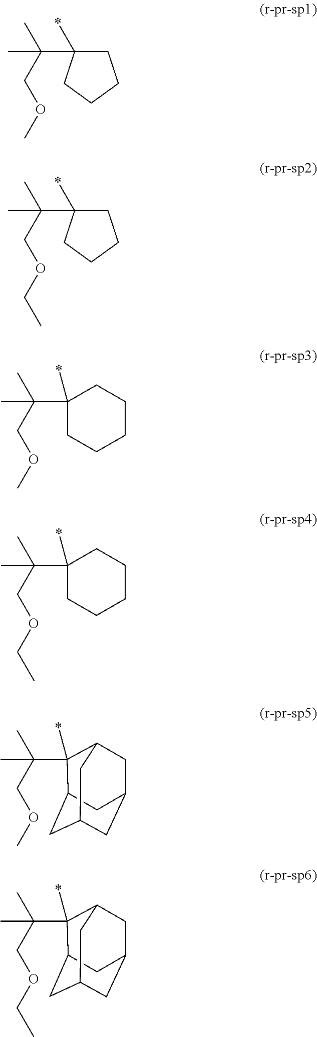

Specific examples of the group represented by Formula (a1-r2-2) are shown below.

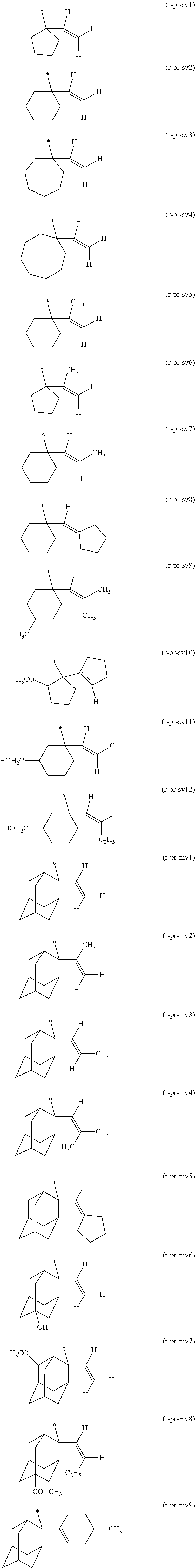

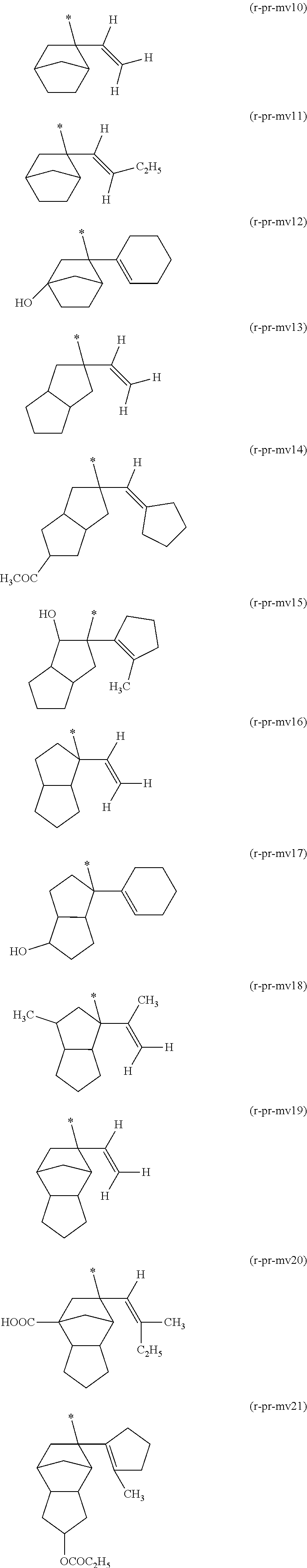

Specific examples of the group represented by Formula (a1-r2-3) are shown below.

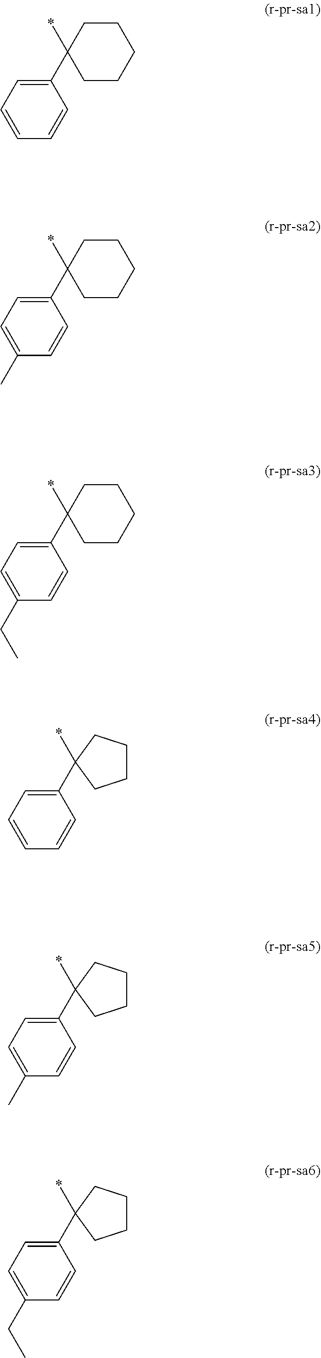 [SMILES: *C1(c2ccccc2)CCCCC1 ; *C1(c2ccc(C)cc2)CCCCC1 ; *C1(c2ccc(CC)cc2)CCCCC1 ; *C1(c2ccccc2)CCCC1 ; *C1(c2ccc(C)cc2)CCCC1 ; *C1(c2ccc(CC)cc2)CCCC1]

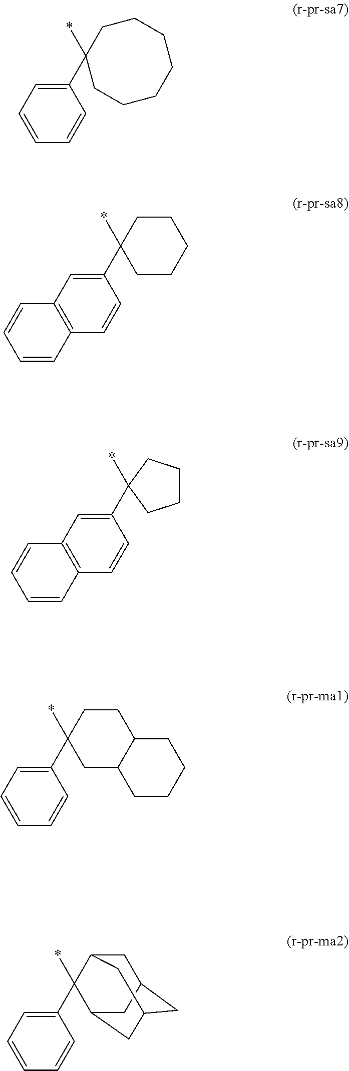 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

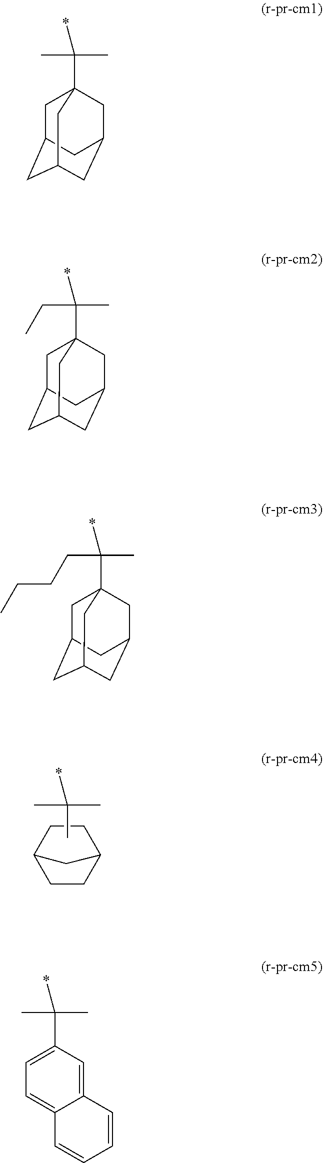

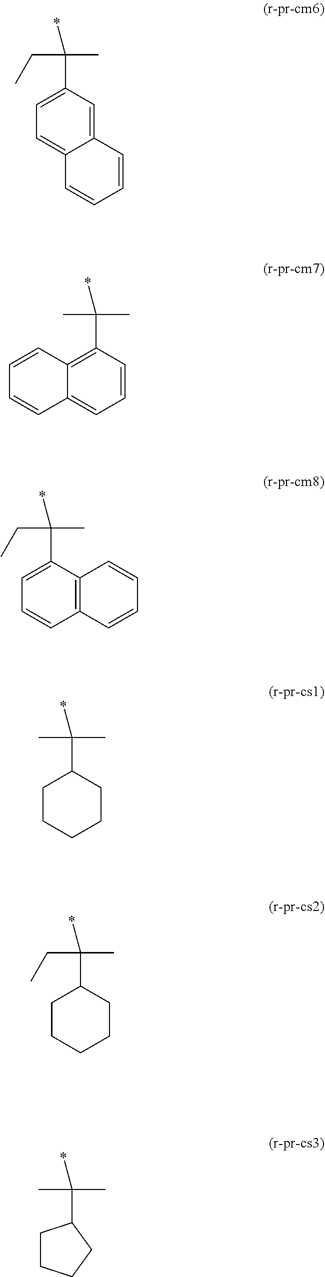

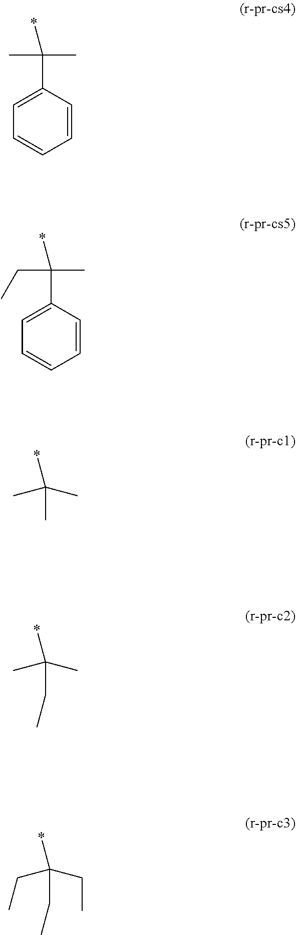

### Tertiary Alkyloxycarbonyl Acid Dissociable Group:

Examples of the acid dissociable group that protects a hydroxyl group among the polar groups include an acid dissociable group (hereinafter, also referred to as “tertiary alkyloxycarbonyl acid dissociable group” for convenience) represented by Formula (a1-r-3).

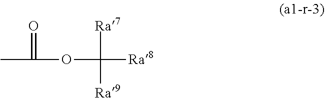

[In the formula, Ra′⁷ to Ra′⁹ each represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by Formula (a1-1) or (a1-2) shown below.

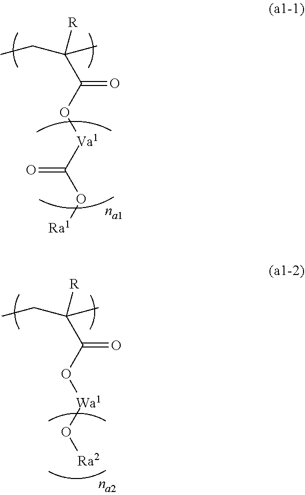

[In the formulae, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ represents an acid dissociable group represented by General Formula (a1-r-1) or (a1-r-2). Wa¹ represents a (nₐ₂+1)-valent hydrocarbon group, nₐ₂ represents an integer of 1 to 3, and Ra² represents an acid dissociable group represented by Formula (a1-r-1) or (a1-r-3)].

In Formula (a1-1), as the alkyl group having 1 to 5 carbon atoms as R, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms and most preferably a hydrogen atom or a methyl group from the viewpoint of the industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH2)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH2CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples thereof include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents an acid dissociable group represented by Formula (a1-r-1) or (a1-r-2).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent to tetravalent and more preferably divalent or trivalent.

In Formula (a1-2), Ra² represents an acid dissociable group represented by Formula (a1-r-1) or (a1-r-3).

Specific examples of the constitutional unit represented by Formula (a1-1) are shown below. In the formulae shown below, R^{a} represents a hydrogen atom, a methyl group, or a trifluoromethyl group.

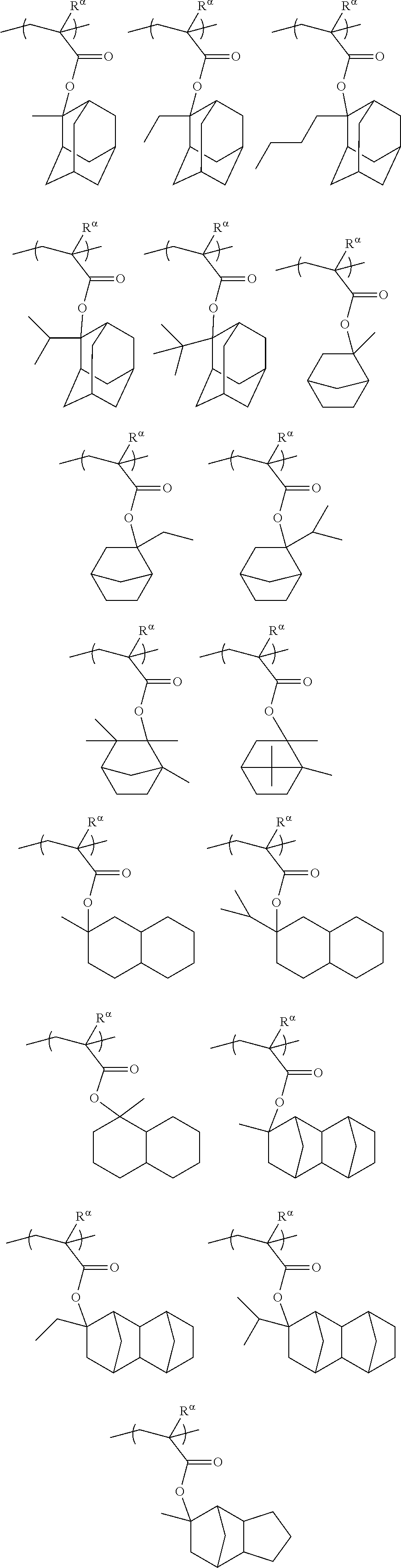

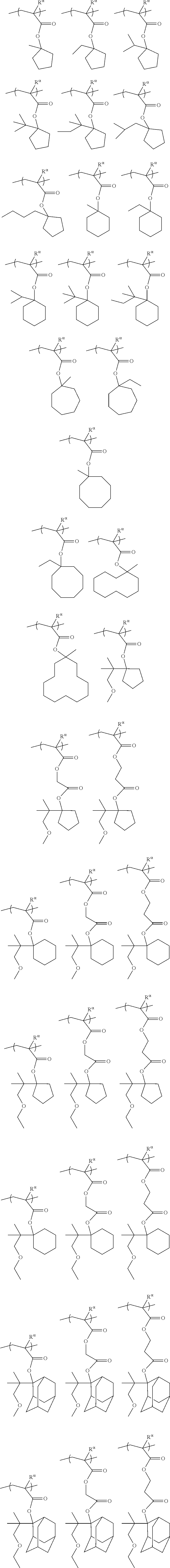

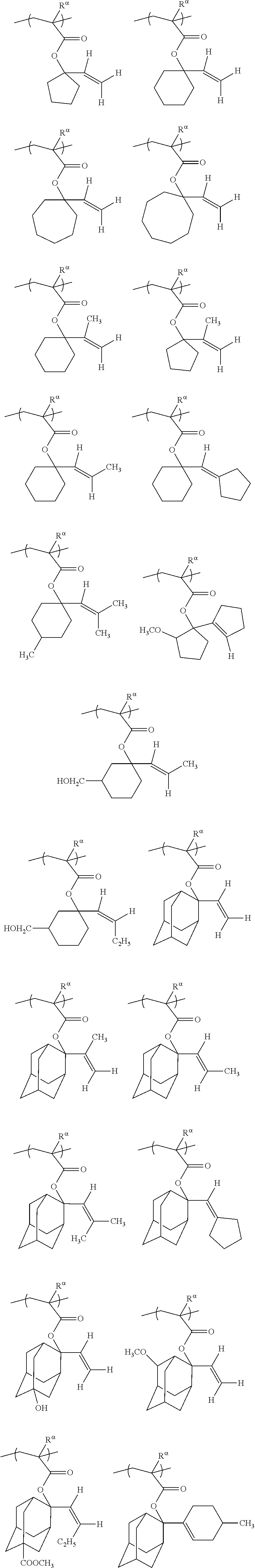

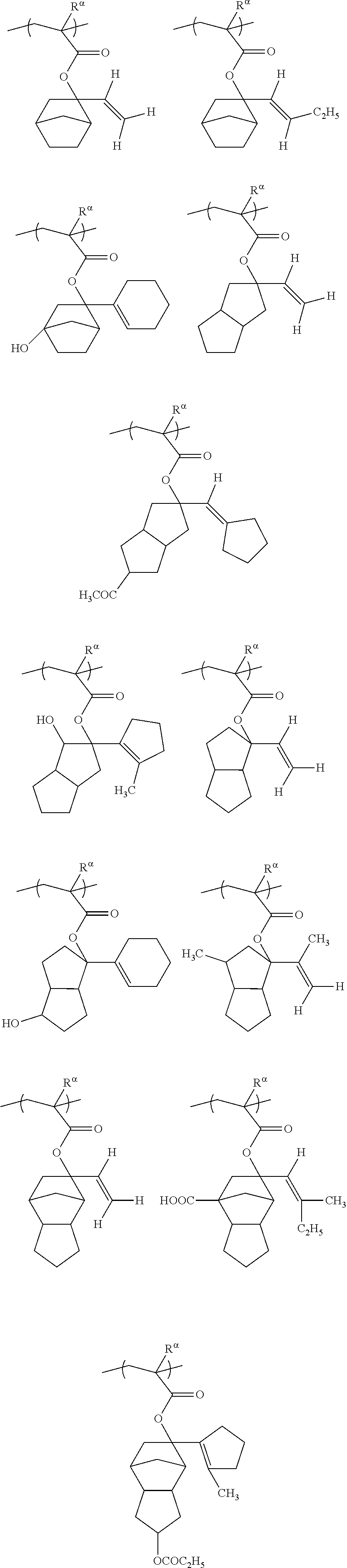

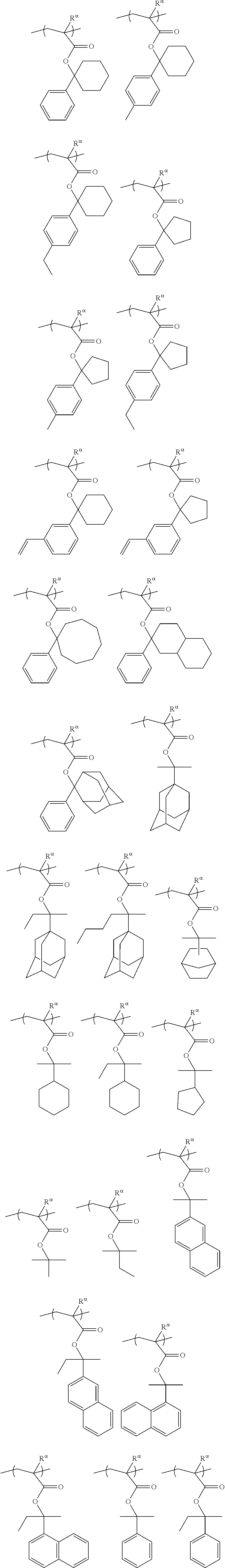

The constitutional unit (a1) included in the component (A1) may be used alone or two or more kinds thereof may be used.

Since the lithography characteristics (the sensitivity, the shape, and the like) are easily improved using electron beams or EUV, a constitutional unit represented by Formula (a1-1) is preferable as the constitutional unit (a1).

Among the examples, as the constitutional unit (a1), those having a constitutional unit represented by Formula (a1-1-1) are particularly preferable.

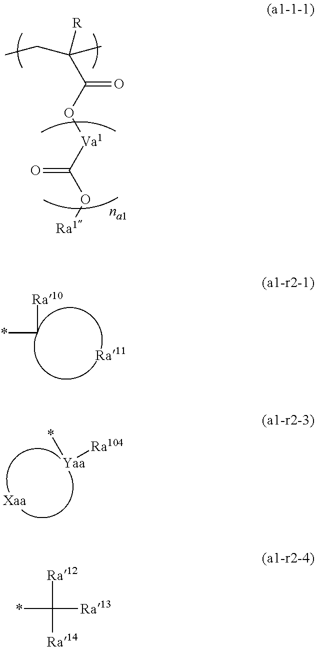

[In the formulae, Ra¹″ represents an acid dissociable group represented by Formula (a1-r2-1), (a1-r2-3), or (a1-r2-4).]

In Formula (a1-1-1), R, Va¹, and nₐ1 each have the same definition as that for R, Va¹, and nₐ1 in Formula (a1-1).

The description of the acid dissociable group represented by Formula (a1-r2-1), (a1-r2-3), or (a1-r2-4) is the same as described above. Among these, it is preferable to select those in which the acid dissociable group is a cyclic group because the reactivity is enhanced for EB or EUV, which is preferable.

In Formula (a1-1-1), Ra¹″ represents an acid dissociable group represented by General Formula (a1-r2-1) among the examples described above.

The proportion of the constitutional unit (a1) in the component (A1) is preferably in a range of 5% to 80% by mole, more preferably in a range of 10% to 75% by mole, still more preferably in a range of 30% to 70% by mole, and particularly preferably in a range of 40% to 70% by mole with respect to the total amount (100% by mole) of all constitutional units constituting the component (A1).

By setting the proportion of the constitutional unit (a1) to be greater than or equal to the lower limits of the above-described preferable ranges, lithography characteristics of enhancement of the sensitivity and the resolution and reduction of the roughness are improved. Further, in a case where the proportion of the constitutional unit (a1) is less than or equal to the upper limits of the above-described preferable ranges, the constitutional unit (a1) and other constitutional units can be balanced, and the lithography characteristics are improved.

### <<Other Constitutional Units>>

Further, the component (A1) may have other constitutional units as necessary in addition to the constitutional unit (a1).

Examples of other constitutional units include a constitutional unit (a2) that contains a lactone-containing cyclic group, a —SO₂-containing cyclic group, or a carbonate-containing cyclic group; a constitutional unit (a3) that contains a polar group-containing aliphatic hydrocarbon group; a constitutional unit (a4) that contains an acid non-dissociable aliphatic cyclic group; a constitutional unit (st) derived from styrene or a styrene derivative; and a constitutional unit derived from a hydroxystyrene or a hydroxystyrene derivative.

### In Regard to Constitutional Unit (a2):

The component (A1) may further have a constitutional unit (a2) (here, those corresponding to the constitutional unit (a1) are excluded) containing a lactone-containing cyclic group, a —SO₂-containing cyclic group, or a carbonate-containing cyclic group, in addition to the constitutional unit (a1).

In a case where the component (A1) is used for forming a resist film, the lactone-containing cyclic group, the —SO₂-containing cyclic group, or the carbonate-containing cyclic group in the constitutional unit (a2) is effective for improving the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group. The lactone-containing cyclic group in the constitutional unit (a2) is not particularly limited, and an optional constitutional unit can be used. Specific examples thereof include groups each represented by Formulae (a2-r-1) to (a2-r-7).

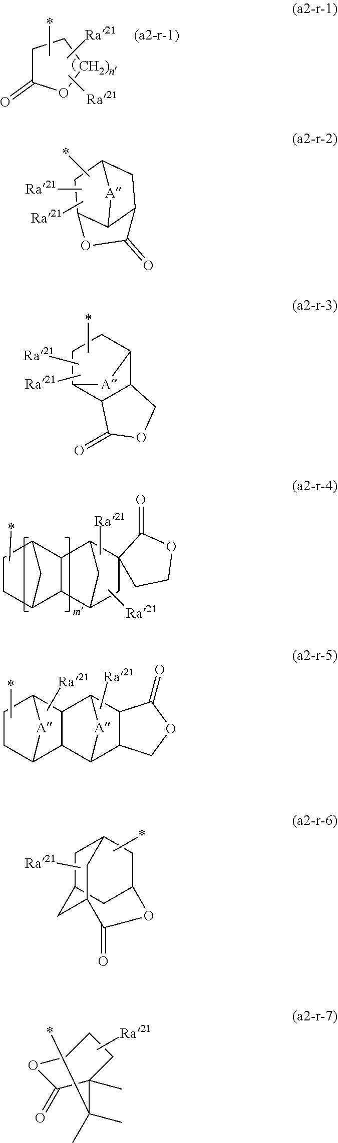

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, a carbonate-containing cyclic group, or a —SO₂-containing cyclic group; A″ represents an alkylene group having 1 to 5 carbon atoms which may have an oxygen atom (—O—) or a sulfur atom (—S—), an oxygen atom, or a sulfur atom; n′ represents an integer of 0 to 2; and m′ represents 0 or 1.]

In General Formulae (a2-r-1) to (a2-r-7), it is preferable that the alkyl group as Ra′²¹ is an alkyl group having 1 to 6 carbon atom. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include a group obtained by substituting some or all hydrogen atoms in the above-described alkyl group as Ra′²¹ with the above-described halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, a lactone-containing cyclic group, a carbonate-containing cyclic group, or a —SO₂-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclodecane, or tetracyclododecane.

Examples of the lactone-containing cyclic group as R″ include the same groups as those for the groups each represented by General Formulae (a2-r-1) to (a2-r-7).

The carbonate-containing cyclic group as R″ has the same definition as that for the carbonate-containing cyclic group described below. Specific examples of the carbonate-containing cyclic group include groups each represented by General Formulae (ax3-r-1) to (ax3-r-3).

The —SO₂-containing cyclic group as R″ is the same as —SO₂-containing cyclic group described below. Specific examples thereof include a group each represented by General Formulae (a5-r-1) to (a5-r-4).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

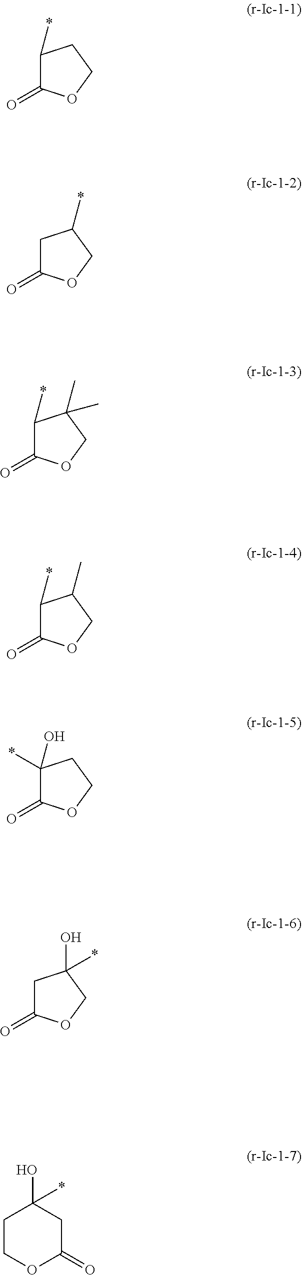

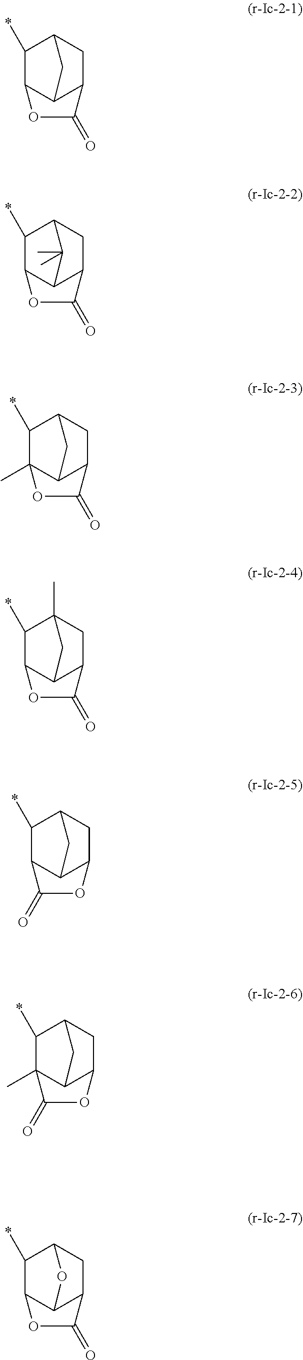

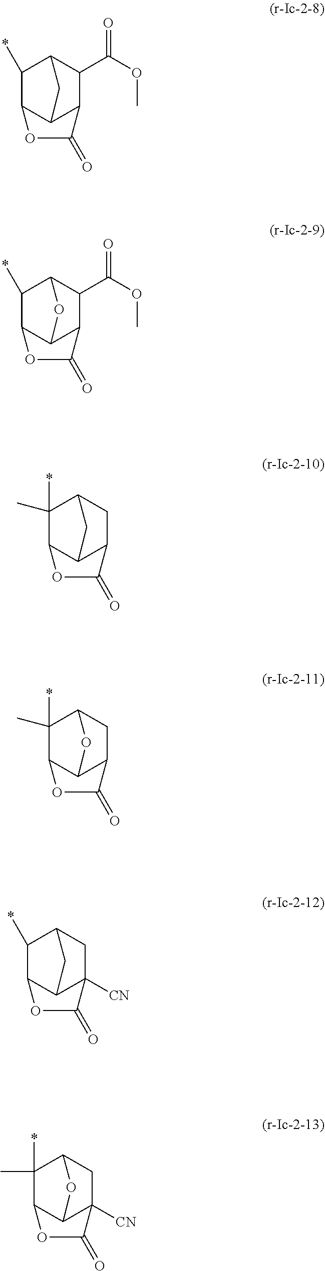

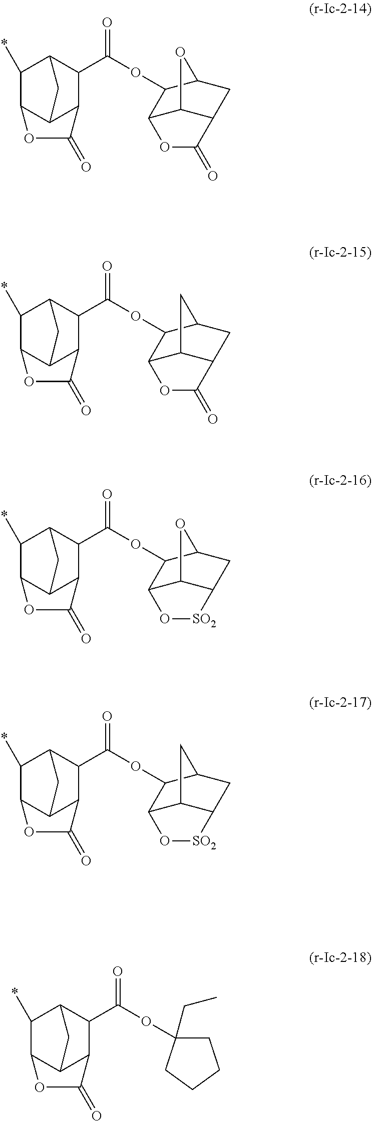

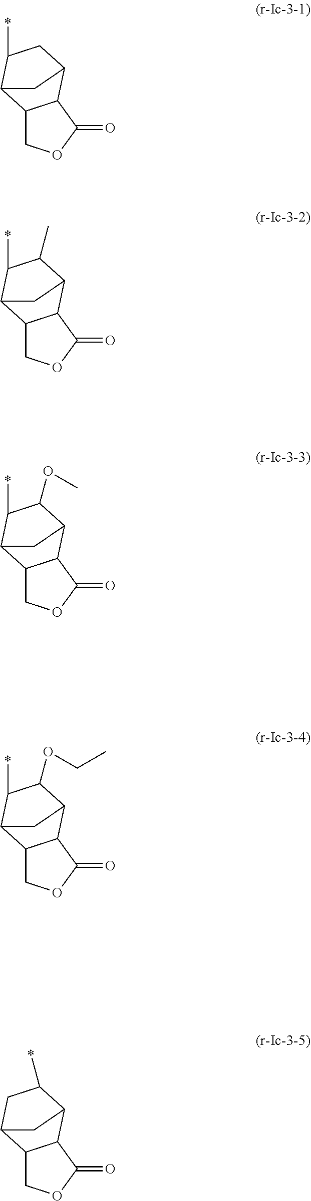

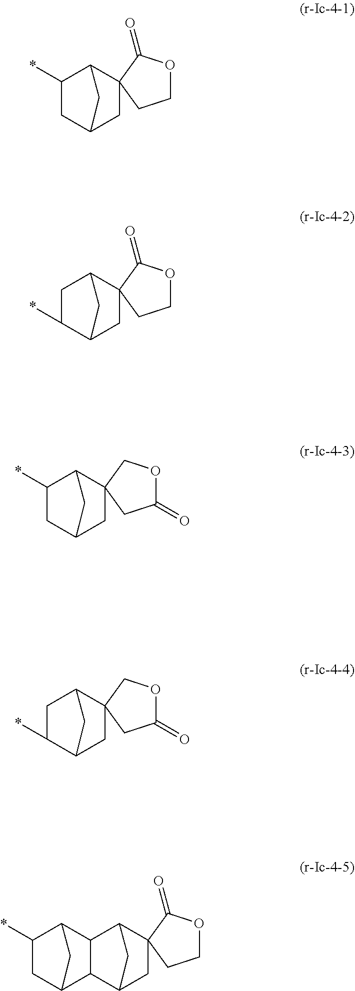

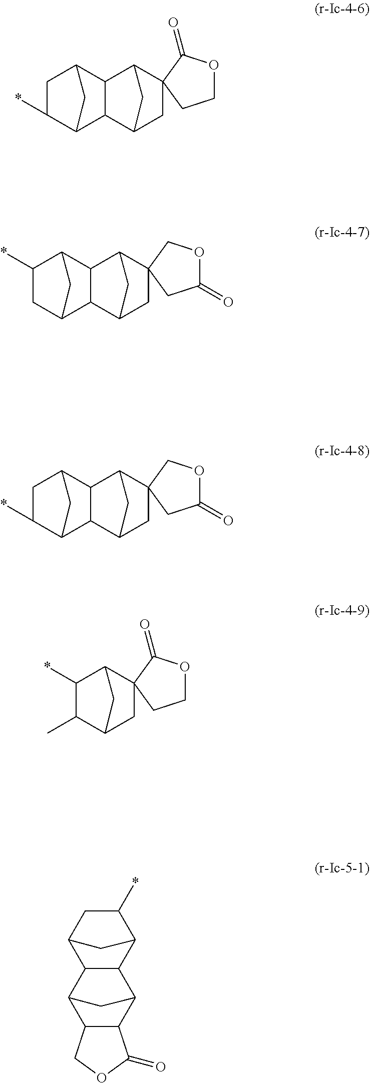

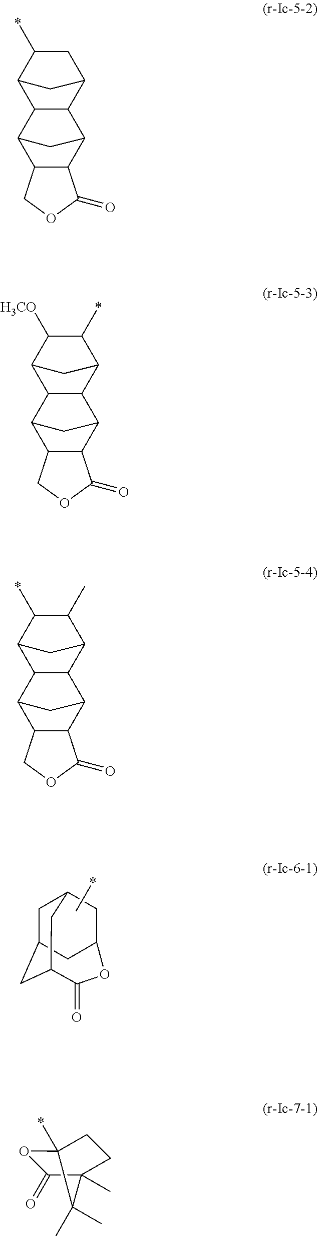

The term “—SO₂-containing cyclic group” denotes a cyclic group having a ring containing —SO₂— in the ring skeleton thereof. Specifically, the —SO₂-containing cyclic group is a cyclic group in which the sulfur atom (S) in —SO₂— forms a part of the ring skeleton of the cyclic group. In a case where the ring containing —SO₂— in the ring skeleton thereof is counted as the first ring and the group has only the ring, the group is referred to as a monocyclic group. In a case where the group further has other ring structures, the group is referred to as a polycyclic group regardless of the structures. The —SO₂-containing cyclic group may be a monocyclic group or a polycyclic group.

The —SO₂-containing cyclic group is particularly preferably a cyclic group containing —O—SO₂— in the ring skeleton thereof, that is, a cyclic group containing a sultone ring in which —O—S— in —O—SO₂— forms a part of the ring skeleton thereof.

More specific examples of the —SO₂-containing cyclic group include groups each represented by General Formulae (a5-r-1) to (a5-r-4).

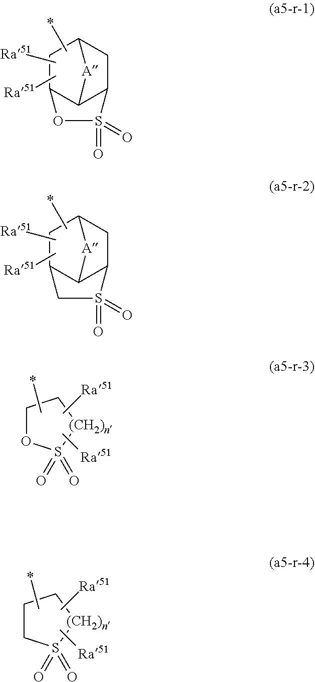

[In the formulae, each Ra′⁵¹ independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, a carbonate-containing cyclic group, or a —SO₂-containing cyclic group, A″ represents an alkylene group having 1 to 5 carbon atoms which may contain an oxygen atom or a sulfur atom, an oxygen atom, or a sulfur atom, and n′ represents an integer of 0 to 2.]

In Formulae (a5-r-1) and (a5-r-2), A″ has the same definition as that for A″ in Formulae (a2-r-2), (a2-r-3) and (a2-r-5).

Examples of each of the alkyl group, the alkoxy group, the halogen atom, the halogenated alkyl group, —COOR″, —OC(═O)R″, and the hydroxyalkyl group as Ra′⁵¹ include the same groups as those for Ra′²¹ in General Formulae (a2-r-1) to (a2-r-7). Specific examples of the groups each represented by Formulae (a5-r-1) to (a5-r-4) are shown below. In the formulae shown below, “Ac” represents an acetyl group.

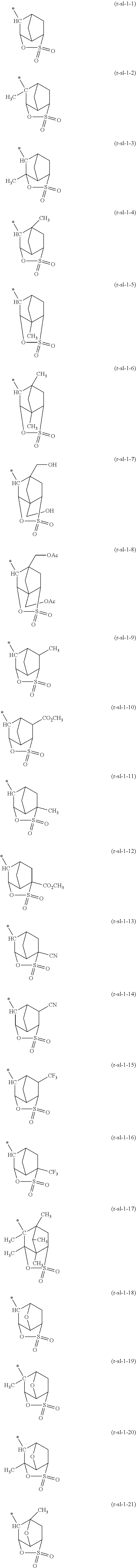

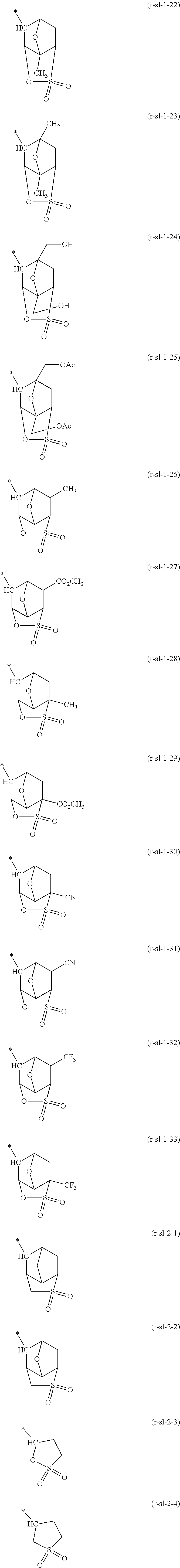

The term “carbonate-containing cyclic group” indicates a cyclic group that has a ring (a carbonate ring) containing —O—C(═O)—O— in the ring skeleton thereof. In a case where the carbonate ring is counted as the first ring and the group has only the carbonate ring, the group is referred to as a monocyclic group. Further, in a case where the group has other ring structures, the group is referred to as a polycyclic group regardless of the structures. The carbonate-containing cyclic group may be a monocyclic group or a polycyclic group.

The carbonate ring-containing cyclic group is not particularly limited, and an optional group can be used. Specific examples thereof include groups each represented by Formulae (ax3-r-1) to (ax3-r-3) shown below.

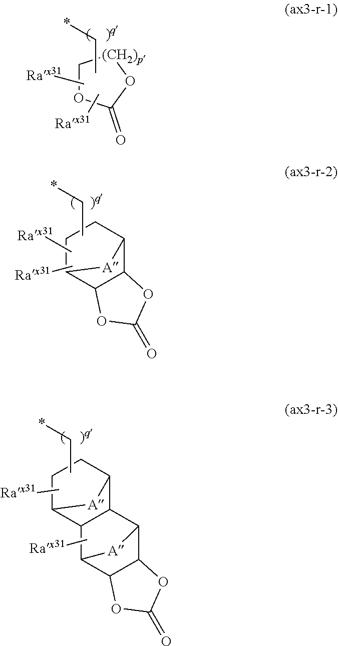

[In the formulae, each Ra′^{x31} independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom. an alkyl group. a lactone-containing cyclic group. a carbonate-containing cyclic group, or a —SO₂-containing cyclic group, A″ represents an alkylene group having 1 to 5 carbon atoms which may contain an oxygen atom or a sulfur atom, an oxygen atom, or a sulfur atom, p′ represents an integer of 0 to 3, and q′ represents 0 or 1.]

In Formulae (ax3-r-2) and (ax3-r-3), A″ has the same definition as that for A″ in Formulae (a2-r-2), (a2-r-3) and (a2-r-5).

Examples of each of the alkyl group, the alkoxy group, the halogen atom, the halogenated alkyl group, —COOR″, —OC(═O)R″, and the hydroxyalkyl group as Ra′³¹ include the same groups as those for Ra′²¹ in General Formulae (a2-r-1) to (a2-r-7).

Specific examples of the groups each represented by Formulae (ax3-r-1) to (ax3-r-3) are shown below.

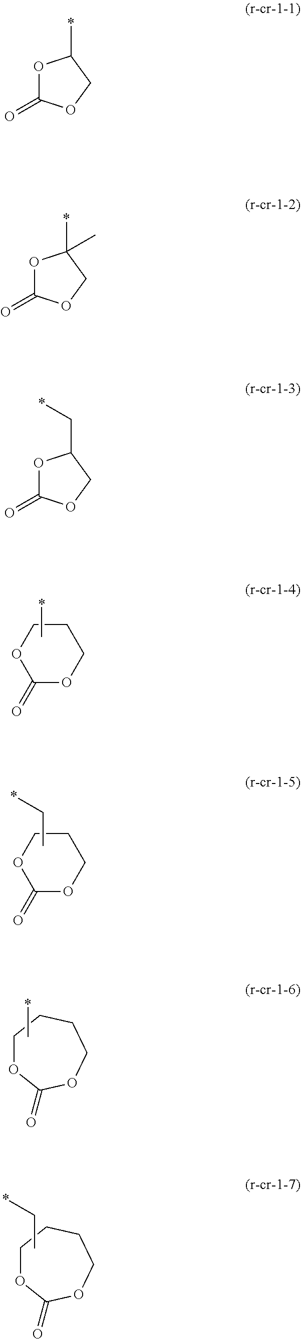

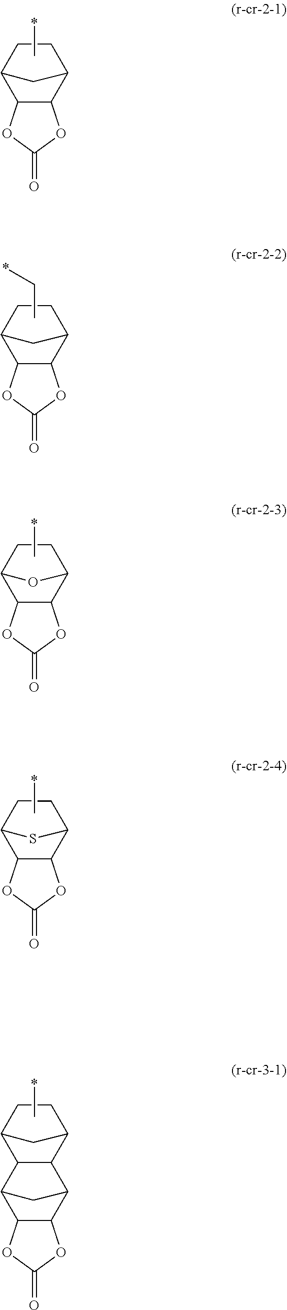

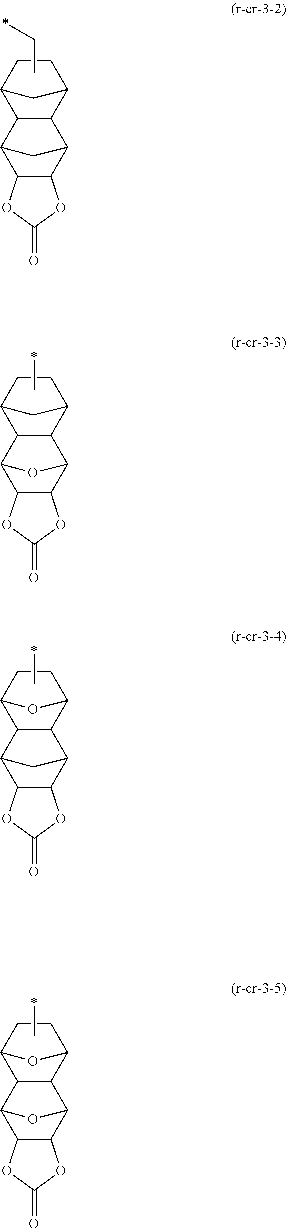

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

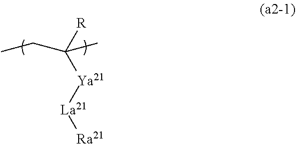

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represents —CO—. Ra²¹ represents a lactone-containing cyclic group, a carbonate-containing cyclic group, or a —SO₂-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having hetero atoms.

Divalent Hydrocarbon Group which May have Substituent:
in a case where Ya²¹ represents a divalent hydrocarbon group which may have a substituent, the hydrocarbon group may be an aliphatic hydrocarbon group or an aromatic hydrocarbon group.

### Aliphatic Hydrocarbon Group as Ya²¹

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

Aliphatic Hydrocarbon Group Having Ring in Structure Thereof Examples of the aliphatic hydrocarbon group having a ring in the structure thereof include a cyclic aliphatic hydrocarbon group which may have a substituent having a hetero atom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in a linear or branched aliphatic hydrocarbon group. Examples of the linear or branched aliphatic hydrocarbon group include the same groups as those described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. As the polycycloalkane, a group having 7 to 12 carbon atoms is preferable. Specific examples of the polycycloalkane include adamantane, norbornane, isobornane, tricyclodecane, and tetracyclododecane.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a hetero atom. As the substituent having a hetero atom, —O—, —C(═O)—O—, —S—, —S(═O)₂—, or —S(═O)₂—O— is preferable.

### Aromatic Hydrocarbon Group as Ya²¹

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with hetero atoms. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (such as biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the same groups as the above-described substituent groups that substitute a hydrogen atom in the cyclic aliphatic hydrocarbon group are exemplary examples.

### Divalent Linking Group Having Hetero Atom:

In a case where Ya²¹ represents a divalent linking group having a hetero atom, preferred examples of the linking group include —O—, —C(═O)—O—, —O—C(═O)—, —C(═O)—, —O—C(═O)—O—, —C(═O)—NH—, —NH—, —NH—C(═NH)— (H may be substituted with a substituent such as an alkyl group or an acyl group), —S—, —S(═O)₂—, —S(═O)₂—O—, and a group represented by General Formula: —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²—S(═O)₂—O—Y²²— [in the formulae, Y²¹ and Y²² each independently represent a divalent hydrocarbon group which may have a substituent, 0 represents an oxygen atom, and m″ represents an integer of 0 to 3]. In a case where the divalent linking group containing a hetero atom is —C(═O)—NH—, —C(═O)—NH—C(═O)—, —NH—, or —NH—C(═NH)—, H may be substituted with a substituent such as an alkyl group, an acyl group, or the like. The substituent (an alkyl group, an acyl group, or the like) has preferably 1 to 10 carbon atoms, more preferably 1 to 8 carbon atoms, and particularly preferably 1 to 5 carbon atoms.

In Formula —Y²¹—O—Y²²—, —Y²¹—O—, —Y²¹—C(═O)—O—, —C(═O)—O—Y²¹—, —[Y²—C(═O)—O]n-Y²²—, —Y²¹—O—C(═O)—Y²²—, or —Y²¹—S(═O)₂—O—Y²²—, Y²¹ and Y²² each independently represent a divalent hydrocarbon group which may have a substituent. Examples of the divalent hydrocarbon group include the same groups as those for the above-described divalent linking group (the divalent hydrocarbon groups which may have a substituent) as Ya²¹.

Y²¹ represents preferably a linear aliphatic hydrocarbon group, more preferably a linear alkylene group, still more preferably a linear alkylene group having 1 to 5 carbon atoms, and particularly preferably a methylene group or an ethylene group.

Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]_{m″}—, —Y²². Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Among the examples, it is preferable that Ya²¹ represents a single bond, an ester bond [—C(═O)—O—], an ether bond (—O—), a linear or branched alkylene group, or a combination thereof.

In Formula (a2-1), Ra²¹ represents a lactone-containing cyclic group, a —SO₂-containing cyclic group, or a carbonate-containing cyclic group.

Suitable examples of the lactone-containing cyclic group, the —SO₂-containing cyclic group, and the carbonate-containing cyclic group as Ra²¹ include groups each represented by General Formulae (a2-r-1) to (a2-r-7), groups each represented by General Formulae (a5-r-1) to (a5-r-4), and groups each represented by General Formulae (ax3-r-1) to (ax3-r-3).

Among the examples, the lactone-containing cyclic group or the —SO₂-containing cyclic group is preferable, a group represented by any of General Formulae (a2-r-1), (a2-r-2), (a2-r-6), and (a5-r-1) is more preferable, and a group represented by any of General Formulae (a2-r-2) and (a5-r-1) is still more preferable. Specifically, a group represented by any of Chemical Formulae (r-1c-1-1) to (r-1c-1-7), (r-1c-2-1) to (r-1c-2-18), (r-1c-6-1), (r-s1-1-1), and (r-s1-1-18) is preferable, a group represented by any of Chemical Formulae (r-1c-2-1) to (r-1c-2-18) and (r-s1-1-1) is more preferable, and a group represented by any of Chemical Formulae (r-1c-2-1), (r-1c-2-12), and (r-s1-1-1) is still more preferable.

The constitutional unit (a2) included in the component (A1) may be used alone or two or more kinds thereof may be used.

In a case where the component (A1) has the constitutional unit (a2), the proportion of the constitutional unit (a2) is preferably in a range of 5% to 60% by mole, more preferably in a range of 5% to 55% by mole, still more preferably in a range of 5% to 50% by mole, and particularly preferably in a range of 5% to 45% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (Ala) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### In Regard to Constitutional Unit (a3):

The component (A1) may further have a constitutional unit (a3) containing a polar group-containing aliphatic hydrocarbon group (here, those corresponding to the constitutional unit (a1) or the constitutional unit (a2) are excluded) in addition to the constitutional unit (a1). In a case where the component (A1) has the constitutional unit (a3), the hydrophilicity of the component (A) is increased, which contributes to improvement of the resolution. Further, the acid diffusion length can be appropriately adjusted.

Examples of the polar group include a hydroxyl group, a cyano group, a carboxy group, or a hydroxyalkyl group in which some hydrogen atoms in the alkyl group have been substituted with fluorine atoms. Among these, a hydroxyl group is particularly preferable.

Examples of the aliphatic hydrocarbon group include a linear or branched hydrocarbon group (preferably an alkylene group) having 1 to 10 carbon atoms and a cyclic aliphatic hydrocarbon group (cyclic group). The cyclic group may be a monocyclic group or a polycyclic group. For example, the cyclic group can be appropriately selected from the plurality of groups that have been proposed in the resins for resist compositions for ArF excimer lasers.

In a case where the cyclic group is a monocyclic group, the number of carbon atoms is more preferably in a range of 3 to 10. Among the examples, constitutional units derived from acrylic acid ester that include an aliphatic monocyclic group containing a hydroxyl group, a cyano group, a carboxy group, or a hydroxyalkyl group in which some hydrogen atoms in the alkyl group have been substituted with fluorine atoms are more preferable. Examples of the monocyclic group include groups obtained by removing two or more hydrogen atoms from a monocycloalkane. Specific examples include groups obtained by removing two or more hydrogen atoms from monocycloalkanes such as cyclopentane, cyclohexane, and cyclooctane. Among these monocyclic groups, a group obtained by removing two or more hydrogen atoms from cyclopentane and a group obtained by removing two or more hydrogen atoms from cyclohexane are industrially preferable.

In a case where the cyclic group is a polycyclic group, the number of carbon atoms in the polycyclic group is more preferably in a range of 7 to 30. Among the examples, constitutional units derived from acrylic acid ester that include an aliphatic polycyclic group containing a hydroxyl group, a cyano group, a carboxy group, or a hydroxyalkyl group in which some hydrogen atoms in the alkyl group have been substituted with fluorine atoms are more preferable. Examples of the polycyclic group include groups in which two or more hydrogen atoms have been removed from bicycloalkane, tricycloalkane, tetracycloalkane, or the like. Specific examples thereof include groups in which two or more hydrogen atoms have been removed from a polycycloalkane such as adamantane, norbornane, isobornane, tricyclodecane, or tetracyclododecane. Among these polycyclic groups, a group in which two or more hydrogen atoms have been removed from adamantane, a group in which two or more hydrogen atoms have been removed from norbornane, or a group in which two or more hydrogen atoms have been removed from tetracyclododecane is industrially preferable.

The constitutional unit (a3) is not particularly limited as long as the constitutional unit contains a polar group-containing aliphatic hydrocarbon group, and an optional constitutional unit can be used.

As the constitutional unit (a3), a constitutional unit derived from acrylic acid ester in which the hydrogen atom bonded to the carbon atom at the α-position may be substituted with a substituent, which is a constitutional unit containing a polar group-containing aliphatic hydrocarbon group is preferable.

In a case where the hydrocarbon group in the polar group-containing aliphatic hydrocarbon group is a linear or branched hydrocarbon group having 1 to 10 carbon atoms, a constitutional unit derived from hydroxyethyl ester of acrylic acid is preferable as the constitutional unit (a3).

Further, in a case where the hydrocarbon group in the polar group-containing aliphatic hydrocarbon group is a polycyclic group, a constitutional unit represented by Formula (a3-1), a constitutional unit represented by Formula (a3-2), or a constitutional unit represented by Formula (a3-3) is preferable as the constitutional unit (a3). Further, in a case where the hydrocarbon group in the polar group-containing aliphatic hydrocarbon group is a monocyclic group, a constitutional unit represented by Formula (a3-4) is preferable as the constitutional unit (a3).

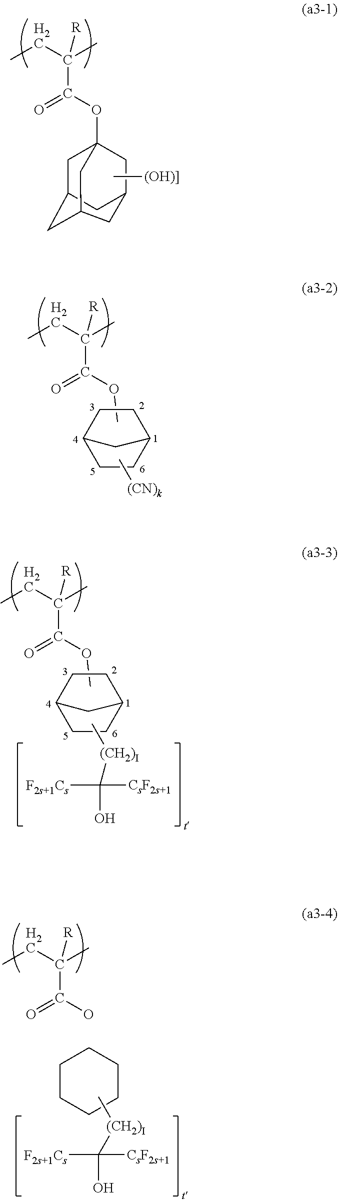

[In the formulae, R has the same definition as described above, j represents an integer of 1 to 3, k represents an integer of 1 to 3, t′ represents an integer of 1 to 3, 1 represents an integer of 0 to 5, and s represents an integer of 1 to 3.]

In Formula (a3-1), j represents preferably 1 or 2 and more preferably 1. In a case where j represents 2, it is preferable that the hydroxyl groups is bonded to the 3- and 5-positions of the adamantyl group. In a case where j represents 1, it is preferable that the hydroxyl group is bonded to the 3-position of the adamantyl group.

It is preferable that j represents 1, and it is particularly preferable that the hydroxyl group is bonded to the 3-position of the adamantyl group.

In Formula (a3-2), it is preferable that k represents 1. It is preferable that the cyano group is bonded to the 5-th or 6-th position of the norbornyl group.

In Formula (a3-3), it is preferable that t′ represents 1. It is preferable that 1 represents 1. It is preferable that s represents 1. Further, it is preferable that a 2-norbornyl group or 3-norbornyl group is bonded to the terminal of the carboxy group of the acrylic acid. It is preferable that the fluorinated alkyl alcohol is bonded to the 5- or 6-position of the norbornyl group.

In Formula (a3-4), it is preferable that t′ represents 1 or 2. It is preferable that 1 represents 0 or 1. It is preferable that s represents 1. It is preferable that the fluorinated alkyl alcohol is bonded to the 3- or 5-position of the cyclohexyl group.

The constitutional unit (a3) included in the component (A1) may be used alone or two or more kinds thereof may be used.

In a case where the component (A1) has the constitutional unit (a3), the proportion of the constitutional unit (a3) is preferably in a range of 1% to 30% by mole, more preferably in a range of 2% to 25% by mole, and still more preferably in a range of 5% to 25% by mole, with respect to the total amount (100% by mole) of all constitutional units constituting the component (A1).

In a case where the proportion of the constitutional unit (a3) is set to be greater than or equal to the lower limits of the above-described preferable ranges, the effect to be obtained by allowing the component (Ala) to have the constitutional unit (a3) is sufficiently obtained by the above-described effects. Further, in a case where the proportion thereof is set to be less than or equal to the upper limits of the above-described preferable ranges, the constitutional unit (a3) and other constitutional units can be balanced, and the lithography characteristics are improved.

### In Regard to Constitutional Unit (a4):

The component (A1) may further have a constitutional unit (a4) containing an acid non-dissociable aliphatic cyclic group in addition to the constitutional unit (a1).

In a case where the component (A1) has the constitutional unit (a4), the dry etching resistance of a resist pattern to be formed is improved. Further, the hydrophobicity of the component (A) is increased. The improvement of the hydrophobicity contributes to improvement of the resolution, the resist pattern shape, and the like particularly in a case of the solvent developing process.

The term “acid non-dissociable cyclic group” in the constitutional unit (a4) denotes a cyclic group remaining in the constitutional unit without being dissociated due to the action of an acid in a case of generation of an acid in the resist composition upon light exposure (for example, an acid is generated from the component (B) or a constitutional unit that generates an acid upon light exposure).

As the constitutional unit (a4), for example, a constitutional unit derived from acrylic acid ester containing an acid non-dissociable aliphatic cyclic group or the like is preferable. As the cyclic group, a plurality of cyclic groups which have been known in the related art as those used for resin components of resist compositions for an ArF excimer laser, a KrF excimer laser (preferably an ArF excimer laser), and the like can be used.

It is particularly preferable that the cyclic group is at least one selected from a tricyclodecyl group, an adamantyl group, a tetracyclododecyl group, an isobornyl group, and a norbornyl group from the viewpoint of the industrial availability or the like. These polycyclic groups may have a linear or branched alkyl group having 1 to 5 carbon atoms as a substituent.

Specific examples of the constitutional unit (a4) include constitutional units each represented by Formulae (a4-1) to (a4-7).

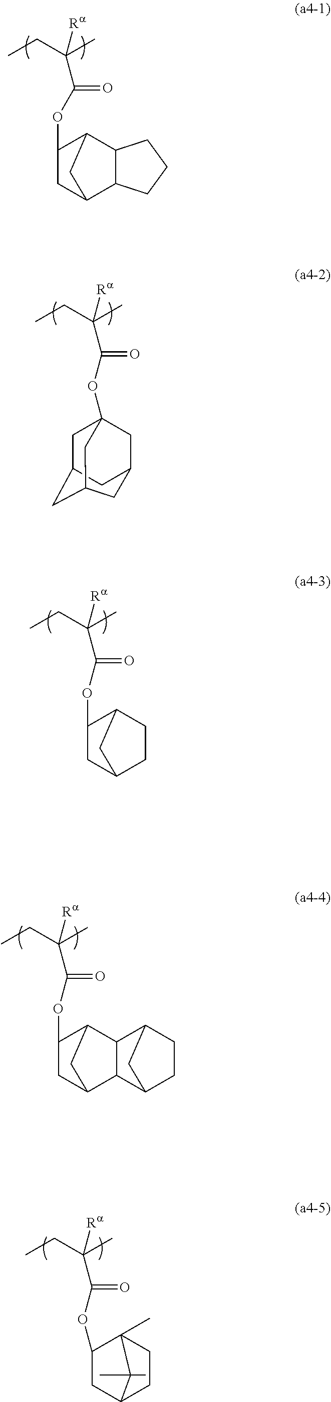

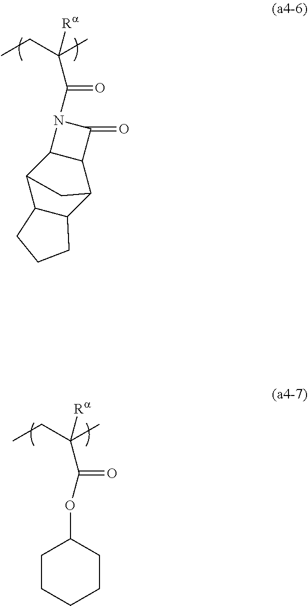

[In the formulae, R^{a} has the same definition as described above.]

The constitutional unit (A4) included in the component (A1) may be used alone or two or more kinds thereof may be used.

In a case where the component (A1) contains the constitutional unit (A4), the proportion of the constitutional unit (A4) is preferably in a range of 1% to 40% by mole and more preferably in a range of 5% to 20% by mole with respect to the total amount (100% by mole) of all constitutional units constituting the component (A1).

In a case where the proportion of the constitutional unit (a4) is set to be greater than or equal to the lower limits of the above-described preferable ranges, the effect to be obtained by allowing the component (A1a) to have the constitutional unit (a4) is sufficiently obtained by the above-described effects. Further, in a case where the proportion thereof is set to be less than or equal to the upper limits of the above-described preferable ranges, the constitutional unit (a4) and other constitutional units can be balanced.

In Regard to Constitutional Unit (a10):

The constitutional unit (a10) is a constitutional unit represented by General Formula (a10-1).

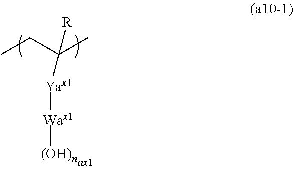

[In the formulae, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In Formula (a10-1), R represents a hydrogen atom, an alkyl group having 1 to 5 carbon atoms, or a halogenated alkyl group having 1 to 5 carbon atoms. R has the same definition as that for R in Formula (a01-1).

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a hetero atom. Examples of the divalent linking group as Ya^{x1} include the same groups as those for the divalent linking group as Ya²¹ in Formula (a2-1).

Among these, Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x}] include a group in which (nₐₓ₁+1) hydrogen atoms have been removed from an aromatic ring which may have a substituent. The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} include a group in which (nₐₓ₁+1) hydrogen atoms have been removed from an aromatic compound (such as biphenyl or fluorene) having an aromatic ring which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group. Examples of the alkyl group, the alkoxy group, the halogen atom, and the halogenated alkyl group as the substituent include those given as examples of the substituent of the cyclic aliphatic hydrocarbon group as Yₐₓ₁. As the substituent, a linear or branched alkyl group having 1 to 5 carbon atoms is preferable, a linear or branched alkyl group having 1 to 3 carbon atoms is more preferable, an ethyl group or a methyl group is still more preferable, and a methyl group is particularly preferable. It is preferable that the aromatic hydrocarbon group as Wₐₓ₁ has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{a} represents a hydrogen atom, a methyl group, or a trifluoromethyl group.

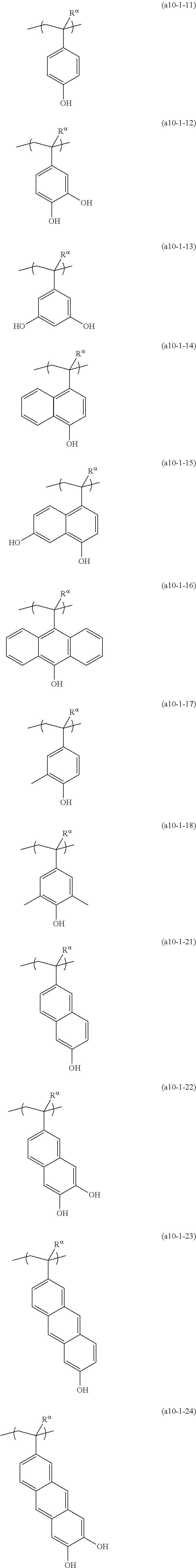

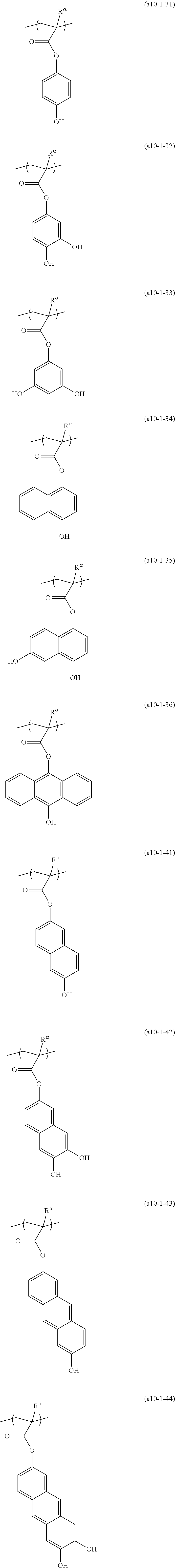

The constitutional unit (a10) included in the component (A1) may be used alone or two or more kinds thereof may be used.

In a case where the component (A1) has the constitutional unit (a10), the proportion of the constitutional unit (a10) in the component (A1) is preferably in a range of 5% to 80% by mole, more preferably in a range of 5% to 70% by mole, and still more preferably in a range of 10% to 60% by mole with respect to the total amount (100% by mole) of all constitutional units constituting the component (A1).

In a case where the proportion of the constitutional unit (a10) is greater than or equal to the lower limits of the above-described preferable ranges, the sensitivity is more likely to be increased. In a case where the proportion of the constitutional unit (a10) is less than or equal to the upper limits of the above-described preferable ranges, the balance between the constitutional unit (a10) and other constitutional units is likely to be achieved.

### In Regard to Constitutional Unit (St):

The constitutional unit (st) is a constitutional unit derived from styrene or a styrene derivative thereof. The phrase “constitutional unit derived from styrene” means a constitutional unit that is formed by the cleavage of an ethylenic double bond of styrene. The expression “constitutional unit derived from a styrene derivative” denotes a constitutional unit formed by cleavage of an ethylenic double bond of a styrene derivative (here, those corresponding to the constitutional unit (a10) are excluded).

The term “styrene derivative” denotes a compound in which at least some hydrogen atoms of styrene are substituted with a substituent. Examples of the styrene derivative include those in which the hydrogen atom at the α-position of styrene is substituted with a substituent, those in which one or more hydrogen atoms in the benzene ring of styrene are substituted with a substituent, and those in which the hydrogen atom at the α-position of styrene and one or more hydrogen atoms of the benzene ring of styrene are substituted with a substituent.

Examples of the substituent that substitutes the hydrogen atom at the α-position of styrene include an alkyl group having 1 to 5 carbon atoms and a halogenated alkyl group having 1 to 5 carbon atoms.

As the alkyl group having 1 to 5 carbon atoms, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

As the substituent that substitutes the hydrogen atom at the α-position of styrene, an alkyl group having 1 to 5 carbon atoms or a fluorinated alkyl group having 1 to 5 carbon atoms is preferable, an alkyl group having 1 to 3 carbon atoms or a fluorinated alkyl group having 1 to 3 carbon atoms is more preferable, and a methyl group is still more preferable from the viewpoint of industrial availability.

Examples of substituents that substitutes the hydrogen atom of the benzene ring of styrene include an alkyl group, an alkoxy group, a halogen atom, and a halogenated alkyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups have been substituted with the above-described halogen atoms.

As the substituent that substitutes the hydrogen atom of the benzene ring of styrene, an alkyl group having 1 to 5 carbon atoms is preferable, a methyl group or an ethyl group is more preferable, and a methyl group is still more preferable.

As the constitutional unit (st), a constitutional unit derived from styrene or a constitutional unit derived from a styrene derivative in which the hydrogen atom at the α-position of styrene is substituted with an alkyl group having 1 to 5 carbon atoms or a halogenated alkyl group having 1 to 5 carbon atoms is preferable, a constitutional unit derived from styrene or a constitutional unit derived from a styrene derivative in which the hydrogen atom at the α-position of styrene is substituted with a methyl group is more preferable, and a constitutional unit derived from styrene is still more preferable.

The constitutional unit (st) included in the component (A1) may be used alone or two or more kinds thereof may be used.

In a case where the component (A1) has the constitutional unit (st), the proportion of the constitutional unit (st) is preferably in a range of 1% to 30% by mole and more preferably in a range of 3% to 20% by mole with respect to the total amount (100% by mole) of all constitutional units constituting the component (A1).

The component (A1) contained in the resist composition may be used alone or a combination of two or more kinds thereof may be used.

In the resist composition according to the present embodiment, examples of the component (A1) include a polymer compound having a repeating structure of the constitutional unit (a1), and preferred examples thereof include a polymer compound having a repeating structure of the constitutional unit (a1) and the constitutional unit (a10).

Among these, suitable examples of the component (A1) include a polymer compound having a repeating structure of the constitutional unit (a1), the constitutional unit (a10), and the constitutional unit (a2); and a polymer compound having a repeating structure of the constitutional unit (a1), the constitutional unit (a10), and the constitutional unit (a3).

Such a component (A1) can be produced by dissolving a monomer, from which each constitutional unit is derived, in a polymerization solvent and adding a radical polymerization initiator such as azobisisobutylonitrile (AIBN) or dimethyl azobisisobutyrate (for example, V-601) to the solution so that the polymerization is carried out.

Alternatively, such a component (A1) can be produced by dissolving a monomer from which the constitutional unit (a1) is derived and a monomer from which constitutional units (for example, the constitutional unit (a2)) other than the constitutional unit (a1) are derived as necessary in a polymerization solvent, adding the above-described radical polymerization initiator to the solution, and performing polymerization.

Further, a —C(CF₃)₂—OH group may be introduced into the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 2000 to 30000, and still more preferably in a range of 3000 to 20000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern is excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### in Regard to Component (A2)

In the resist composition of the present embodiment, a base material component (hereinafter, also referred to as “component (A2)”) which does not correspond to the component (A1) and whose solubility in a developing solution is changed due to the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound is used alone or a combination of two or more kinds thereof may be used.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and improved roughness is likely to be formed.

In the resist composition of the present embodiment, the content of the component (A) may be adjusted according to the thickness of the resist film intended to be formed.

### <Acid Generator Component (B)>

The component (B) in the resist composition according to the present embodiment contains a compound (B0) represented by General Formula (b0) (hereinafter, also referred to as “component (B0)”).

### <<Compound (B0)>>

The component (B0) is a compound represented by General Formula (b0).

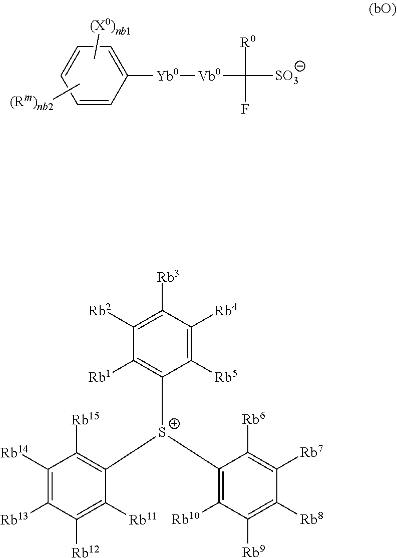

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, Yb⁰ represents a divalent linking group or a single bond, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group.]

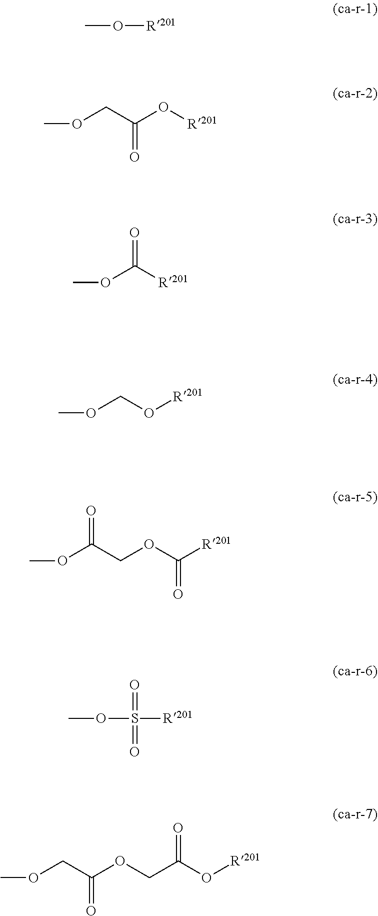

[In the formulae, R′^{201's each independently represent a hydrogen atom, a cyclic group which may have a substituent, a chain-like alkyl group which may have a substituent, or a chain-like alkenyl group which may have a substituent.]}

### {Anion Moiety of Component (B0)}

In General Formula (b0), X⁰ represents a bromine atom or an iodine atom and preferably an iodine atom.

In General Formula (b0), R^{m} represents a hydroxy group, an alkyl group, a fluorine atom, or a chlorine atom. The alkyl group as R^{m} is preferably an alkyl group having 1 to 5 carbon atoms and more preferably a methyl group or an ethyl group.

In General Formula (b0), nb1 represents an integer of 1 to 5, and nb2 represents an integer of 0 to 4, where 1≤nb1+nb2≤5 is satisfied.

nb1 represents preferably an integer of 1 to 3, more preferably 2 or 3, and still more preferably 3.

nb2 represents preferably an integer of 0 to 3, more preferably 0 or 1, and still more preferably 0.

In General Formula (b0), Yb⁰ represents a divalent linking group or a single bond. Suitable examples of the divalent linking group as Yb⁰ include a divalent linking group containing an oxygen atom.

In a case where Yb⁰ represents a divalent linking group containing an oxygen atom, Yb⁰ may contain an atom other than the oxygen atom. Examples of the atom other than the oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination.

In General Formula (b0), Vb⁰ represents an alkylene group, a fluorinated alkylene group, or a single bond.

The alkylene group and the fluorinated alkylene group as Vb⁰ each have preferably 1 to 4 carbon atoms and more preferably 1 to 3 carbon atoms. Examples of the fluorinated alkylene group as Vb⁰ include a group obtained by substituting some or all hydrogen atoms in an alkylene group with a fluorine atom. Among these, Vb⁰ represents preferably an alkylene group having 1 to 4 carbon atoms, a fluorinated alkylene group having 1 to 4 carbon atoms, or a single bond, more preferably a group obtained by substituting some hydrogen atoms of an alkylene group having 1 to 3 carbon atoms with a fluorine atom or a single bond, and still more preferably —CH(CF₃)— or a single bond.

In General Formula (b0), R⁰ represents a hydrogen atom, a fluorinated alkyl group having 1 to 5 carbon atoms, or a fluorine atom. R⁰ represents preferably a fluorine atom or a perfluoroalkyl group having 1 to 5 carbon atoms and more preferably a fluorine atom.

In the present embodiment, the anion moiety of the component (B0) is preferably an anion represented by General Formula (b0-an0).

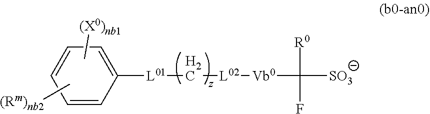

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰¹ and L⁰² each independently represent a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—. R^{a}'s each independently represent a hydrogen atom or an alkyl group. z represents an integer of 0 to 10. Vb⁰ represents a single bond, an alkylene group, or a fluorinated alkylene group, R⁰ represents a hydrogen atom, a fluorinated alkyl group having 1 to 5 carbon atoms, or a fluorine atom.

X⁰, R^{m}, nb1, nb2, Vb⁰, and R⁰ in General Formula (b0-an0) each have the same definition as that for X⁰, R¹, nb1, nb2, Vb⁰, and R⁰ in General Formula (b0).

In General Formula (b0-an0), L⁰¹ and L⁰² each independently represent a single bond, an alkylene group, —O—, —CO—, —OCO—, —COO—, —SO²—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—. R^{a}'s each independently represent a hydrogen atom or an alkyl group.

The alkylene group as L⁰¹ and L⁰² and the alkyl group as R^{a} each have preferably 1 to 4 carbon atoms and more preferably 1 to 3 carbon atoms.

In General Formula (b0-an0), among the examples, it is preferable that at least one of L⁰¹ or L⁰² represents —OCO— or —COO— and more preferable that L⁰¹ represents —OCO— or —COO— and L⁰² represents a single bond, —OCO—, or —COO—.

More specifically, in General Formula (b0-an0), -L⁰¹-(CH₂)z-L⁰²-Vb⁰- is preferably —COO-Vb⁰-, —OCO-Vb⁰-, or —COO—(CH₂)_{z}—COO-Vb⁰-.

In General Formula (b0-an0), z represents an integer of 0 to 10, preferably an integer of 0 to 5, and more preferably an integer of 0 to 3.

Specific examples of the anion moiety of the component (B0) are shown below.

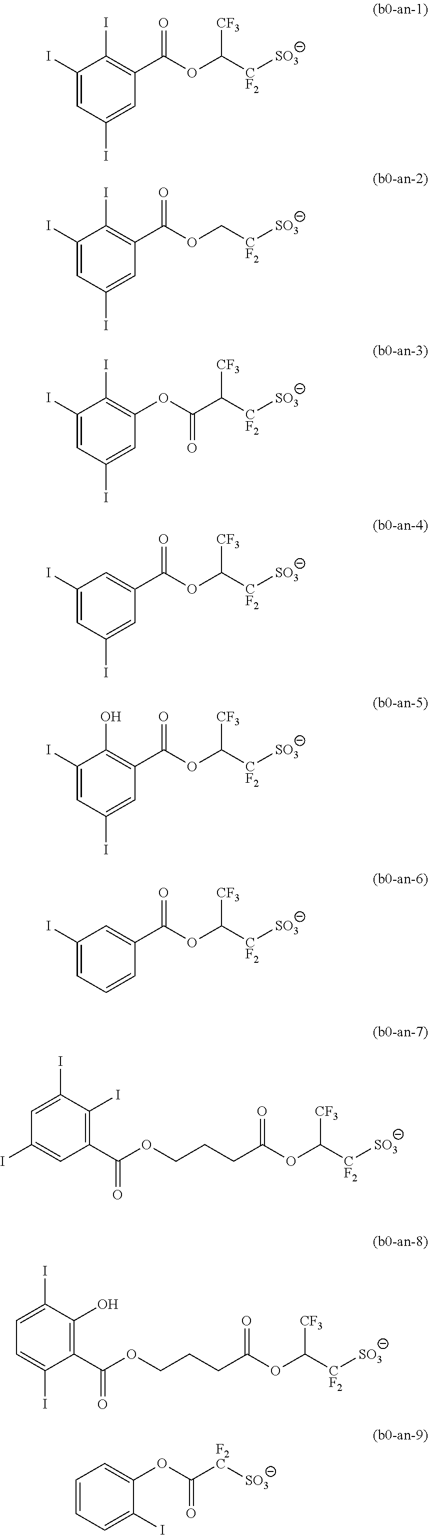

As the anion moiety of the component (B0), an anion represented by any one of Formulae (b0-an-1) to (b0-an-9) is preferable, and an anion represented by any of Formulae (b0-an-1) to (b0-an-7) is more preferable.

### {Cation Moiety of Component (B0)}

In General Formula (b0), examples of the alkyl group as Rb¹ to Rb¹⁵ include a linear or branched alkyl group.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

In General Formula (b0), a fluorine atom is preferable as the halogen atom as Rb¹ to Rb⁵.

In General Formula (b0), the halogenated alkyl group as Rb¹ to Rb¹⁵ is a group in which some or all hydrogen atoms in the alkyl group have been substituted with halogen atoms. Among these, a fluorine atom is preferable as the halogen atom.

In General Formula (b0), the aryl group as Rb¹ to Rb¹⁵ is preferably an aryl group having 3 to 30 carbon atoms, more preferably an aryl group having 5 to 30 carbon atoms, still more preferably an aryl group having 5 to 20 carbon atoms, even still more preferably an aryl group having 6 to 15 carbon atoms, and most preferably an aryl group having 6 to 10 carbon atoms. Here, the number of carbon atoms in a substituent is not included in the number of carbon atoms.

Specific examples of the aromatic ring contained in the aryl group as Rb¹ to Rb¹⁵ include benzene, fluorene, naphthalene, anthracene, phenanthrene, biphenyl, or an aromatic heterocyclic ring in which some carbon atoms constituting these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group in the aryl group as Rb¹ to Rb¹⁵ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In General Formulae (ca-r-1) to (ca-r-7), the cyclic group as R′²⁰¹ is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′^{201 is a hydrocarbon group having an aromatic ring. The aromatic hydrocarbon group has preferably} 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring in the aromatic hydrocarbon group as R′²⁰¹ include benzene, fluorene, naphthalene, anthracene, phenanthrene, biphenyl, and an aromatic heterocyclic ring obtained by substituting some carbon atoms constituting these aromatic rings with a hetero atom. Examples of the hetero atom in the aromatic heterocyclic rings include an oxygen atom, a sulfur atom, and a nitrogen atom. Specific examples of the aromatic hydrocarbon group as R′²⁰¹ include a group (an aryl group such as a phenyl group or a naphthyl group) obtained by removing one hydrogen atom from the above-described aromatic ring and a group (an arylalkyl group such as a benzyl group, a phenethyl group, a 1-naphthylmethyl group, a 2-naphthylmethyl group, a 1-naphthylethyl group, or a 2-naphthylethyl group) obtained by substituting one hydrogen atom in the aromatic ring with an alkylene group. The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include aliphatic hydrocarbon groups containing a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring polycyclic skeleton such as a cyclic group having a steroid skeleton are preferable as the polycycloalkane.

Among these, the cyclic aliphatic hydrocarbon group as R′²⁰¹ is preferably a group obtained by removing one or more hydrogen atoms from a monocycloalkane or a polycycloalkane, more preferably a group obtained by removing one hydrogen atom from a polycycloalkane, particularly preferably an adamantyl group or a norbornyl group, and most preferably an adamantyl group.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R′²⁰¹ may contain a hetero atom such as a heterocyclic ring. Specific examples thereof include lactone-containing cyclic groups each represented by General Formulae (a2-r-1) to (a2-r-7), —SO₂-containing cyclic group each represented by General Formulae (a5-r-1) to (a5-r-4), and other heterocyclic groups each represented by Chemical Formulae (r-hr-1) to (r-hr-16).

Examples of the substituent of the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

In General Formulae (ca-r-1) to (ca-r-7), the chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

In General Formulae (ca-r-1) to (ca-r-7), the chain-like alkenyl group as R′²⁰¹ may be linear or branched and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent in the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, or the chain-like alkenyl group which may have a substituent as R′²⁰¹ include the same groups as those for the acid dissociable group represented by Formula (a1-r-2) as the cyclic group which may have a substituent or the chain-like alkyl group which may have a substituent, in addition to the groups described above.

Among these, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (a5-r-1) to (a5-r-4).

In General Formula (b0), in a case where Rb¹⁰ and Rb¹¹ are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂—, —SO₃—, —COO—, —CONH— or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

Among these, a dibenzothiophene ring is preferable as the ring formed together with the sulfur atom in the formula by Rb¹⁰ and Rb¹¹ being bonded to each other.

In General Formula (b0), at least two of Rb¹ to Rb⁵ represent a fluorine atom, or at least one of Rb¹ to Rb⁵ represents a perfluoroalkyl group.

Examples of the perfluoroalkyl group as Rb¹ to Rb⁵ include a trifluoromethyl group, a pentafluoroethyl group, and a heptafluoropropyl group. Among the examples, a trifluoromethyl group is preferable as the perfluoroalkyl group as Rb¹ to Rb⁵.

From the viewpoint of the stability of the compound (B), the number of fluorine atoms contained in Rb¹ to Rb¹⁵ in General Formula (b0) is preferably in a range of 3 to 6 and more preferably in a range of 4 to 6.

Further, in General Formula (b0), in a case where three to six of Rb¹ to Rb¹⁵ represent a fluorine atom, it is preferable that at least two of Rb¹ to Rb⁵ represent a fluorine atom and at least one of Rb⁶ to Rb¹⁰ represents a fluorine atom.

Specific examples of the cation moiety of the component (B0) are shown below.

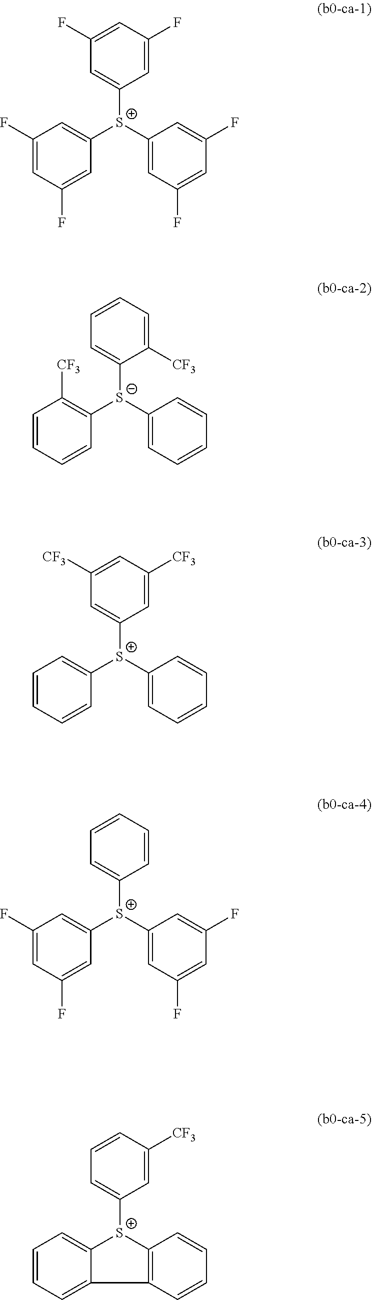 [SMILES: Fc1cc(F)cc([S+](c2cc(F)cc(F)c2)c2cc(F)cc(F)c2)c1 ; FC(F)(F)c1ccccc1[S-](c1ccccc1)c1ccccc1C(F)(F)F ; FC(F)(F)c1cc([S+](c2ccccc2)c2ccccc2)cc(C(F)(F)F)c1 ; Fc1cc(F)cc([S+](c2ccccc2)c2cc(F)cc(F)c2)c1 ; FC(F)(F)c1cccc(-[s+]2c3ccccc3c3ccccc32)c1]

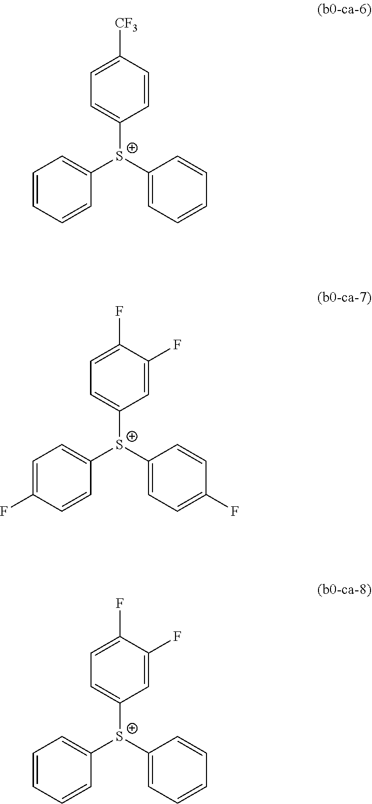 [SMILES: FC(F)(F)c1ccc([S+](c2ccccc2)c2ccccc2)cc1 ; Fc1ccc([S+](c2ccc(F)cc2)c2ccc(F)c(F)c2)cc1 ; Fc1ccc([S+](c2ccccc2)c2ccccc2)cc1F]

As the cation moiety of the component (B0), a cation represented by any of Formulae (b0-ca-1) to (b0-ca-8) is preferable, and a cation represented by any of Formulae (b0-ca-1) to (b0-ca-5) is more preferable.

In the present embodiment, it is preferable that the compound (B0) includes a compound (B01) represented by General Formula (b0-1).

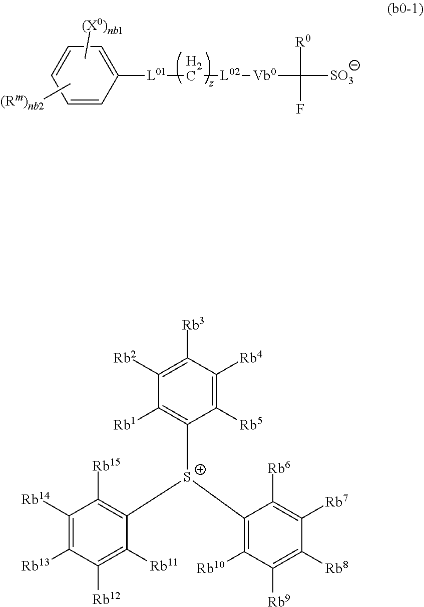

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰¹ and L⁰² each independently represent a single bond, an alkylene group, —O—, —CO—, —OCO—, —COO—, —SO₂—, —C(R^{b})═N—, or —CON(R^{b})—, R^{b} represents a hydrogen atom or an alkyl group, z represents an integer of 0 to 10, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7), Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group.]

X⁰, R^{m}, nb1, nb2, Vb⁰, R⁰, and Rb¹ to Rb¹⁵ in General Formula (b0-1) each have the same definition as that for X⁰, R^{m}, nb1, nb2, Vb⁰, R⁰, and Rb¹ to Rb¹⁵ in General Formula (b0).

L⁰¹, L⁰², and z in General Formula (b0-1) each have the same definition as that for L¹, L⁰², and z in General Formula (b0-an0).

Specific preferred examples of the component (B0) are shown below.

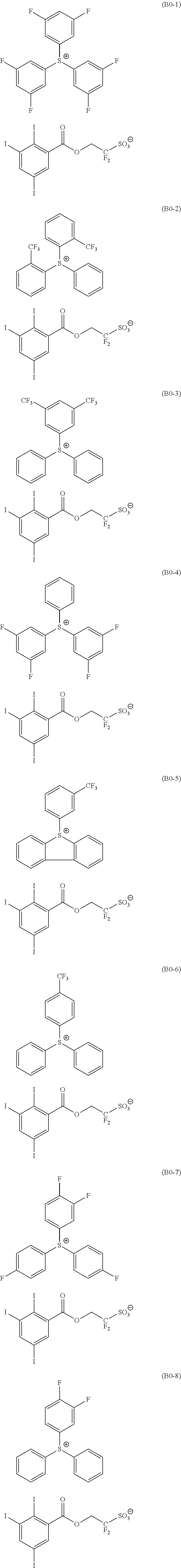

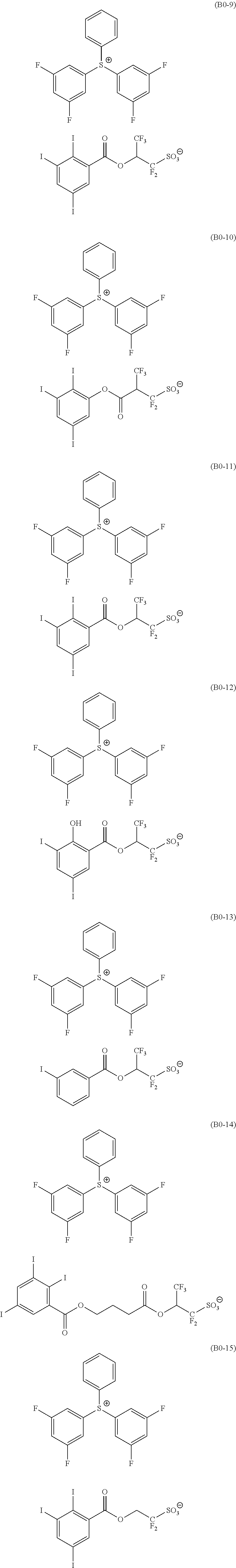

As the component (B0), a compound represented by any of Formulae (B0-1) to (B0-15) is preferable, and a compound represented by any one of Formulae (B0-1) to (B0-5) is more preferable.

In the resist composition according to the present embodiment, the component (B0) may be used alone or may be used in a combination of two or more kinds thereof.

In the resist composition according to the present embodiment, the amount of the component (B0) is preferably in a range of 5 to 40 parts by mass, more preferably in a range of 10 to 40 parts by mass, still more preferably in a range of 15 to 40 parts by mass, and particularly preferably in a range of 20 to 35 parts by mass with respect to 100 parts by mass of the component (A).

In a case where the amount of the component (B0) is greater than or equal to the lower limits of the above-described preferable ranges, the lithography characteristics such as the sensitivity, LWR, and the pattern shape in the resist pattern formation are further improved. Meanwhile, in a case where the content thereof is less than or equal to the upper limits of the above-described preferable ranges, a uniform solution is easily obtained, and the storage stability of the resist composition is further improved in a case of dissolving each component of the resist composition in an organic solvent.

The proportion of the component (B0) in the total component (B) contained in the resist composition according to the present embodiment is, for example, 50% by mass or greater, preferably 70% by mass or greater, and more preferably 95% by mass or greater. The proportion of the component (B0) in the total component (B) may be 100% by mass.

The component (B) in the resist composition according to the present embodiment may contain an acid generator component (B1) other than the above-described component (B0) (hereinafter, also referred to as “component (B1)”.

### <<Component (B1)>>

Examples of the component (B1) are numerous and include onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators; iminosulfonate-based acid generators; and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

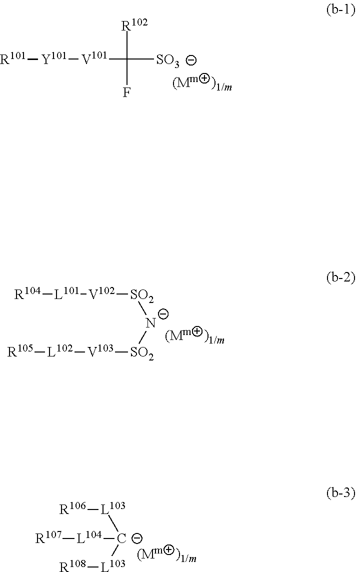

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M^{m+} represents an m-valent onium cation.]

{Anion moiety}
Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R¹⁰¹ include benzene, fluorene, naphthalene, anthracene, phenanthrene, biphenyl, or an aromatic heterocyclic ring in which some carbon atoms constituting these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclodecane, or tetracyclododecane; and a polycycloalkane having a fused ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰ may have a hetero atom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and other heterocyclic groups each represented by Chemical Formulae (r-hr-1) to (r-hr-16). In the formulae, * represents a bonding site with respect to Y¹⁰ in Formula (b-1).

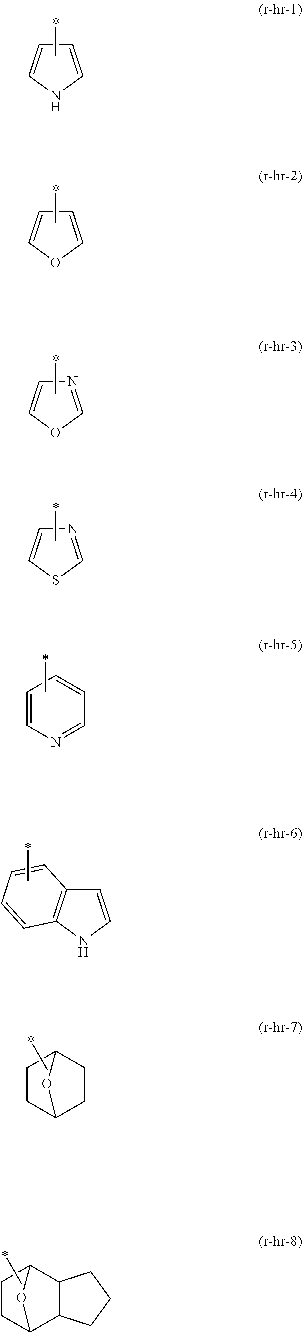

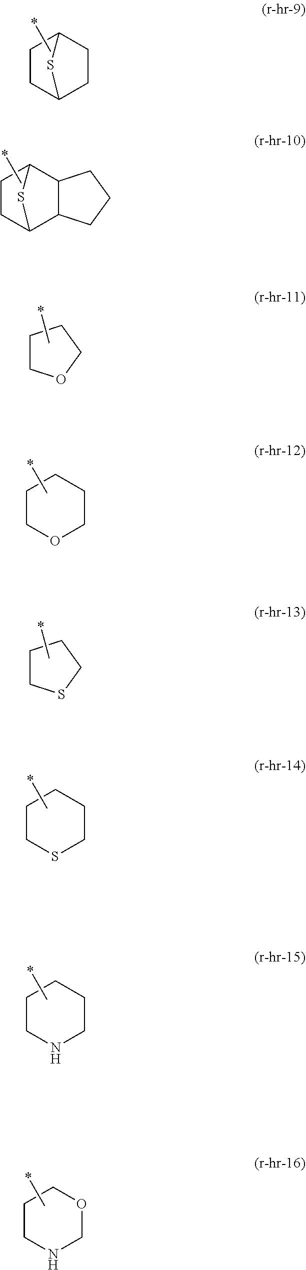

Examples of the substituent for the cyclic group as R¹⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom. Among these, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

The cyclic hydrocarbon group as R¹⁰¹ may be a fused cyclic group containing a fused ring in which an aliphatic hydrocarbon ring and an aromatic ring are fused. Examples of the fused ring include those obtained by fusing one or more aromatic rings with a polycycloalkane having a crosslinked ring-based polycyclic skeleton. Specific examples of the crosslinked ring-based polycycloalkane include a bicycloalkane such as bicyclo[2.2.1]heptane (norbornane) and bicyclo[2.2.2]octane. As the fused cyclic group, a group having a fused ring in which two or three aromatic rings are fused with a bicycloalkane is preferable, and a group having a fused ring in which two or three aromatic rings are fused with bicyclo[2.2.2]octane is more preferable. Specific examples of the fused cyclic group as R¹⁰¹ include those represented by Formulae (r-br-1) and (r-br-2). In the formulae, * represents a bonding site with respect to Y¹⁰¹ in Formula (b-1).

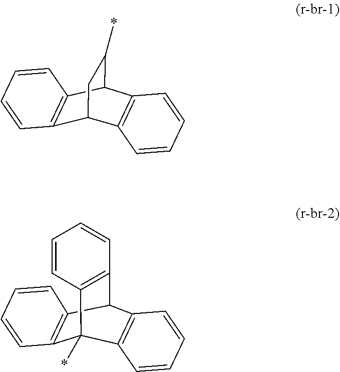 [SMILES: *C1CC2c3ccccc3C1c1ccccc12 ; *C12c3ccccc3C(c3ccccc31)c1ccccc12]

Examples of the substituent that the fused cyclic group as R10¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the fused cyclic group include those given as examples of the substituent of the cyclic group as R101.

Specific examples of the aromatic hydrocarbon group as the substituent of the fused cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the fused cyclic group include a group in which one hydrogen atom has been removed from a monocycloalkane such as cyclopentane or cyclohexane; a group in which one hydrogen atom has been removed from a polycycloalkane such as adamantane, norbornane, isobornane, tricyclodecane, or tetracyclododecane; a lactone-containing cyclic group represented by any of General Formulae (a2-r-1) to (a2-r-7); a —SO₂-containing cyclic group represented by any of General Formulae (a5-r-1) to (a5-r-4); and a heterocyclic group represented by any of Formulae (r-hr-7) to (r-hr-16).

Chain-like alkyl group which may have substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent:

The chain-like alkenyl group as R¹⁰ may be linear or branched, and the number of carbon atoms in the chain-like alkenyl group is preferably 2 to 10, more preferably 2 to 5, still more preferably 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ represents preferably a cyclic group which may have a substituent and more preferably a cyclic hydrocarbon group which may have a substituent. More specifically, as the cyclic hydrocarbon group, a phenyl group, a naphthyl group, or a group in which one or more hydrogen atoms have been removed from a polycycloalkane, a lactone-containing cyclic group represented by any of General Formulae (a2-r-1) to (a2-r-7), or a —SO₂-containing cyclic group represented by any of General Formulae (a5-r-1) to (a5-r-4) is preferable, a group in which one or more hydrogen atoms have been removed from a polycycloalkane or a —SO₂-containing cyclic group represented by any of General Formulae (a5-r-1) to (a5-r-4) is more preferable, and an adamantyl group or a —SO₂-containing cyclic group represented by General Formula (a5-r-1) is still more preferable.

In a case where the cyclic hydrocarbon group has a substituent, it is preferable that the substituent is a hydroxyl group.

In Formula (b-1), Y¹⁰¹ represents a single bond or a divalent linking group having an oxygen atom.

In a case where Y¹⁰¹ represents a divalent linking group containing an oxygen atom, Y¹⁰¹ may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination. Examples of the divalent linking group having an oxygen atom include linking groups each represented by Formulae (y-al-1) to (y-al-7). Further, in General Formulae (y-al-1) to (y-al-7), V′¹⁰¹ in General Formulae (y-al-1) to (y-al-7) is bonded to R¹⁰¹ in Formula (b-1).

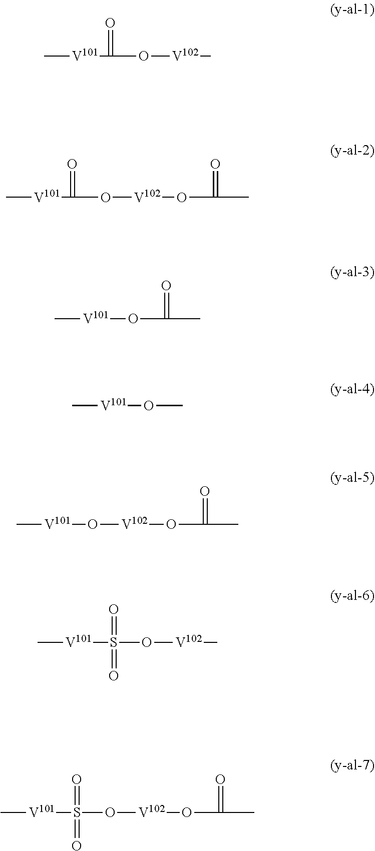

[In the formulae, V′¹⁰¹ represents a single bond or an alkylene group having 1 to 5 carbon atoms, and V′¹⁰² represents a divalent saturated hydrocarbon group having 1 to 30 carbon atoms.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, a part of methylene group in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been removed from the cyclic aliphatic hydrocarbon group (a monocyclic aliphatic hydrocarbon group or a polycyclic aliphatic hydrocarbon group) as Ra′³ in Formula (a1-r-1) is preferable, and a cyclohexylene group, a 1,5-adamantylene group, or a 2,6-adamantylene group is more preferable.

Y¹⁰¹ represents preferably a divalent linking group having an ester bond or a divalent linking group having an ether bond and more preferably a linking group represented by any of Formulae (y-al-1) to (y-al-5).

In Formula (b-1), V¹⁰¹ represents a single bond, an alkylene group, or a fluorinated alkylene group. It is preferable that the alkylene group and the fluorinated alkylene group as V101 have 1 to 4 carbon atoms. Examples of the fluorinated alkylene group as V¹⁰¹ include a group in which some or all hydrogen atoms in the alkylene group as V¹⁰¹ have been substituted with fluorine atoms. Among these examples, it is preferable that V¹⁰¹ represents a single bond or a fluorinated alkylene group having 1 to 4 carbon atoms.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

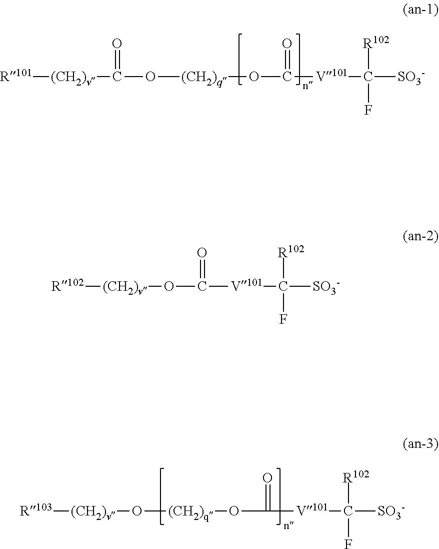

[In the formula, R″¹⁰¹ represents an aliphatic cyclic group which may have a substituent, a monovalent heterocyclic group represented by any of Chemical Formulae (r-hr-1) to (r-hr-6), a fused cyclic group represented by Formula (r-br-1) or (r-br-2), or a chain-like alkyl group which may have a substituent. R″¹⁰² represents an aliphatic cyclic group which may have a substituent, a fused cyclic group represented by General Formula (r-br-1) or (r-br-2), a lactone-containing cyclic group represented by any of General Formulae (a2-r-1) and (a2-r-3) to (a2-r-7), or a —SO₂-containing cyclic group represented by any of General Formulae (a5-r-1) to (a5-r-4). R″¹⁰³ represents an aromatic cyclic group which may have a substituent, an aliphatic cyclic group which may have a substituent, or a chain-like alkenyl group which may have a substituent. V″¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aromatic cyclic group as R″¹⁰³ which may have a substituent, the same groups as those for the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹⁰¹ in Formula (b-1) are preferable. As the chain-like alkenyl group as R″¹⁰³ which may have a substituent, the same groups as those for the chain-like alkenyl group as R¹⁰¹ in Formula (b-1) are preferable.

Anions in Component (b-2)

In Formula (b-2), R⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R¹⁰¹ in Formula (b-1). Here, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms because the solubility in a solvent for a resist is also satisfactory. Further, in the chain-like alkyl group as R¹⁰⁴ and R¹⁰⁵, it is preferable that the number of hydrogen atoms substituted with fluorine atoms is as large as possible because the acid strength increases and the transparency to high energy light or electron beams with a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V101 in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for Riot in Formula (b-1).

In Formula (b-3), L¹⁰³ to L¹⁰⁵ each independently represent a single bond, —CO—, or —SO₂—.

Among the examples, as the anion moiety of the component (B), an anion in the component (b-1) is preferable. Among the examples, an anion represented by any of General Formulae (an-1) to (an-3) is more preferable, an anion represented by General Formula (an-1) or (an-2) is still more preferable, and an anion represented by General Formula (an-2) is particularly preferable.

### {Cation Moiety}

In Formulae (b-1), (b-2), and (b-3), M^{m+} represents an m-valent onium cation. Among these, a sulfonium cation and an iodonium cation are preferable.

m represents an integer of 1 or greater.

Preferred examples of the cation moiety ((M^{m+})_{l/m}) include organic cations each represented by General Formulae (ca-1) to (ca-5).

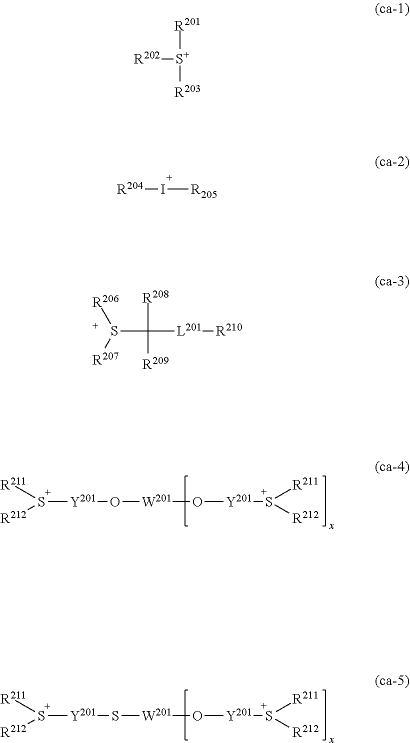

[In the formulae, R²⁰¹ to R²⁰⁷, R²¹¹, and R²¹² each independently represent an aryl group, an alkyl group, or an alkenyl group which may have a substituent. R²⁰¹ to R²⁰³, R²⁰⁶ and R²⁰⁷, and R²¹¹ and R²¹² may be bonded to each other to form a ring with the sulfur atom in the formula. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—. Y²⁰¹'s each independently represent an arylene group, an alkylene group, or an alkenylene group. x represents 1 or 2. W²⁰¹ represents an (x+1)-valent linking group.]

In General Formulae (ca-1) to (ca-5), examples of the aryl group as R²⁰¹ to R²⁰⁷, R²¹¹, and R²¹² include an unsubstituted aryl group having 6 to 20 carbon atoms. Among these, a phenyl group or a naphthyl group is preferable.

The alkyl group as R²⁰¹ to R²⁰⁷, R²¹¹, and R²¹² is a chain-like or cyclic alkyl group, and the number of carbon atoms thereof is preferably in a range of 1 to 30.

It is preferable that the alkenyl group as R²⁰¹ to R²⁰⁷, R²¹¹, and R²¹² has 2 to 10 carbon atoms.

Examples of the substituent which may be included in R²⁰¹ to R²⁰⁷ and R²¹¹ to R²¹² include an alkyl group, a halogen atom, a halogenated alkyl group, a carbonyl group, a cyano group, an amino group, an aryl group, and groups each represented by General Formulae (ca-r-1) to (ca-r-7).

In General Formulae (ca-1) to (ca-5), in a case where R²⁰¹ to R²⁰³, R²⁰⁶ and R²⁰⁷, and R²¹1 and R²¹² are bonded to each other to form a ring with a sulfur atom in the formula, these groups may be bonded to each other via a hetero atom such as a sulfur atom, an oxygen atom, or a nitrogen atom, or a functional group such as a carbonyl group, —SO—, —SO₂—, —SO₃—, —COO—, —CONH— or —N(R_{N})— (here, R_{N} represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ to represents an aryl group which may have a substituent, an alkyl group which may have a substituent, an alkenyl group which may have a substituent, or a —SO₂— containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹⁰, a chain-like or cyclic alkyl group having 1 to 30 carbon atoms is preferable.

It is preferable that the alkenyl group as R²¹⁰ has 2 to 10 carbon atoms. As the —SO₂-containing cyclic group as R²¹⁰ which may have a substituent, “—SO₂-containing polycyclic group” is preferable, and a group represented by General Formula (a5-r-1) is more preferable.

Y²⁰¹'s each independently represent an arylene group, an alkylene group, or an alkenylene group.

Examples of the arylene group as Y²⁰¹ include a group in which one hydrogen atom has been removed from an aryl group which is given as an example of the aromatic hydrocarbon group represented by R¹⁰¹ in Formula (b-1).

Examples of the alkylene group and alkenylene group as Y²⁰¹ include a group in which one hydrogen atom has been removed from the chain-like alkyl group or the chain-like alkenyl group as R¹⁰¹ in Formula (b-1).

In Formula (ca-4), x represents 1 or 2.

W²⁰¹ represents an (x+1)-valent linking group, that is, a divalent or trivalent linking group.

As the divalent linking group represented by W²⁰¹, a divalent hydrocarbon group which may have a substituent is preferable, and examples thereof include the same divalent hydrocarbon groups which may have a substituent as those for Ya²¹ in General Formula (a2-1). The divalent linking group as W201 may be any of linear, branched, or cyclic and is preferably cyclic. Among these, a group in which two carbonyl groups are combined with both ends of the arylene group is preferable. Examples of the arylene group include a phenylene group and a naphthylene group. Among these, a phenylene group is particularly preferable.

Examples of the trivalent linking group as W²⁰¹ include a group obtained by removing one hydrogen atom from the above-described divalent linking group as W²⁰¹ and a group obtained by bonding the divalent linking group to another divalent linking group described above. As the trivalent linking group as W⁰, a group obtained by bonding two carbonyl groups to an arylene group is preferable.

Specific examples of the suitable cation represented by Formula (ca-1) include cations each represented by Chemical Formulae (ca-1-1) to (ca-1-72) shown below.

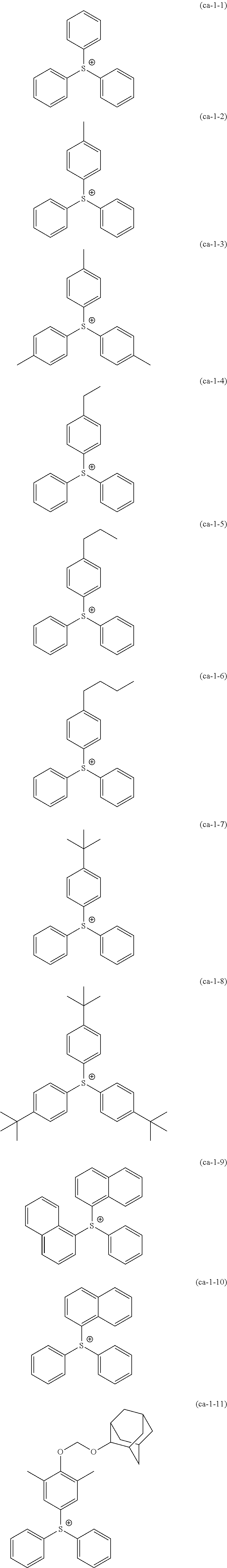

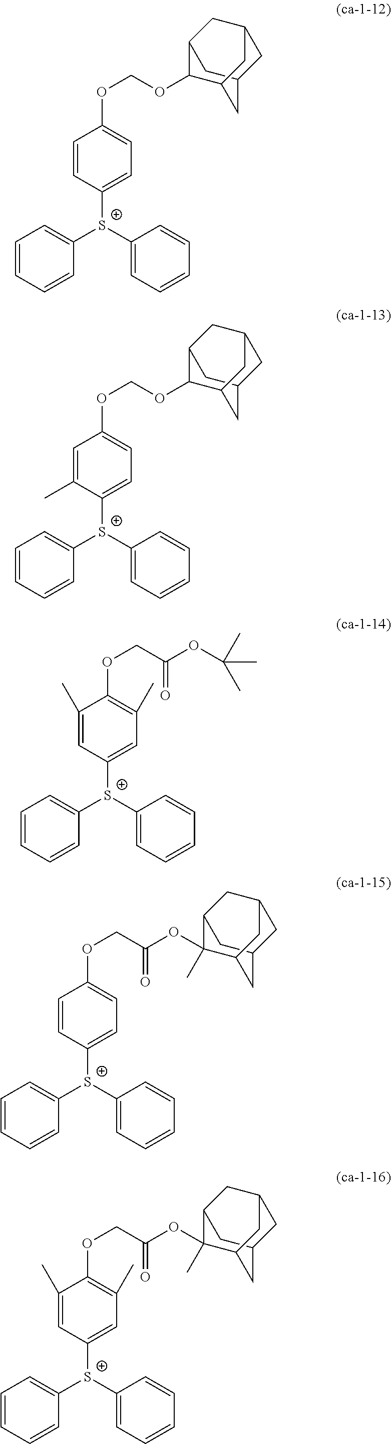 [SMILES: c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(=O)OC(C)(C)C ; CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3]

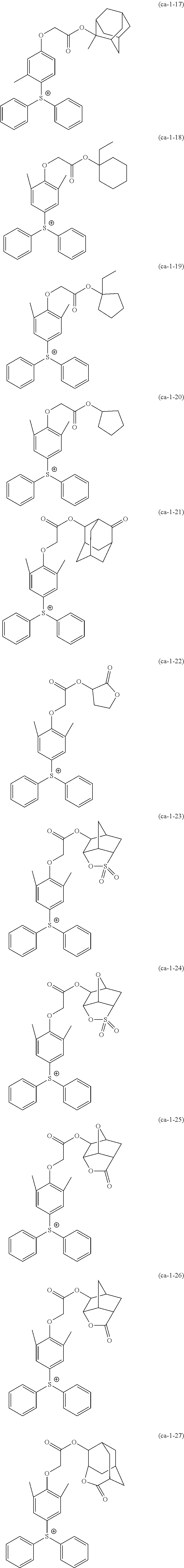

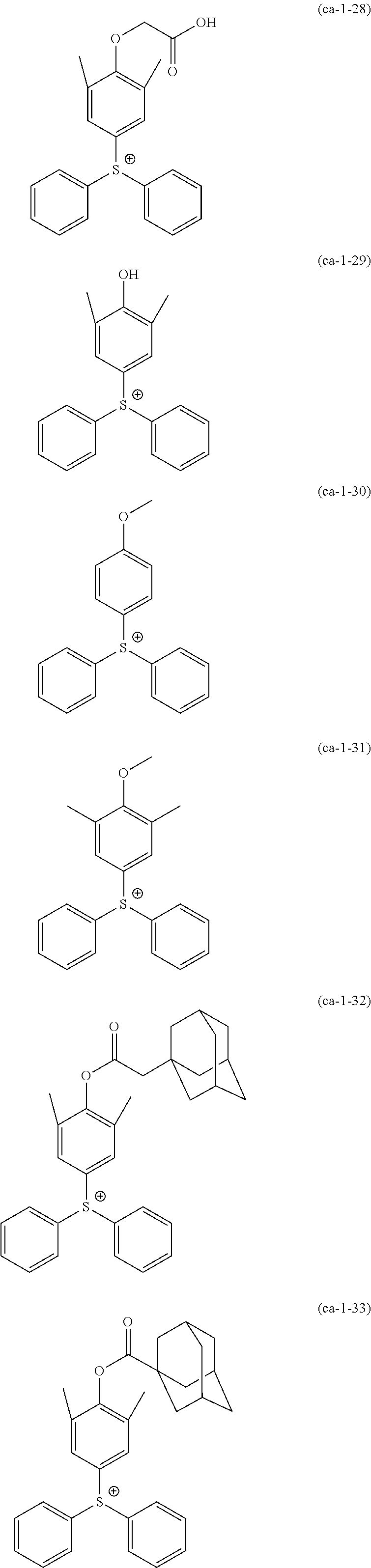 [SMILES: Cc1cc([S+](c2ccccc2)c2ccccc2)cc(C)c1OCC(=O)O ; Cc1cc([S+](c2ccccc2)c2ccccc2)cc(C)c1O ; COc1ccc([S+](c2ccccc2)c2ccccc2)cc1 ; COc1c(C)cc([S+](c2ccccc2)c2ccccc2)cc1C ; Cc1cc([S+](c2ccccc2)c2ccccc2)cc(C)c1OC(=O)CC12CC3CC(CC(C3)C1)C2 ; Cc1cc([S+](c2ccccc2)c2ccccc2)cc(C)c1OC(=O)C12CC3CC(CC(C3)C1)C2]

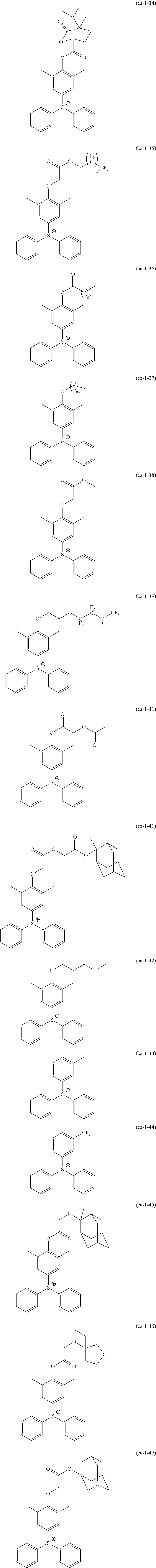

[In the formulae, g1, g2, and g3 represent a repeating number, g1 represents an integer of 1 to 5, g2 represents an integer of 0 to 20, and g3 represents an integer of 0 to 20.]

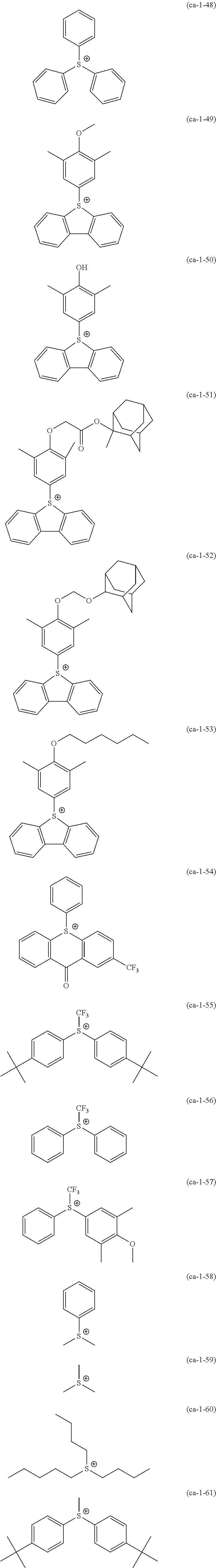

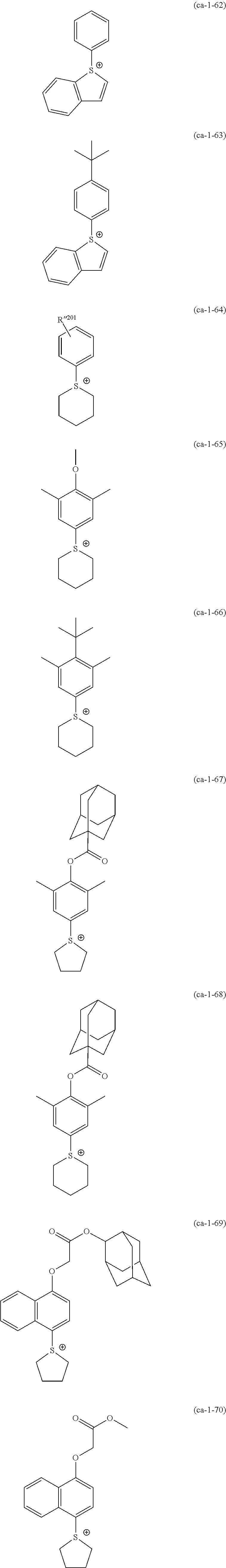

[In the formulae, R″ ²⁰¹ represents a hydrogen atom or a substituent, and examples of the substituent include the same groups as those for the substituents which may be included in R²⁰¹ to R²⁰⁷ and R²¹⁰ to R²¹²

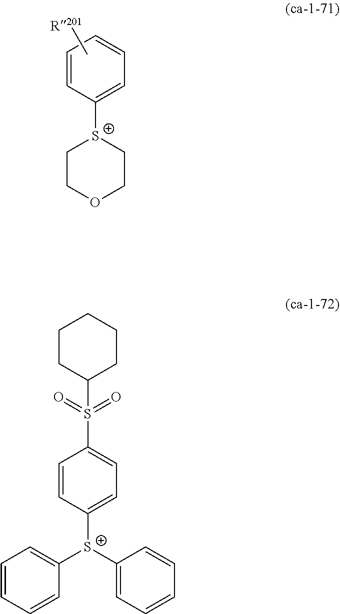

Specific examples of suitable cations represented by Formula (ca-2) include a diphenyliodonium cation and a bis(4-tert-butylphenyl)iodonium cation.

Specific examples of suitable cations represented by Formula (ca-3) include cations each represented by Formulae (ca-3-1) to (ca-3-6).

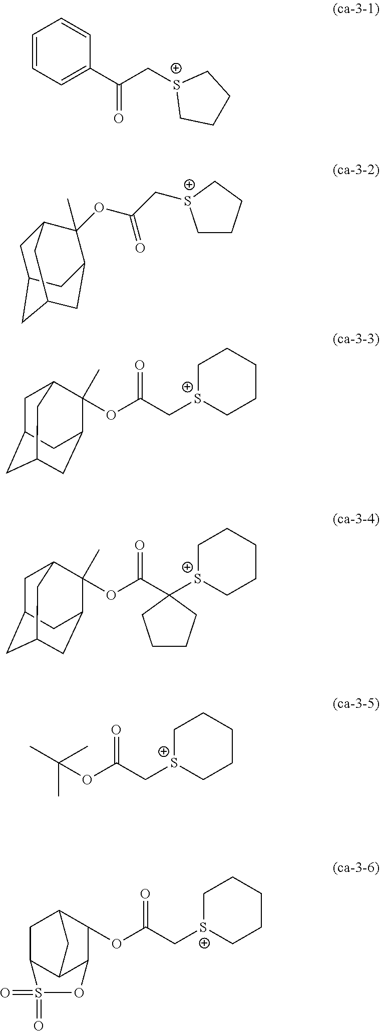 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

Specific examples of suitable cations represented by Formula (ca-4) include cations each represented by Formulae (ca-4-1) and (ca-4-2).

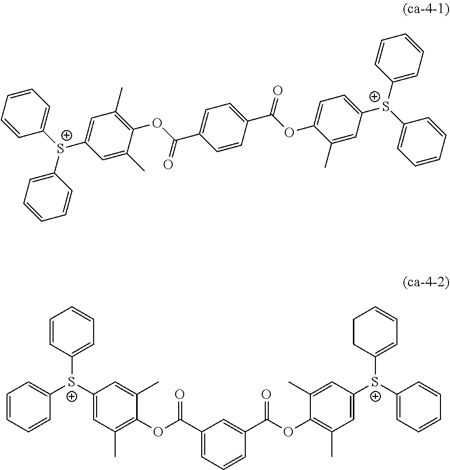 [SMILES: Cc1cc([S+](c2ccccc2)c2ccccc2)ccc1OC(=O)c1ccc(C(=O)Oc2c(C)cc([S+](c3ccccc3)c3ccccc3)cc2C)cc1 ; Cc1cc([S+](C2=CC=CCC2)c2ccccc2)cc(C)c1OC(=O)c1cccc(C(=O)Oc2c(C)cc([S+](c3ccccc3)c3ccccc3)cc2C)c1]

Specific examples of suitable cations represented by Formula (ca-5) include cations each represented by General Formulae (ca-S—1) and (ca-S—31.

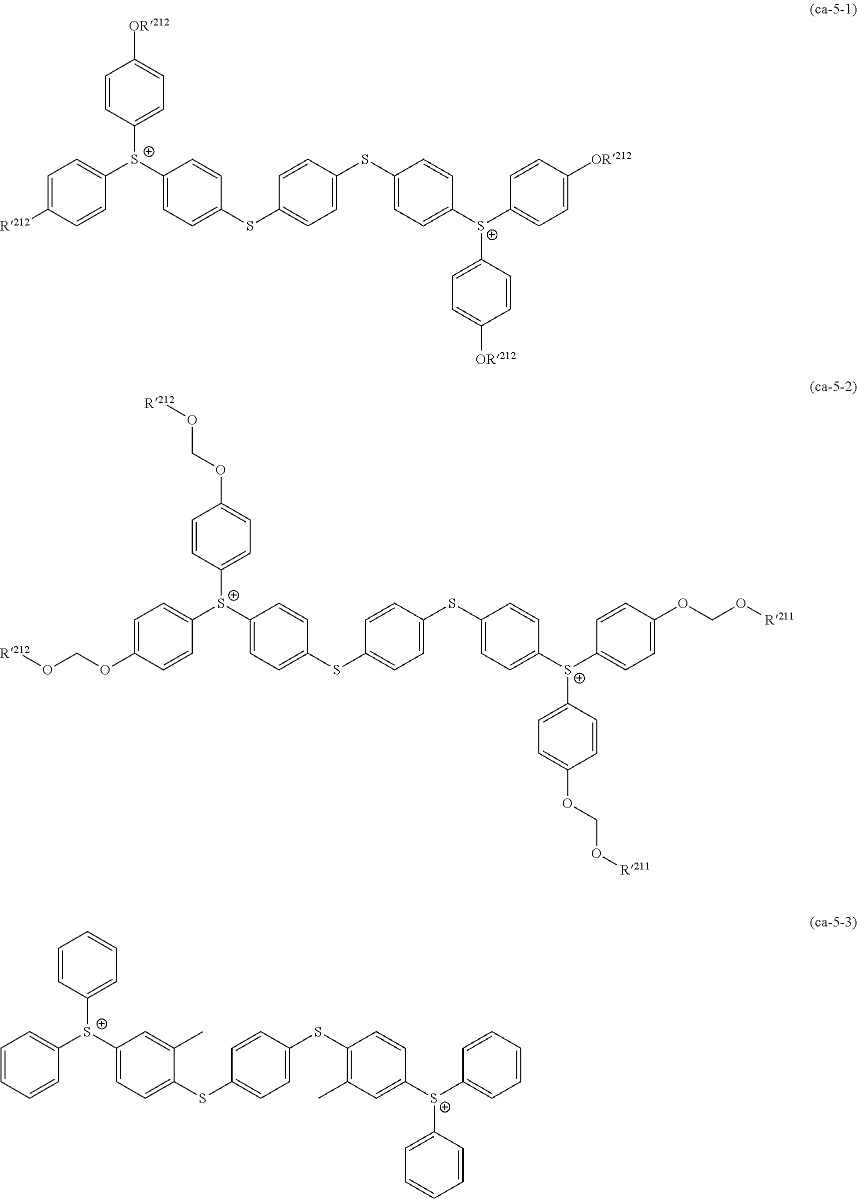

Among the examples, as the cation moiety ((M^{m+})_{t/m}), a cation represented by 5 General Formula (ca-1) is preferable.

In the resist composition according to the present embodiment, the component (B) may be used alone or a combination of two or more kinds thereof may be used.

The amount of the component (B) in the resist composition of the present embodiment is preferably less than 40 parts by mass, more preferably in a range of 1 to 30 parts by mass, and still more preferably in a range of 3 to 25 parts by mass with respect to 100 parts by mass of the component (A).

In a case where the amount of the component (B) is set to be in the above-described preferable range, pattern formation can be satisfactorily performed. Further, it is preferable that each component of the resist composition is dissolved in an organic solvent from the viewpoint that a uniform solution is easily obtained and the storage stability of the resist composition is improved.

It is preferable that the resist composition according to the present embodiment does not contain the component (B1).

### <Other Components>

The resist composition of the present embodiment may further contain other components in addition to the component (A) and the component (B) described above. Examples of other components include a component (D), a component (E), a component (F), and a component (S) described below.

<<Base component (D)>>

The resist composition of the present embodiment may further contain, in addition to the component (A), a base component (component (D)) that traps an acid generated upon light exposure (that is, controls diffusion of an acid). The component (D) acts as a quencher (an acid diffusion control agent) which traps the acid generated in the resist composition upon light exposure.

Examples of the component (D) include a photodecomposable base (D1) having an acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (component (D1)) is preferable from the viewpoint of easily increasing the sensitivity, reducing the roughness, and improving the characteristic of suppressing occurrence of coating defects.

In regard to component (D1)

In a case where a resist composition containing the component (D1) is obtained, the contrast between an exposed portion and an unexposed portion of the resist film can be further improved in a case of forming a resist pattern.

The component (D1) is not particularly limited as long as the component is decomposed upon light exposure and loses acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable, and the component (d1-1) is more preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film.

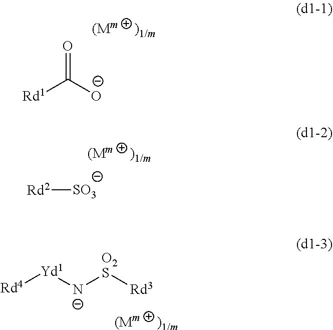

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom in Rd² in Formula (d1-2) has no fluorine atom bonded thereto. Yd¹ represents a single bond or a divalent linking group. m represents an integer of 1 or greater, and M^{m+}'s each independently represents an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-al-1) to (y-al-5) is preferable as the substituent. Further, in a case where the aromatic hydrocarbon group, the aliphatic cyclic group, or the chain-like alkyl group as Rd¹ contain a linking group represented by any of Formulae (y-al-1) to (y-al-7) as a substituent, V′¹⁰¹ in Formula (y-al-1) to (y-al-7) is bonded to the carbon atom constituting the aromatic hydrocarbon group, the aliphatic cyclic group, or the chain-like alkyl group as Rd¹ in Formula (d3-1), in General Formulae (y-al-1) to (y-al-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclodecane or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

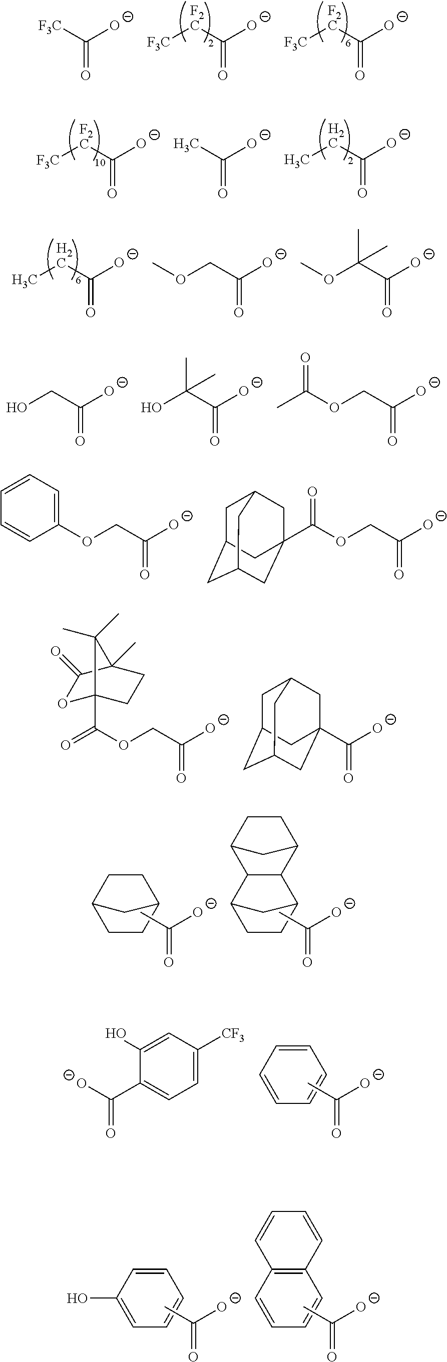

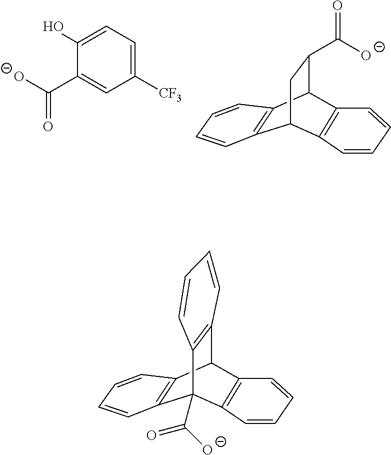 [SMILES: O=C([O-])c1cc(C(F)(F)F)ccc1O ; O=C([O-])C1CC2c3ccccc3C1c1ccccc12 ; O=C([O-])C12c3ccccc3C(c3ccccc31)c1ccccc12]

### Cation Moiety

In Formula (d1-1), M^{m+} represents an m-valent organic cation.

Examples of the organic cation as M^{m+} include those for the cations represented by General Formulae (ca-1) to (ca-5). Among these, the cation represented by General Formula (ca-1) is more preferable, and the cations each represented by General Formulae (ca-1-1) to (ca-1-72) are still more preferable.

The component (d1-1) may be used alone or a combination of two or more kinds thereof may be used.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom in Rd² has no fluorine atom bonded thereto (the carbon atom is not substituted with a fluorine atom). In this manner, the anion of the component (d1-2) is an appropriately weak acid anion, thereby improving the quenching ability of the component (D).

It is preferable that Rd² represents a chain-like alkyl group which may have a substituent or an aliphatic cyclic group which may have a substituent. The chain-like alkyl group has preferably 1 to 10 carbon atoms and more preferably 3 to 10 carbon atoms. As the aliphatic cyclic group, a group in which one or more hydrogen atoms have been removed from adamantane, norbornane, isobornane, tricyclodecane, or tetracyclododecane (a group which may have a substituent); and a group in which one or more hydrogen atoms have been removed from camphor are more preferable.

The hydrocarbon group as Rd² may have a substituent, and examples of the substituent include the same groups as those for the substituent which may be included in the hydrocarbon group (such as an aromatic hydrocarbon group, an aliphatic cyclic group, or a chain-like alkyl group) as Rd¹ in Formula (d1-1).

Specific preferred examples of the anion moiety in the component (d1-2) are described below.

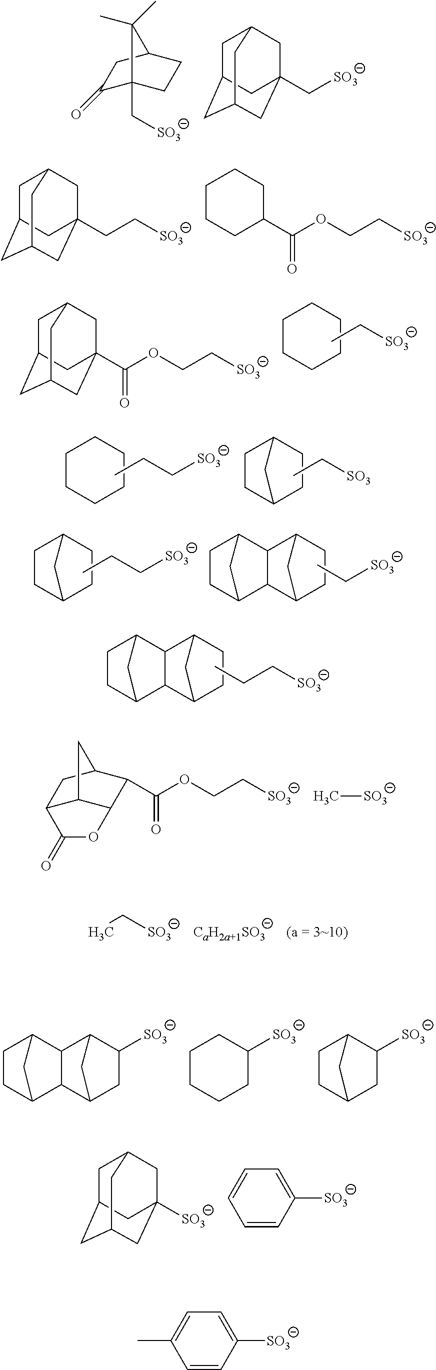

### Cation Moiety

In Formula (d1-2), M^{m+} represents an m-valent organic cation and has the same definition as that for M^{m+} in Formula (d1-1).

The component (d1-2) may be used alone or a combination of two or more kinds thereof may be used.

{Component (d1-3)}

### Anion Moiety

In Formula (d1-3), Rd¹ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are improved. Further, in a case where Rd⁴ represents an aromatic group, the resist composition has excellent light absorption efficiency in lithography using EUV or the like as an exposure light source, and thus the sensitivity and lithography characteristics are improved.

In Formula (d1-3), Yd¹ represents a single bond or a divalent linking group. The divalent linking group as Yd¹ is not particularly limited, and examples thereof include a divalent hydrocarbon group (an aliphatic hydrocarbon group or an aromatic hydrocarbon group) which may have a substituent and a divalent linking group having a hetero atom. These divalent linking groups are the same as those for the divalent hydrocarbon group which may have a substituent and the divalent linking group having a hetero atom described in the section of the divalent linking group as Ya²¹ in Formula (a2-1).

It is preferable that Yd¹ represents a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

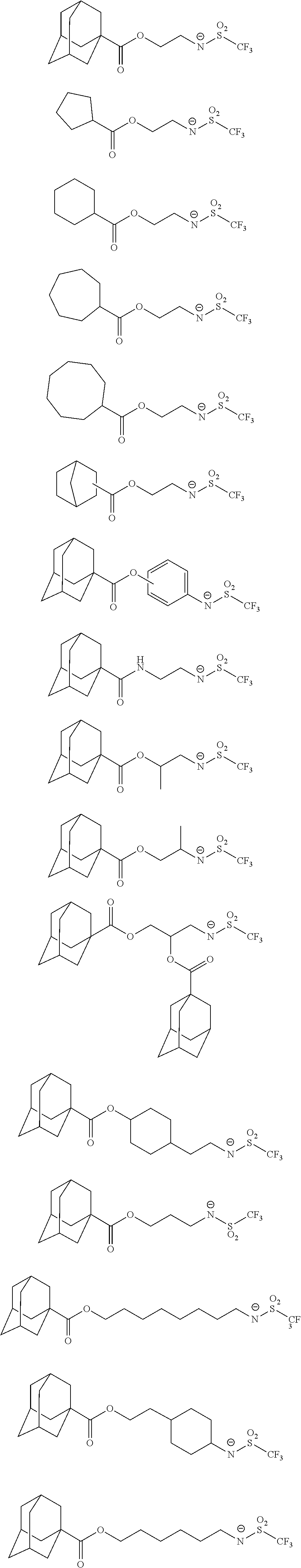

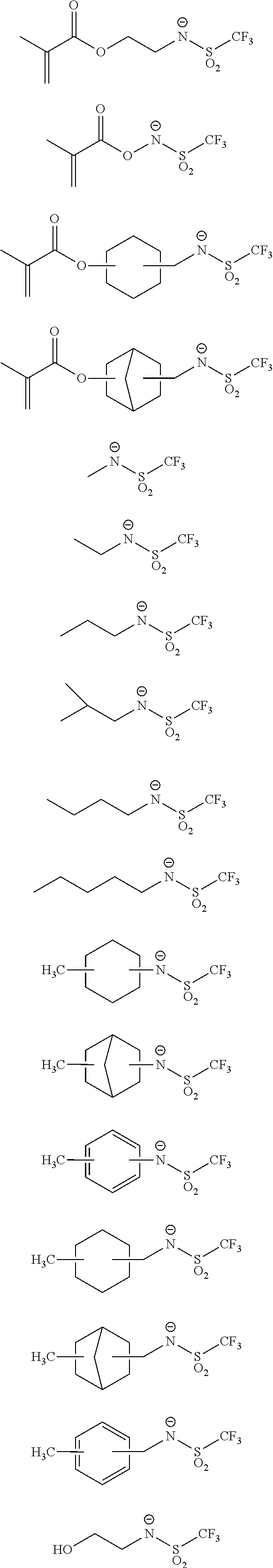

### Cation Moiety

In Formula (d1-3), M^{m+} represents an m-valent organic cation and has the same definition as that for M^{m+} in Formula (d1-1).

The component (d1-3) may be used alone or a combination of two or more kinds thereof may be used.

As the component (D1), only one of the above-described components (d1-1) to (d1-3) or a combination of two or more kinds thereof may be used.

In a case where the resist composition contains the component (D1), the amount of the component (D1) in the resist composition is preferably in a range of 0.5 to 20 parts by mass, more preferably in a range of 1 to 15 parts by mass, and still more preferably in a range of 2 to 8 parts by mass with respect to 100 parts by mass of the component (A1).

In a case where the amount of the component (D1) is greater than or equal to the lower limits of the above-described ranges, particularly excellent lithography characteristics and an excellent resist pattern shape are easily obtained. On the contrary, in a case where the content is less than or equal to the upper limits of the above-described ranges, the sensitivity can be satisfactorily maintained and the throughput is also excellent.

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

In regard to component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an anine containing one or more aliphatic groups, and the number of carbon atoms in the aliphatic group is preferably in a range of 1 to 12.

Examples of the aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylanines such as n-hexylamine, n-heptylanine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolanine, triisopropanolamine, di-n-octanolamine, and tri-n-octanolamine. Among these, a trialkylamine having 5 to 10 carbon atoms is preferable, and tri-n-pentylamine and tri-n-octylamine are particularly preferable.

Examples of the cyclic amine include a heterocyclic compound having a nitrogen atom as a hetero atom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1, 5-diazabicyclo[4.3.0]—S— nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris {2-(2-methoxyethoxy)ethyl}amine, tris {2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxyl}ethyl]amine, and triethanolamine triacetate. Among these, triethanolamine triacetate is preferable.

As the component (D2), an aromatic amine may be used.

Examples of the aromatic amine include 4-dimethylaminopyridine, pyrrole, indole, pyrazole, imidazole, and derivatives thereof, tribenzylamine, 2,6-diisopropylaniline, and N-tert-butoxycarbonylpyrrolidine.

The component (D2) may be used alone or a combination of two or more kinds thereof may be used.

In a case where the resist composition contains the component (D2), the amount of the component (D2) in the resist composition is typically in a range of 0.01 to 5 parts by mass with respect to 100 parts by mass of the component (A1). In a case where the content thereof is set to be in the above-described range, the resist pattern shape, the post exposure temporal stability, and the like are improved.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivatives include phosphoric acid esters such as phosphoric acid di-n-butyl ester and phosphoric acid diphenyl ester.

Examples of the phosphonic acid derivatives include phosphonic acid esters such as phosphonic acid dimethyl ester, phosphonic acid di-n-butyl ester, phenylphosphonic acid, phosphonic acid diphenyl ester, and phosphonic acid dibenzyl ester.

Examples of the phosphinic acid derivatives include phosphinic acid ester and phenylphosphinic acid.

In the resist composition of the present embodiment, the component (E) may be used alone or a combination of two or more kinds thereof may be used.

In a case where the resist composition contains the component (E), the amount of the component (E) is preferably in a range of 0.01 to 5 parts by mass and more preferably in a range of 0.05 to 3 parts by mass with respect to 100 parts by mass of the component (A). In a case where the content thereof is in the above-described range, the sensitivity, lithography characteristics, and the like are improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by Formula (f1-1). As the polymer, a polymer (homopolymer) formed of only the constitutional unit (f1) represented by Formula (f1-1); a copolymer of the constitutional unit (f]) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

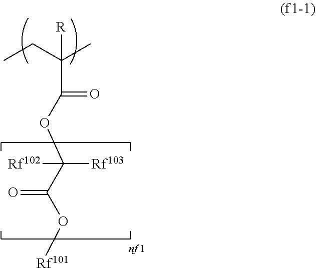

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰²² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or more of the hydrogen atoms in the hydrocarbon group are fluorinated, more preferably 50% or more thereof are fluorinated, and still more preferably 60% or more thereof are fluorinated from the viewpoint of increasing the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or a combination of two or more kinds thereof may be used.

In a case where the resist composition contains the component (F), the amount of the component (F) is preferably in a range of 0.5 to 10 parts by mass and more preferably in a range of 1 to 10 parts by mass with respect to 100 parts by mass of the component (A).

<<Organic solvent component (S)>>

The resist composition of the present embodiment can be produced by dissolving the resist materials in an organic solvent component (hereinafter, referred to as “component (S)”).

The component (S) may be any organic solvent which can dissolve the each component to be used to obtain a uniform solution, and an optional organic solvent can be appropriately selected from those which have been known as solvents of a chemically amplified resist composition and then used.

Examples of the component (S) include lactones such as y-butyrolactone; ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond, such as ethylene glycol monoacetate, diethylene glycol monoacetate, propylene glycol monoacetate, and dipropylene glycol monoacetate; polyhydric alcohol derivatives of compounds having an ether bond such as monoalkyl ether or monophenyl ether, such as monomethylether, monoethylether, monopropylether, or monobutylether of polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylmethylether, diphenylether, dibenzylether, phenetole, butylphenylether, ethylbenzene, diethylbenzene, pentylbenzene, isopropylbenzene, toluene, xylene, cymene, and mesitylene; and dimethylsulfoxide (DMSO).

In the resist composition of the present embodiment, the component (S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component (S). The blending ratio (mass ratio) of the mixed solvent can be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. Further, in a case where PGME is blended as the polar solvent, the mass ratio of PGMEA to PGME is preferably in a range of 1:9 to 9:1, more preferably in a range of 2:8 to 8:2, and still more preferably in a range of 3:7 to 7:3. Further, a mixed solvent of PGMEA, PGME, and cyclohexanone is also preferable.

Further, a mixed solvent of γ-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

As desired, miscible additives such as additive resins, dissolution inhibitors, plasticizers, stabilizers, colorants, halation prevention agents, and dyes for improving the performance of the resist film can be added to the resist composition of the present embodiment, as appropriate.

After the resist material is dissolved in the component (S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition according to the present embodiment described above contains the resin component (A1) and the compound (B0) (component (B0)).

The component (B0) has an iodine atom having a high absorption cross-sectional area in the anion moiety with respect to EUV and EB. Therefore, a large amount of secondary electrons can be generated by improving the sensitivity to EUV and EB as compared with an acid generator having no iodine atom in the related art. In addition, the component (B0) contains a fluorine atom in the cation moiety and thus has a high ability to supplement secondary electrons. Therefore, in the component (B0), the amount of the acid generated upon light exposure increases due to the synergistic effect of the anion moiety and the cation moiety. As a result, according to the resist composition of the present embodiment, it is assumed that a resist pattern having high sensitivity and satisfactory lithography characteristics such as LWR can be formed.

Further, since the amount of the acid generated upon light exposure in the component (B0) is large, the deprotection of the acid dissociable group further proceeds in the exposed portions, the solubility in an alkali developing solution is improved, and the solubility in the developing solution containing an organic solvent decreases. Meanwhile, the component (B0) has iodine atoms in the anion moiety and fluorine atoms in the cation moiety, and thus is highly hydrophobic. Therefore, it is assumed that in a case where the deprotection of the acid dissociable group further proceeds in the exposed portions, the dissolution contrast between the exposed portions and the unexposed portions is improved, and thus a resist pattern having high rectangularity can be formed.

### (Resist Pattern Formation Method According to Second Aspect of Present Invention)

A resist pattern formation method according to the second aspect according to the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method by performing processes as described below is an exemplary example.

First, a support is coated with the resist composition of the present embodiment using a spinner or the like, and a bake (post applied bake (PAB)) treatment is performed under a temperature condition of 80° C. to 150° C. for 40 to 120 seconds and preferably 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing with an electron beam without using a mask pattern, baking treatment (post-exposure baking (PEB)) is carried out, for example, under a temperature condition in a range of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

Further, as the support, any one of the above-described supports provided with an inorganic and/or organic film on the above-described substrate may be used. As the inorganic film, an inorganic antireflection film (inorganic BARC) can be used. As the organic film, an organic film such as an organic antireflection film (organic BARC) or a lower-layer organic film used in a multilayer resist method can be used.

Here, the multilayer resist method is a method of providing at least one layer of an organic film (lower-layer organic film) and at least one layer of a resist film (upper-layer resist film) on a substrate and performing patterning of the lower-layer organic film using a resist pattern formed on the upper-layer resist film as a mask, and this method is considered to enable formation of a pattern with a high aspect ratio. That is, according to the multilayer resist method, since a desired thickness can be ensured by the lower-layer organic film, the thickness of the resist film can be reduced, and a fine pattern with a high aspect ratio can be formed.

The multilayer resist method is basically classified into a method in which a double-layer structure consisting of an upper-layer resist film and a lower-layer organic film is formed (two-layer resist method), and a method in which a multilayer structure having at least three layers consisting of an upper-layer resist film, a lower-layer organic film, and at least one intermediate layer (thin metal film or the like) provided between the upper-layer resist film and the lower-layer organic film (three-layer resist method).

The wavelength to be used for light exposure is not particularly limited and the exposure can be conducted using radiation such as an ArF excimer laser, a KrF excimer laser, an F₂ excimer laser, extreme ultraviolet rays (EUV), vacuum ultraviolet rays (VUV), electron beams (EB), X-rays, and soft X-rays. The resist composition is useful for a KrF excimer laser, an ArF excimer laser, EB, and EUV, more useful for an ArF excimer laser, EB, and EUV, and particularly useful for EB and EUV. That is, the resist pattern formation method according to the present embodiment is a method particularly useful in a case where the step of exposing the resist film to light includes a process of exposing the resist film to extreme ultraviolet (EUV) rays or electron beams (EB).

The method of exposing the resist film to light may be typical exposure (dry exposure) conducted in air or an inert gas such as nitrogen, or liquid immersion exposure (liquid immersion lithography). Among these, liquid immersion exposure is preferable.

The liquid immersion exposure is an exposure method in which the region between the resist film and the lens at the lowermost position of the exposure apparatus is filled with a solvent (liquid immersion medium) in advance that has a larger refractive index than the refractive index of air, and the exposure (immersion exposure) is conducted in this state.

As the liquid immersion medium, a solvent which has a refractive index larger than the refractive index of air but smaller than the refractive index of the resist film to be exposed to light is preferable. The refractive index of such a medium is not particularly limited as long as the refractive index is in the above-described range.

Examples of the solvent which has a refractive index that is larger than the refractive index of air but smaller than the refractive index of the resist film include water, a fluorine-based inert liquid, a silicon-based solvent, and a hydrocarbon-based solvent.

Specific examples of the fluorine-based inert liquid include a liquid containing a fluorine-based compound such as C₃HCl₂F₅, C₄F₉OCH₃, C₄F₉OC₂H₅, or C₅H₃F₇ as a main component, and a liquid with a boiling point of 70° C. to 180° C. is preferable and a liquid with a boiling point of 80° C. to 160° C. is more preferable. A fluorine-based inert liquid having a boiling point in the above-described range is preferable from the viewpoint that a medium used for liquid immersion can be removed using a simple method after completion of light exposure.

As the fluorine-based inert liquid, a perfluoroalkyl compound in which all hydrogen atoms in the alkyl group have been substituted with fluorine atoms is particularly preferable. Specific examples of the perfluoroalkyl compound include a perfluoroalkylether compound and a perfluoroalkylamine compound.

Further, specific examples of the perfluoroalkylether compound include perfluoro(2-butyl-tetrahydrofuran) (boiling point of 102° C.), and specific examples of the perfluoroalkylamine compound include perfluorotributylamine (boiling point of 174° C.).

As the liquid immersion medium, water is preferable from the viewpoints of the cost, the safety, the environmental issues, and the versatility.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

The ketone-based solvent is an organic solvent containing C—C(═O)—C in the structure thereof. The ester-based solvent is an organic solvent containing C—C(═O)—O—C in the structure thereof. The alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group. The nitrile-based solvent is an organic solvent containing a nitrile group in the structure thereof. The amide-based solvent is an organic solvent containing an amide group in the structure thereof. The ether-based solvent is an organic solvent containing C—O—C in the structure thereof.

Some organic solvents have a plurality of the functional groups which characterize each of the solvents in the structure thereof. In such a case, the organic solvents are considered to correspond to all the solvents containing the functional groups. For example, diethylene glycol monomethylether corresponds to both the alcohol-based solvent and the ether-based solvent which have been classified above.

The hydrocarbon-based solvent is a hydrocarbon solvent which is formed of a hydrocarbon that may be halogenated and does not have a substituent other than halogen atoms. Among these, a fluorine atom is preferable as the halogen atom.

Among the examples, as the organic solvent contained in the organic developing solution, a polar solvent is preferable. Further, a ketone solvent, an ester solvent, and a nitrile solvent are preferable.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonylalcohol, acetylcarbinol, acetophenone, methyl naphthyl ketone, isophorone, propylenecarbonate, γ-butyrolactone, and methyl amyl ketone (2-heptanone). Among these examples, methyl amyl ketone (2-heptanone) is preferable as the ketone-based solvent.

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate, and propyl-3-methoxypropionate. Among these examples, butyl acetate is preferable as the ester-based solvent.

Examples of the nitrile-based solvent include acetonitrile, propionitrile, valeronitrile, and butyronitrile.

Known additives can be blended into the organic developing solution as desired. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents which are given as examples of the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one solvent selected from a hydrocarbon-based solvent, a ketone-based solvent, an ester-based solvent, an alcohol-based solvent, and an amide-based solvent is preferable, at least one solvent selected from an alcohol-based solvent and an ester-based solvent is more preferable, and an alcohol-based solvent is particularly preferable.

As the alcohol-based solvent used in the rinse solution, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or a combination of two or more kinds thereof may be used. Further, an organic solvent other than the above-described solvents and water may be mixed and used. However, in consideration of the development characteristics, the amount of water to be blended into the rinse solution is preferably 30% by mass or less, more preferably 10% by mass or less, still more preferably 5% by mass or less, and particularly preferably 3% by mass or less with respect to the total amount of the rinse solution.

A known additive can be blended into the rinse solution as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse solution.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the method for forming a resist pattern of the present embodiment described above, since the above-described resist composition is used, a resist pattern in which high sensitivity can be achieved, lithography characteristics such as LWR are satisfactory, and the rectangularity is high can be formed.

### (Compound According to Third Aspect of Present Invention)

A compound (hereinafter, also referred to as “compound (B0)”) according to a third aspect of the present invention is represented by General Formula (b0).

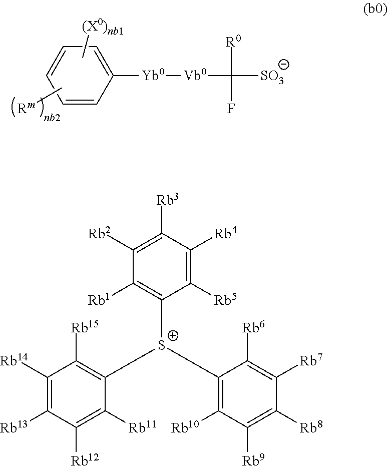

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, Yb⁰ represents a divalent linking group or a single bond, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group.]

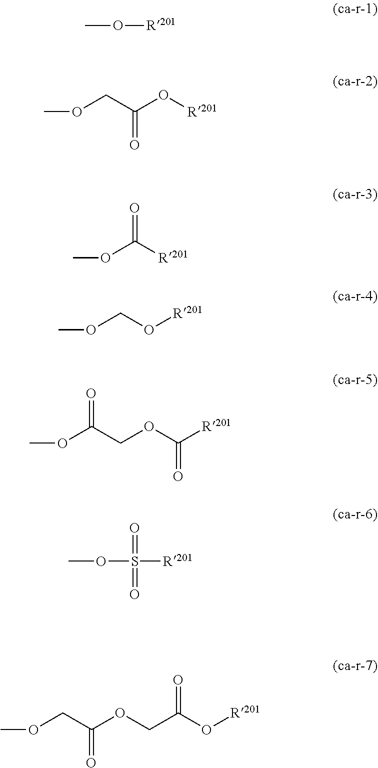

[In the formulae, R′²⁰¹'s each independently represent a hydrogen atom, a cyclic group which may have a substituent, a chain-like alkyl group which may have a substituent, or a chain-like alkenyl group which may have a substituent.]

The compound (B0) is the same compound as the component (B0) in the description of the resist composition according to the embodiment described above.

### (Method of Producing Compound (B0))

The compound (B0) can be obtained, for example, by a method including a step of performing a condensation reaction on a compound represented by General Formula (C-1) and a compound represented by General Formula (C-2) to obtain a compound (B0p) represented by General Formula (b0-p) (hereinafter, also referred to as “step A”) and a step of performing an ion exchange reaction on the compound (B0p) and a compound represented by General Formula (C′-3) to obtain a compound (D0) 5 represented by General Formula (b01-1) (hereinafter, also referred to as “step B′”).

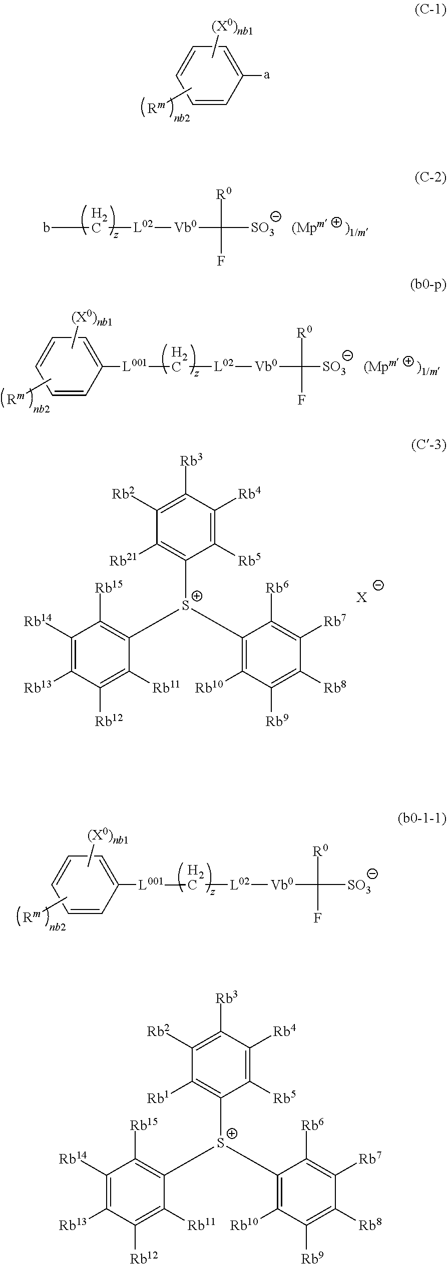

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, one of a and b represents a hydroxy group, and the other represents a carboxy group, z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater, L⁰⁰¹ represents an ester bond [—C(═O)—O— or —O—C(═O)—], X⁻ represents a counter anion, Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7). Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group.]

### <Step A>

The step A is a step of performing a condensation reaction on a compound represented by General Formula (C-1) (hereinafter, also referred to as “compound (C1)”) and a compound represented by General Formula (C-2) (hereinafter, also referred to as “compound (C2)”) to obtain a compound (B0p) represented by General Formula (b0-p) (hereinafter, also referred to as “compound (B0p)”).

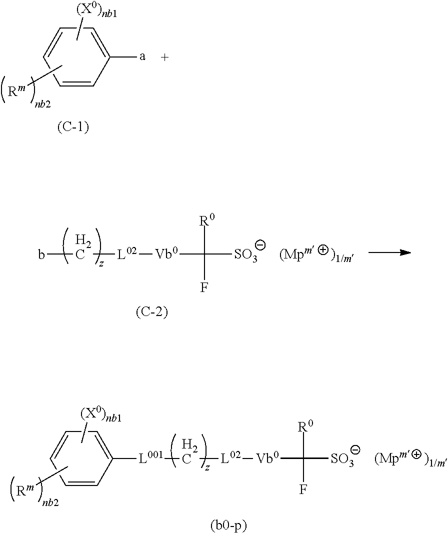

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, one of a and b represents a hydroxy group, and the other represents a carboxy group, z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a}), —C(R^{a})(N(R^{a})(R^{a}))—, or —C(═O)—N(R′)—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater, L⁰⁰¹ represents an ester bond [—C(═O)—O— or —O—C(═O)—].]

### <<Compound (C1)>>

The compound (C1) is a compound represented by General Formula (C-1).

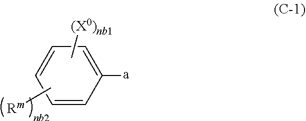

[In Formula (C-1), X⁰ represents a bromine atom or an iodine atom. R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, a represents a hydroxy group or a carboxy group.]

In General Formula (C-1), X⁰ represents a bromine atom or an iodine atom and preferably an iodine atom.

In General Formula (C-1), R^{m} represents a hydroxy group, an alkyl group, a fluorine atom, or a chlorine atom. The alkyl group as R^{m} is preferably an alkyl group having 1 to 5 carbon atoms and more preferably a methyl group or an ethyl group.

In General Formula (C-1), among the examples, it is preferable that R^{m} represents a hydroxy group or a fluorine atom.

In General Formula (C-1), nb1 represents an integer of 1 to 5, nb2 represents an integer of 0 to 4, and 1≤nb1+nb2≤5 is satisfied.

It is preferable that nb1 represents an integer of 1 to 3. nb2 represents preferably an integer of 0 to 3 and more preferably 0 or 1.

In General Formula (C-1), a represents a hydroxy group or a carboxy group, and one of a and b described below represents a hydroxy group and the other represents a carboxy group.

Specific examples of the compound (C1) in the method for producing a compound according to the present embodiment are shown below.

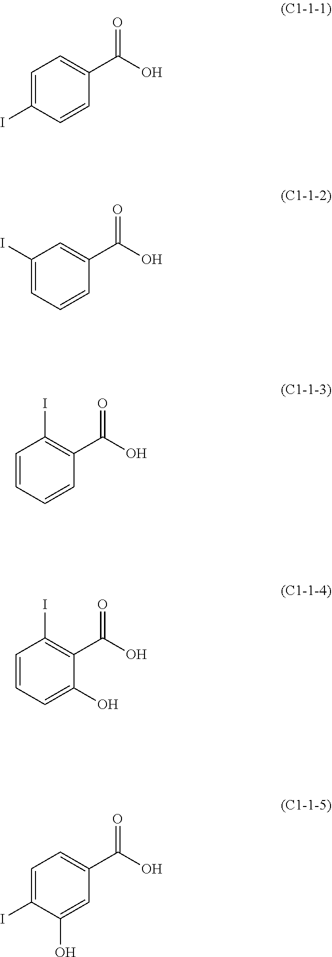 [SMILES: O=C(O)c1ccc(I)cc1 ; O=C(O)c1cccc(I)c1 ; O=C(O)c1ccccc1I ; O=C(O)c1c(O)cccc1I ; O=C(O)c1ccc(I)c(O)c1]

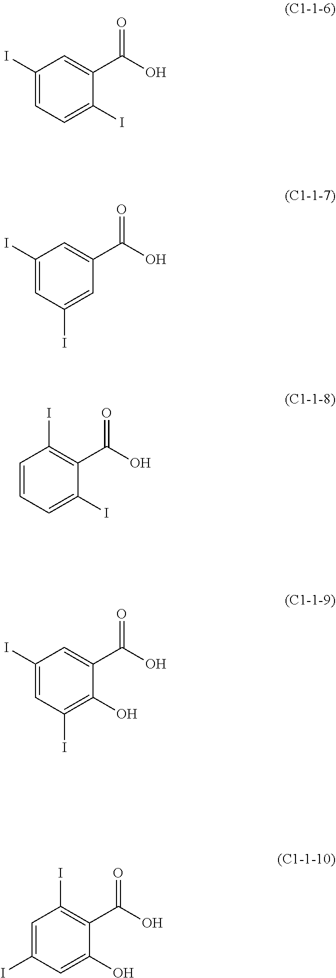 [SMILES: O=C(O)c1cc(I)ccc1I ; O=C(O)c1cc(I)cc(I)c1 ; O=C(O)c1c(I)cccc1I ; O=C(O)c1cc(I)cc(I)c1O ; O=C(O)c1c(O)cc(I)cc1I]

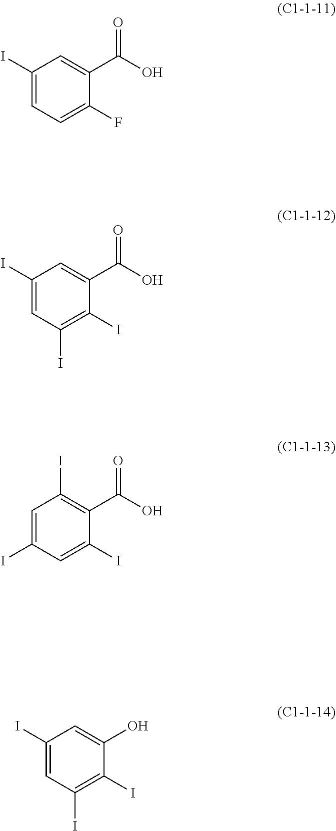 [SMILES: O=C(O)c1cc(I)ccc1F ; O=C(O)c1cc(I)cc(I)c1I ; O=C(O)c1c(I)cc(I)cc1I ; Oc1cc(I)cc(I)c1I]

<<Compound (C2)>>

The compound (C2) is a compound represented by General Formula (C-2).

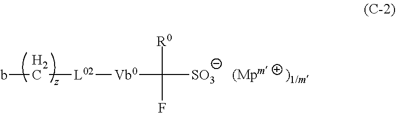

[In Formula (C-2), b represents a hydroxy group or a carboxy group. z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater.]

In General Formula (C-2), b represents a hydroxy group or a carboxy group, and one of a and b represents a hydroxy group and the other represents a carboxy group.

In General Formula (C-2), z represents an integer of 0 to 10, preferably an integer of 0 to 5, and more preferably an integer of 0 to 3.

In General Formula (C-2), L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—. R^{a}'s each independently represent a hydrogen atom or an alkyl group.

The alkylene group as L⁰² and the alkyl group as R^{a} each have preferably 1 to 4 carbon atoms and more preferably 1 to 3 carbon atoms.

In General Formula (C-2), among the examples, L⁰² represents preferably a single bond, —OCO—, or —COO—, more preferably a single bond or —COO—, and still more preferably a single bond.

In General Formula (C-2), Vb⁰ represents an alkylene group, a fluorinated alkylene group, or a single bond.

The alkylene group and the fluorinated alkylene group as Vb⁰ each have preferably 1 to 4 carbon atoms and more preferably 1 to 3 carbon atoms. Examples of the fluorinated alkylene group as Vb⁰ include a group obtained by substituting some or all hydrogen atoms in an alkylene group with a fluorine atom. Among these, Vb⁰ represents preferably an alkylene group having 1 to 4 carbon atoms, a fluorinated alkylene group having 1 to 4 carbon atoms, or a single bond and more preferably a group obtained by substituting some hydrogen atoms in an alkylene group having 1 to 3 carbon atoms with a fluorine atom, or a single bond.

In General Formula (C-2), R⁰ represents a hydrogen atom, a fluorinated alkyl group having 1 to 5 carbon atoms, or a fluorine atom. R⁰ represents preferably a fluorine atom or a perfluoroalkyl group having 1 to 5 carbon atoms and more preferably a fluorine atom.

In General Formula (C-2), Mp^{m′+} represents a metal cation or an organic ammonium cation having a Log P of 4.8 or less. m′ represents an integer of 1 or greater.

### Metal Cation

Examples of the metal cation include an alkali metal ion, an alkaline earth metal ion, a rubidium ion, a strontium ion, and a yttrium ion.

Among these, an alkali metal ion or an alkaline earth metal ion is preferable, an alkali metal ion is more preferable, a sodium ion or a lithium ion is still more preferable, and a sodium ion is particularly preferable.

Organic Ammonium Cation with Log P of 4.8 or Less

The organic ammonium cation is not particularly limited as long as the Log P is 4.8 or less. Further, the lower limit of the organic ammonium cation is not particularly limited, but is, for example, −1.0 or greater.

“Log P value” denotes the logarithmic value of the octanol/water partition coefficient (P_{ow}). “Log P value” is an effective parameter that can characterize the hydrophilicity/hydrophobicity of a wide range of compounds. Generally, the partition coefficient is determined by calculation regardless of an experiment, and in the present invention, the Log P value denotes a value calculated, for example, by CAChe Work System Pro Version 6.1. 12.33.

It means that the hydrophobicity increases in a case where the Log P value increases on a positive side greater than 0, and the water solubility increases (the polarity is high) in a case where the absolute value increases on a negative side. The Log P value has a negative correlation with the water solubility of an organic compound and is widely used as a parameter for estimating the hydrophilicity and hydrophobicity of an organic compound.

In a case where the cation moiety of the compound (C2) in the method for producing a compound according to the present embodiment is an organic ammonium cation and the Log P is 4.8 or less, the reaction in the step B′ described below proceeds smoothly, and the yield is improved. Further, a target compound can be obtained with less impurities.

Specific examples of the organic ammonium cation include a cation represented by General Formula (ca-p-1) and a cation represented by General Formula (ca-p-2).

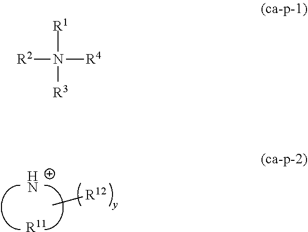

[In the formulae, R¹ to R⁴ each independently represent a hydrocarbon group which may have a substituent or a hydrogen atom. Here, at least one of R¹ to R⁴ represents a hydrocarbon group which may have a substituent. R¹¹ represents a group that forms an aromatic ring together with the nitrogen atom to which R¹¹ is bonded, R¹² represents an alkyl group or a halogen atom, and y represents an integer of 0 to 5.]

In General Formula (ca-p-1), R¹ to R⁴ each independently represent a hydrocarbon group which may have a substituent or a hydrogen atom. The hydrocarbon groups as R¹ to R⁴ are each independently preferably a hydrocarbon group having 1 to 15 carbon atoms and more preferably a hydrocarbon group having 1 to 10 carbon atoms. Further, the total number of carbon atoms in the hydrocarbon group as R¹ to R⁴ is preferably in a range of 1 to 20, more preferably in a range of 3 to 18, and still more preferably in a range of 4 to 15.

Examples of the hydrocarbon group include a linear or branched alkyl group, a cyclic hydrocarbon group, or the like.

The linear or branched alkyl group is preferably a linear or branched alkyl group having 1 to 10 carbon atoms and more preferably a linear or branched alkyl group having 1 to 10 carbon atoms.

The cyclic hydrocarbon group may be an aliphatic hydrocarbon group or an aromatic hydrocarbon group.

As the cyclic aliphatic hydrocarbon group, a group in which one hydrogen atom has been removed from a monocycloalkane is preferable. The monocycloalkane has preferably 3 to 6 carbon atoms, and specific examples thereof include cyclopentane and cyclohexane.

As the cyclic aromatic hydrocarbon group, a phenyl group is preferable.

Examples of the substituent which may be included in the hydrocarbon group as R¹ to R⁴ include an alkyl group, an alkoxy group, a hydroxyl group, an oxo group (═O), and an amino group.

In General Formula (ca-p-2), R¹¹ represents a group that forms an aromatic ring together with the nitrogen atom to which the R¹¹ is bonded. The aromatic ring is preferably a 4- to 7-membered ring, more preferably a 4- to 6-membered ring, and still more preferably a 6-membered ring.

In General Formula (ca-p-2), R¹² represents an alkyl group, and examples thereof include the same groups as those for the linear or branched alkyl group as R¹ to R⁴.

In General Formula (ca-p-2), y represents an integer of 0 to 5, preferably 1 or 0, and more preferably 0.

Specific examples of the cation moiety of the compound (C2) and Log P values calculated by CAChe Work System Pro Version 6.1.12.33 are shown below.

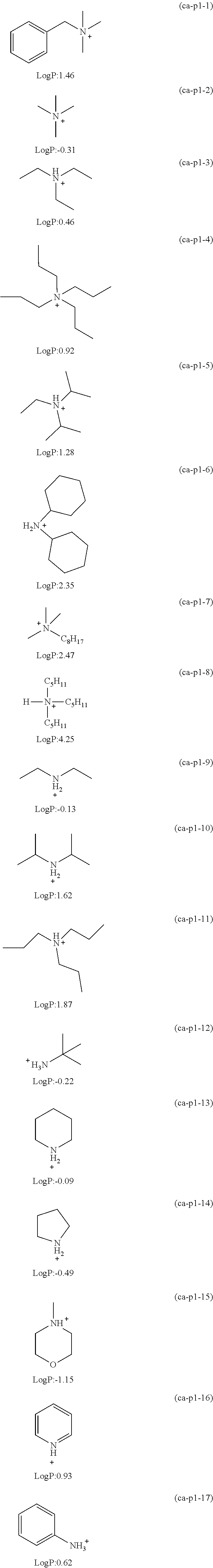

In General Formula (C-2), among the examples, Mp^{m′+} represents an organic ammonium cation having a Log P of 4.8 or less and more preferably a cation having a Log P of 4.8 or less and represented by General Formula (ca-p-1) or (ca-p-2) from the viewpoint that a target compound can be produced with less impurities and a higher yield.

Specific examples of the compound (C2) in the method for producing a compound according to the present embodiment are shown below.

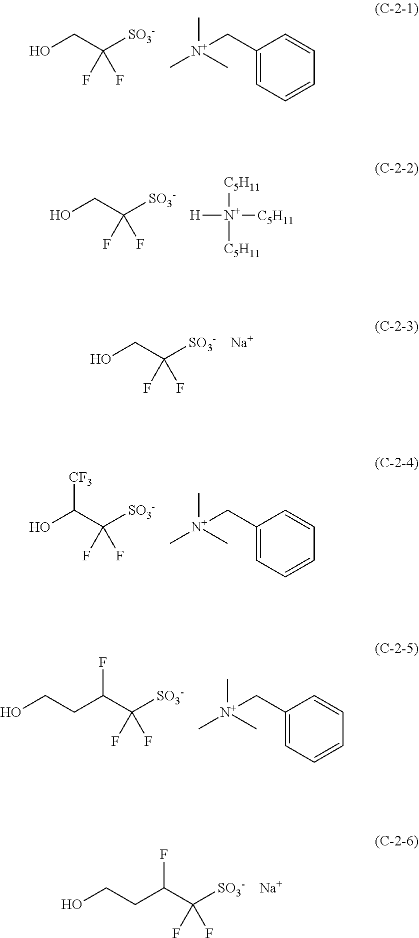

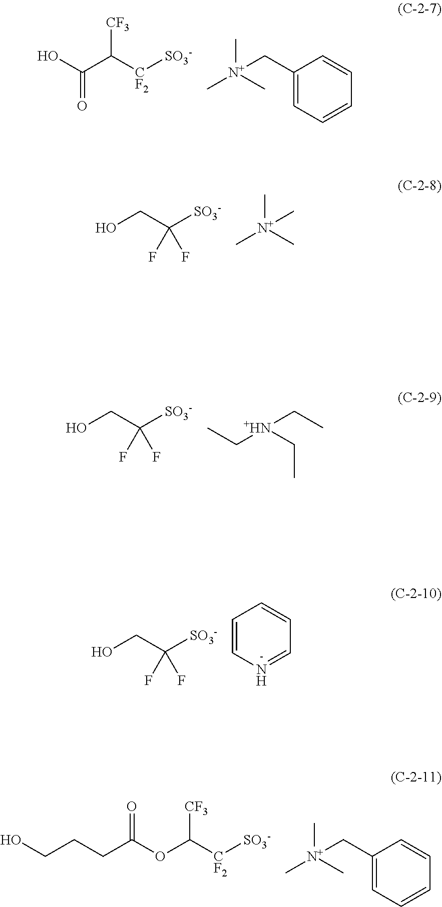

### <<Compound (B0p)>>

The compound (B0p) is a compound represented by General Formula (b0-p), which is obtained by performing a condensation reaction on the compound (C1) and the compound (C2) described above.

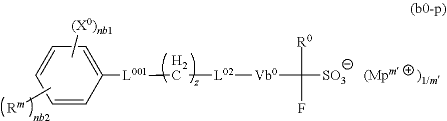

[In the formula, X⁰ represents a bromine atom or an iodine atom, L⁰⁰¹ represents an ester bond [—C(═O)—O— or —O—C(═O)—], R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a})—, or —C(═O)—N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, M^{m′+} represents a metal cation or an organic ammonium cation having a Log P of 4.8 or less, m′ represents an integer of 1 or greater.]

X⁰, R^{m}, nb1, and nb2 in General Formula (b0-p) each have the same definition as that for X⁰, R^{m}, nb1, and nb2 in General Formula (C-1).

z, L⁰², V^{h0}, R⁰, Mp^{m′+}, and m′ in General Formula (b0-p) each have the same definition as that for z, L⁰², V^{h0}, R⁰, Mp^{m′+}, and m′ in General Formula (C-2).

L⁰⁰¹ in General Formula (b0-p) represents an ester bond [—C(═O)—O— or —O— C(═O)-] that is formed by the condensation reaction between a in the compound (C1) and b in the compound (C2) described above.

Specific examples of the compound (B0p) in the method for producing a compound according to the present embodiment are shown below.

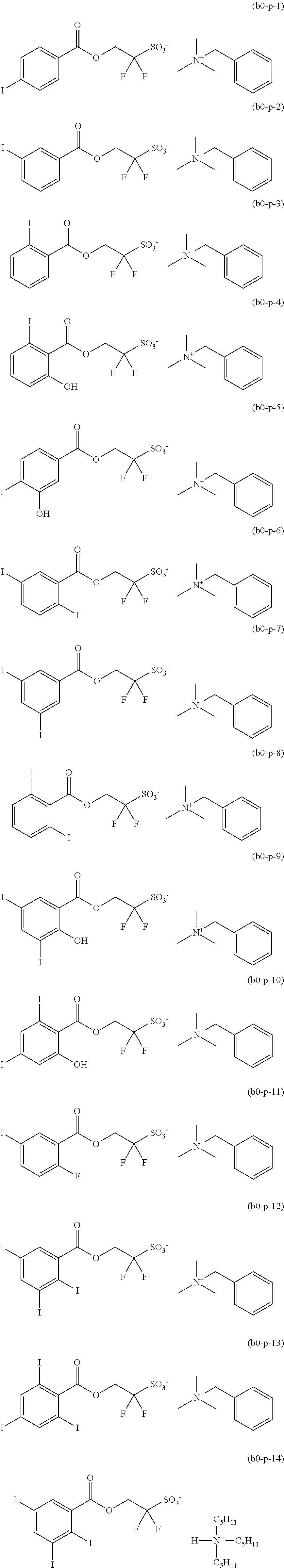

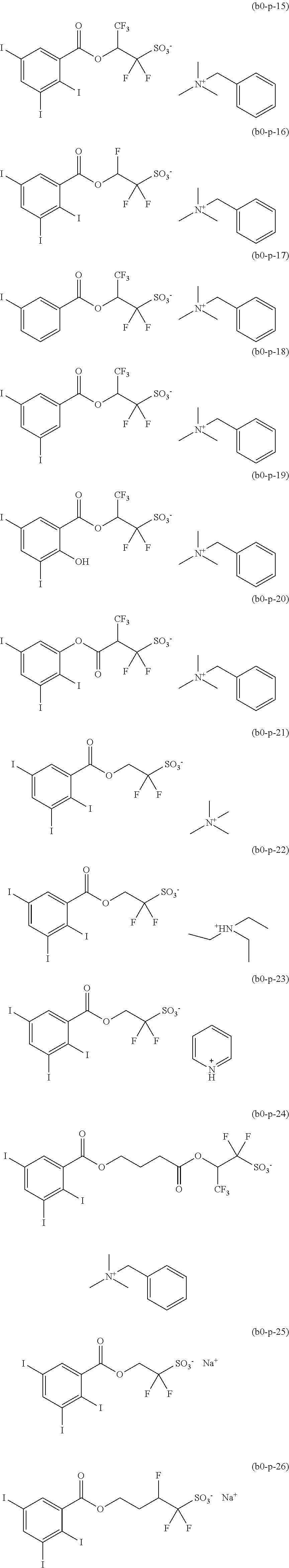

The condensation reaction in the step A may be carried out in the presence of a condensing agent and a base catalyst (additive).

Specific examples of the condensing agent include N,N′-dicyclohexylcarbodiimide, N,N′-diisopropylcarbodiimide (DIC), 1-ethyl-3-(3-dimethylaminopropyl)carbodiimide hydrochloride, and carbonyldiimidazole (CDI).

Specific examples of the base catalyst include tertiary amines such as trimethylamine, triethylamine, and tributylamine, aromatic amines such as pyridine, pyrrolidinopyridine, and 4-(dimethylamino)pyridine (DMAP), diazabicyclononene (DBN), and diazabicycloundecene (DBU).

In addition, the condensation reaction in the step A may be carried out in the presence of an acid catalyst.

Specific examples of the acid catalyst include diphosphorus pentoxide and methanesulfonic acid.

The reaction time of the step A is, for example, preferably 5 minutes or longer and 24 hours or shorter, more preferably in a range of 10 to 120 minutes, and still more preferably in a range of 10 to 60 minutes.

The reaction temperature of the step A is preferably in a range of 0° C. to 50° C. and more preferably in a range of 10° C. to 30° C.

Examples of the reaction solvent in the step A include dichloromethane, dichloroethane, chloroform, diethyl ether, tetrahydrofuran, N,N-dimethylformamide, acetonitrile, propionitrile, N,N′-dimethylacetamide, and dimethyl sulfoxide.

After the completion of the condensation reaction, the compound in the reaction solution may be isolated and purified. A known method in the related art can be used for isolation and purification, and for example, concentration, solvent extraction, distillation, crystallization, recrystallization, or chromatography can be appropriately combined and used.

### <Step B′>

The step B′ is a step of performing an ion exchange reaction on the compound (B0p) and a compound represented by General Formula (C′-3) to obtain a compound (B0) represented by General Formula (b01-1) (hereinafter, also referred to as “compound (B01-1)”).

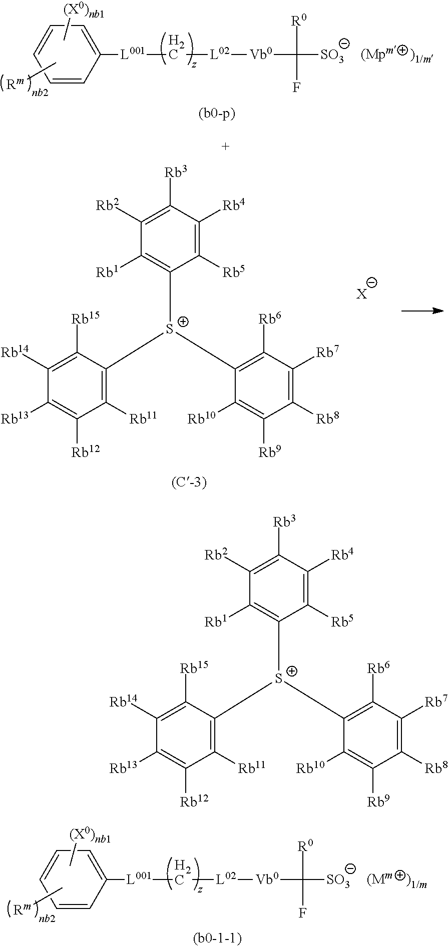

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰⁰1 represents an ester bond [—C(═O)—O— or —O—C(═O)—], z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a}) R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater, X⁻ represents a counter anion, Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7). Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group.]

### <<Compound (C′3)>>

The compound (C′3) is a compound represented by General Formula (C′-3).

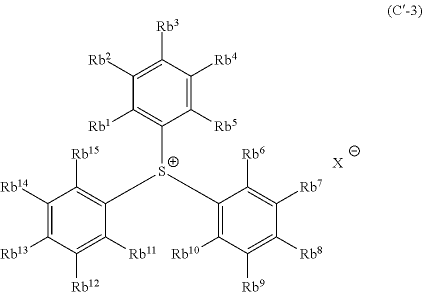

[In the formula, X⁻ represents a counter anion. Rb¹ to Rb¹⁵ each independently represent a hydrogen atom, an alkyl group, a halogen atom, a halogenated alkyl group, a carbonyl group, a cyano group, an amino group, an aryl group, or a group represented by any of General Formulae (ca-r-1) to (ca-r-7). Rb¹¹ and Rb¹² may be bonded to each other to form a ring with a sulfur atom in the formula, where at least two of Rb¹ to Rb⁵ represent a fluorine atom or at least one of Rb¹ to Rb⁵ represents a perfluoroalkyl group.]

In General Formula (C′-3), X⁻ represents a counter anion. Examples of X-include ions that can be an acid having a lower acidity than that of the compound (B0p), and specific examples thereof include a halogen ion such as a bromine ion or a chloride ion, BF₄⁻, AsF₆⁻, SbF₆⁻, PF₆⁻, and CO₄⁻.

In General Formula (C′-3), R^{b1} to Rb¹⁵ each have the same definition as that for R^{b1} to Rb¹⁵ in General Formula (b0).

### <<Compound (B01-1)>>

The compound (B0-1) is a compound represented by General Formula (b0-1-1), which is obtained by performing an ion exchange reaction on the compound (B0p) and the compound (C′3) described above.

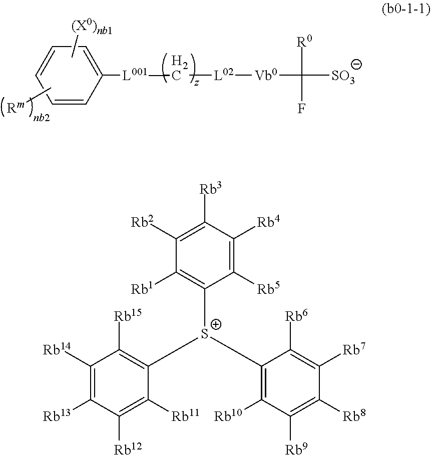

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰⁰¹ represents an ester bond [—C(═O)—O— or —O—C(═O)—], z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, M^{m+} represents an m-valent organic cation, and m represents an integer of 1 or greater.]

The anion moiety of the compound (B01-1) is the same as the anion moiety of the compound (B0p).

The cation moiety of the compound (B01-1) is the same as the cation moiety of the compound (C′3) described above.

The reaction time of the step B′ is, for example, preferably 0.5 minutes or longer and 24 hours or shorter, more preferably 5 minutes or longer and 12 hours or shorter, and still more preferably in a range of 10 to 60 minutes.

The reaction temperature of the step B′ is preferably in a range of 0° C. to 50° C. and more preferably in a range of 10° C. to 30° C.

As the reaction solvent in the step B′, for example, a mixed solvent of an organic solvent and water is preferable. Examples of the organic solvent include a ketone-based solvent such as cyclohexanone, methyl ethyl ketone, or diethyl ketone, an ether-based solvent such as diethyl ether, t-butyl methyl ether, or diisopropyl ether, a halogen-based solvent such as tetrahydrofuran, 1,3-dioxolane, dichloromethane, or 1,2-dichloroethane, an ester-based solvent such as ethyl acetate or propylene glycol monomethyl ether acetate, propionitrile, and a mixed solvent thereof.

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

As the raw material that is used in each step, a commercially available raw material may be used, or a synthetic material may be used.

### (Acid Generator According to Fourth Aspect of Present Invention)

The acid generator according to the fourth aspect of the present invention contains the compound according to the third aspect described above.

Such an acid generator is useful as an acid generator component for a chemically amplified resist composition. In a case where such an acid generator component is used in a chemically amplified resist composition, lithography characteristics such as reduction in roughness are improved, the pattern shape is satisfactorily maintained, and high sensitivity can be achieved. In a case where such an acid generator component is used, particularly, high sensitivity to an EB or EUV light source is easily obtained. In addition, according to the chemically amplified resist composition containing such an acid generator component, the resolution performance is further improved.

### (Method of Producing Compound According to Fifth Aspect of Present Invention)

A method for producing a compound according to a fifth aspect of the present invention includes a step of performing a condensation reaction on a compound represented by General Formula (C-1) and a compound represented by General Formula (C-2) to obtain a compound (B0p) represented by General Formula (b0-p) (hereinafter, also referred to as “step A”) and a step of performing an ion exchange reaction on the compound (B0p) and a compound represented by General Formula (C-3) to obtain a compound (b0′) represented by General Formula (b0′) (hereinafter, also referred to as “step B”).

The compound produced by the method for producing a compound according to the present embodiment is a compound useful as an acid generator for a resist composition. Specifically, the compound has an iodine atom having a high absorption cross-sectional area in the anion moiety with respect to EUV and EB. Accordingly, the sensitivity to EUV and EB can be further improved as compared with an acid generator having no iodine atom in the related art. In addition, since the component has an iodine atom in the anion moiety, the solubility in a developing solution can also be appropriately adjusted.

Therefore, the lithography characteristics can be further improved by allowing the resist composition to contain the compound.

### <Step A>

The step A is a step of performing a condensation reaction on a compound represented by General Formula (C-1) (hereinafter, also referred to as “compound (C1)”) and a compound represented by General Formula (C-2) (hereinafter, also referred to as “compound (C2)”) to obtain a compound (B0p) represented by General Formula (b0-p) (hereinafter, also referred to as “compound (B0p)”).

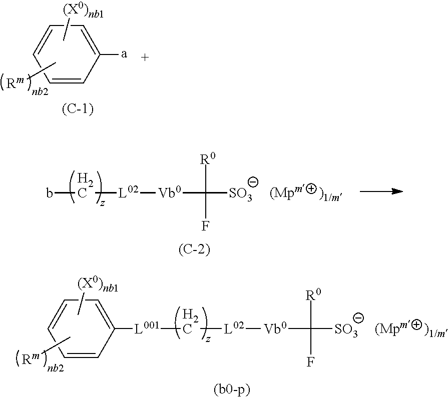

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, one of a and b represents a hydroxy group, and the other represents a carboxy group, z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater, L⁰⁰¹ represents an ester bond [—C(═O)—O— or —O—C(═O)—].]

The step (A) in the method for producing a compound according the present embodiment is the same as the above-described step (A).

### <Step B>

The step B is a step of performing an ion exchange reaction on the compound (B0p) and a compound represented by General Formula (C-3) to obtain a compound (b0′) represented by General Formula (b0′).

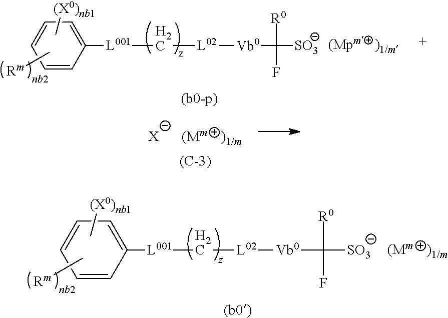

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰⁰¹ represents an ester bond [—C(═O)—O— or —O—C(═O)—], z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{d})(N(R^{a})(R^{a}))—, or —C(═O)—N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater, X⁻ represents a counter anion, M^{m+} represents an m-valent organic cation, and m represents an integer of 1 or greater.]

### <<Compound (C3)>>

The compound (C3) is a compound represented by General Formula (C-3).

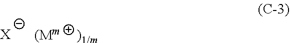

[In the formula, X⁻ represents a counter anion. M^{m+} represents an m-valent organic cation, and m represents an integer of 1 or greater.]

In General Formula (C-3), X⁻ represents a counter anion. Examples of X-include ions that can be an acid having a lower acidity than that of the compound (B0p), and specific examples thereof include a halogen ion such as a bromine ion or a chloride ion, BF₄⁻, AsF₆⁻, SbF₆⁻, PF₆⁻, and ClO₄⁻.

In General Formula (C-3), M^{m+} represents an m-valent organic cation. m represents an integer of 1 or greater. Among these, it is preferable that M^{m+} represents a sulfonium cation or an iodonium cation.

Preferred examples of the cation moiety ((M^{m+})_{t/m}) include organic cations each represented by General Formulae (ca-1) to (ca-5).

Specific examples of suitable cations represented by Formula (ca-1) include cations each represented by Chemical Formulae (ca-1-1) to (ca-1-72).

Among the examples, as the cation moiety ((M^{m+})_{t/m}), a cation represented by General Formula (ca-1) or (ca-2) is preferable.

<<Compound (b0′)>>

The compound (b0′) is a compound represented by General Formula (b0′), which is obtained by performing an ion exchange reaction on the compound (B0p) and the compound (C3) described above.

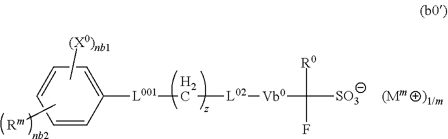

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰⁰¹ represents an ester bond [—C(═O)—O— or —O—C(═O)—], z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, M^{m+} represents an m-valent organic cation, and m represents an integer of 1 or greater.]

The anion moiety of the compound (b0′) is the same as the anion moiety of the compound (B0p).

The cation moiety of the compound (b0′) is the same as the cation moiety of the compound (C3) described above.

Specific examples of the compound (b0′) in the method for producing a compound according to the present embodiment are shown below.

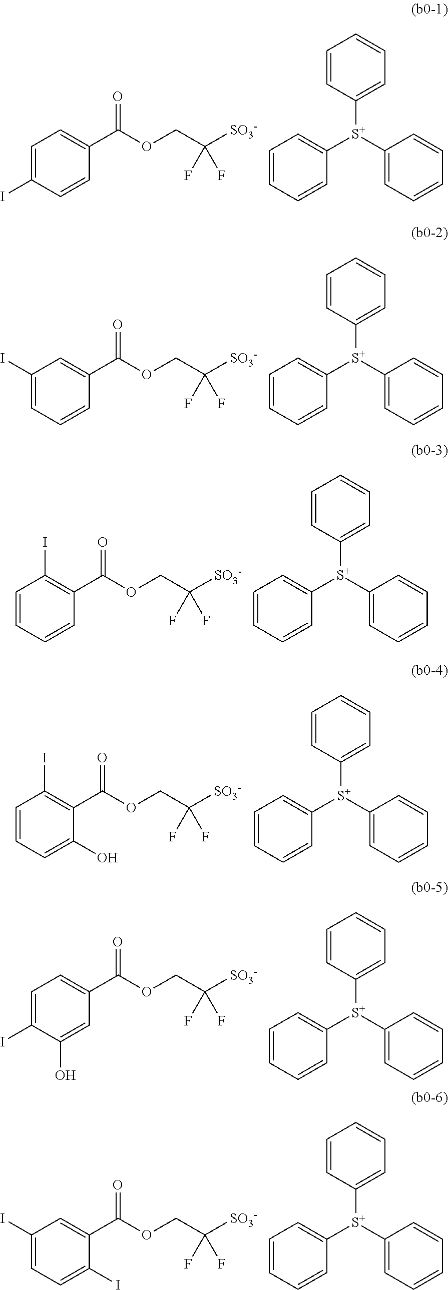

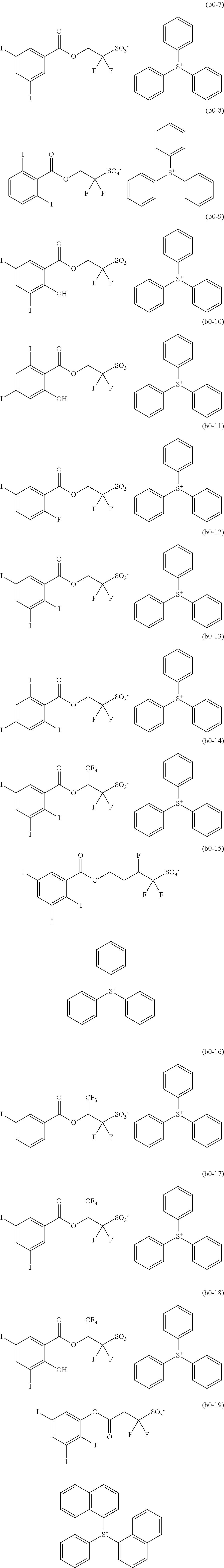

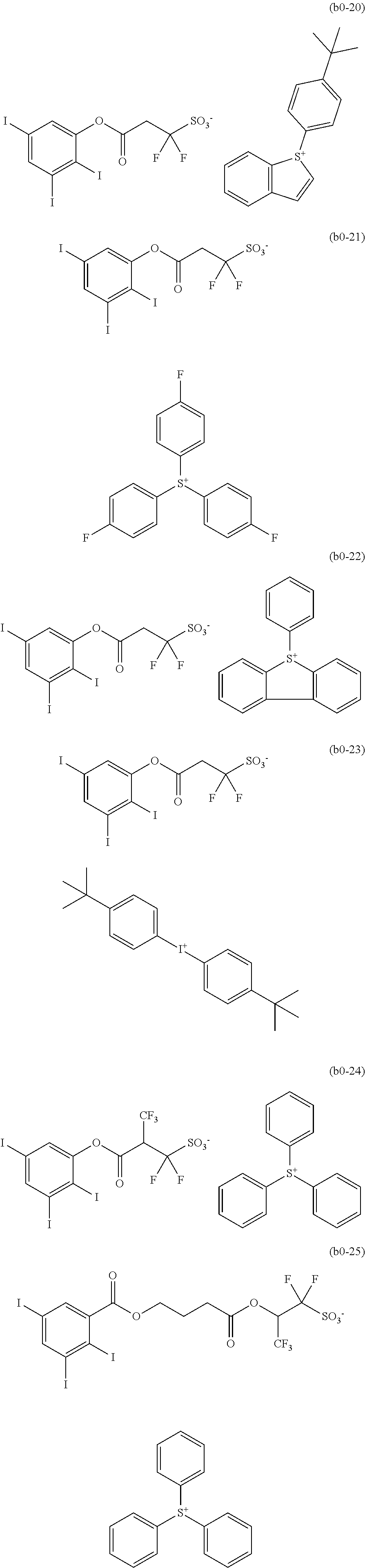

The reaction time of the step B is, for example, preferably 0.5 minutes or longer and 24 hours or shorter, more preferably 5 minutes or longer and 12 hours or shorter, and still more preferably in a range of 10 to 60 minutes.

The reaction temperature of the step B is preferably in a range of 0° C. to 50° C. and more preferably in a range of 10° C. to 30° C.

As the reaction solvent in the step B, for example, a mixed solvent of an organic solvent and water is preferable. Examples of the organic solvent include a ketone-based solvent such as cyclohexanone, methyl ethyl ketone, or diethyl ketone, an ether-based solvent such as diethyl ether, t-butyl methyl ether, or diisopropyl ether, a halogen-based solvent such as tetrahydrofuran, 1,3-dioxolane, dichloromethane, or 1,2-dichloroethane, an ester-based solvent such as ethyl acetate or propylene glycol monomethyl ether acetate, propionitrile, and a mixed solvent thereof.

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

As the raw material that is used in each step, a commercially available raw material may be used, or a synthetic material may be used.

Since the method for producing the compound according to the present embodiment described above employs a condensation reaction in the step A, the yield of the compound (B0p) can be improved as compared with a production method of the related art (for example, the SN2 reaction between a carboxylate anion and halogenated alkyl).

In addition, in the step B, since a metal cation or a relatively highly hydrophobic cation such as an organic ammonium cation having a Log P of 4.8 or less is employed as the cation moiety of the compound (C3) that reacts with the compound (Bop), the ion exchange reaction in the step B proceeds smoothly, and the yield of the compound (b0′) can be improved.

Therefore, according to the method for producing the compound of the present embodiment, a compound useful as an acid generator for a resist composition can be obtained with a high yield.

Further, in a case where a cation moiety having an organic ammonium cation with a Log P of 4.8 or less is employed as the cation moiety of the compound (C3) that reacts with the compound (B0p), a compound useful as an acid generator for a resist composition can be obtained with less impurities (for example, isomers and metals) and with a high yield.

### (Intermediate)

An intermediate according to the present embodiment is an intermediate used in the above-described method for producing a compound and represented by General Formula (b0-p).

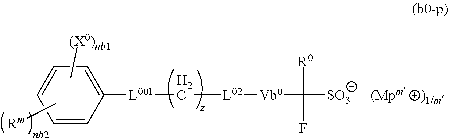

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1 S nb1±nb2 S 5 is satisfied, L⁰⁰¹ represents an ester bond [—C(═O)—O— or —O—C(═O)—], z represents an integer of 0 to 10. L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)N(R^{a})—, R^{a}'s each independently represent a hydrogen atom or an alkyl group. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater.]

The intermediate according to the present embodiment is the same as the compound (B0p) described above.

The intermediate according to the present embodiment is a compound produced in the middle of the above-described method for producing a compound, and the yield of the compound (b0′) can be improved by producing the compound (b0′) through the intermediate according to the present embodiment.

### (Compound)

The compound according to the present embodiment is a compound represented by General Formula (b0-p-1).

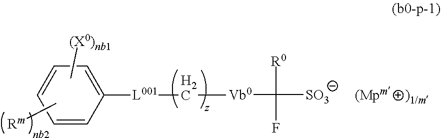

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, L⁰⁰¹ represents an ester bond [—C(═O)—O— or —O—C(═O)—], z represents an integer of 0 to 10. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater.]

The compound according to the present embodiment is a compound in which L⁰² of the compound (B0p) described above represents a single bond. The preferable aspects of the compound according to the present embodiment are the same as those of the compound (B0p) described above except that L⁰² of the compound (B0p) described above represents a single bond.

The compound according to the present embodiment is a compound produced in the middle of the above-described method for producing a compound, and the yield of the compound (b0′) can be improved by producing the compound (b0′) through the compound according to the present embodiment.

As the method for producing the compound according to the present embodiment, a compound represented by General Formula (C-1) and a compound represented by General Formula (C′-2) can be obtained by performing a condensation reaction.

The method for producing a compound according to the present embodiment is the same as the step A described above.

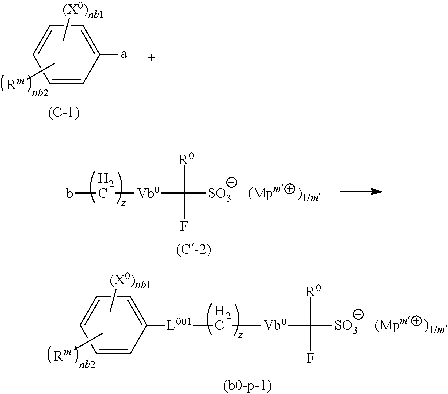

[In the formula, X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, where 1≤nb1+nb2≤5 is satisfied, one of a and b represents a hydroxy group, and the other represents a carboxy group, L⁰⁰¹ represents an ester bond [—C(═O)—O— or —O—C(═O)—], z represents an integer of 0 to 10. Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater.]

### EXAMPLES

Hereinafter, the present invention will be described in more detail based on examples, but the present invention is not limited to these examples.

### <Production of Polymer Compound>

Each of polymer compounds (A1-1) to (A1-4) was obtained by carrying out radical polymerization using monomers from which constitutional units constituting each of the polymer compounds were derived, at a predetermined molar ratio and performing a deprotection reaction.

The weight-average molecular weight (Mw) and the molecular weight dispersity (Mw/Mn) of each of the obtained polymer compounds were determined by GPC measurement (in terms of the standard polystyrene).

In addition, the copolymerization composition ratio (the proportion (molar ratio) of each constitutional unit in the structural formula) of each of the obtained polymer compounds was determined by the carbon 13 nuclear magnetic resonance spectrum (600 MHz_¹³C-NMR).
Polymer compound (A1-1): weight-average molecular weight (Mw) of 7,100, molecular weight dispersity (Mw/Mn) of 1.69, 1/m/n=40/50/10
Polymer compound (A1-2): weight-average molecular weight (Mw) of 7,000, molecular weight dispersity (Mw/Mn) of 1.72, 1/m/n=40/50/10
Polymer compound (A1-3): weight-average molecular weight (Mw) of 6900, molecular weight dispersity (Mw/Mn) of 1.72, 1/m/n=30/50/20
Polymer compound (A1-4): weight-average molecular weight (Mw) of 7,000, molecular weight dispersity (Mw/Mn) of 1.71, 1/m/n=40/50/10

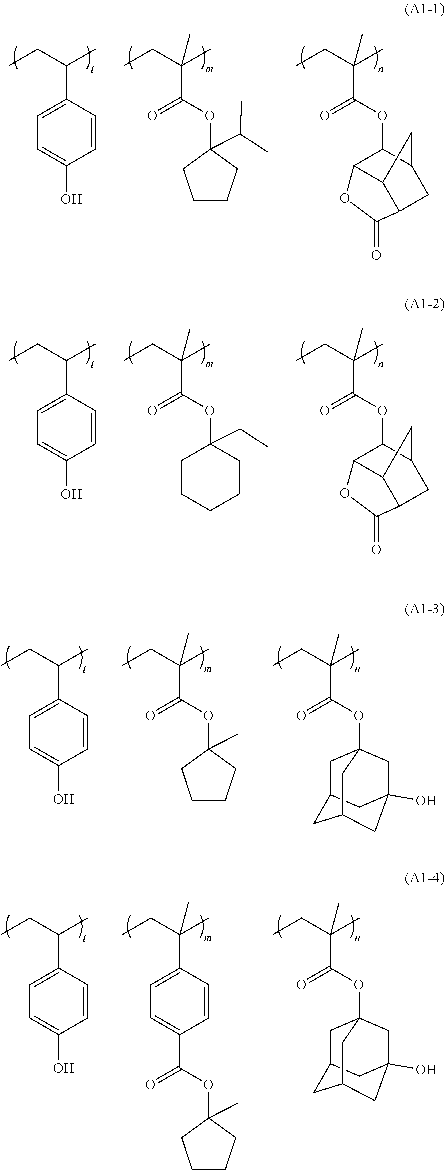

### <Synthesis of Compound (B0)>

### (Synthesis Example 1: Synthesis of Compound (B0-1))

Magnesium (8.3 g) and tetrahydrofuran (38 g) were stirred at 50° C., and a solution of the compound (Ca-N-1) (76 g) in tetrahydrofuran (150 g) was added dropwise thereto at the same temperature. After the completion of dropwise addition, the mixture was stirred for 2 hours and cooled to room temperature, and tetrahydrofuran (150 g) was added thereto, thereby obtaining a solution (1). The compound (Ca-E-1) (23 g) and tetrahydrofuran (150 g) were put into another container and stirred at room temperature. Trimethylsilyl trifluoromethanesulfonate (125 g) and the above-described solution (1) were added dropwise thereto. After the completion of dropwise addition, the reaction was continued at room temperature for 1 hour, and the reaction was completed. Thereafter, dichloromethane (200 g) was added thereto, the solution was stirred for 30 minutes, and the aqueous layer was removed. The organic layer was washed with ultrapure water (200 g) three times, and concentrated under reduced pressure. The concentrated residues were crystallized with dichloromethane/tert-butyl methyl ether, thereby obtaining a compound (Ca-1) (40 g) as a white solid.

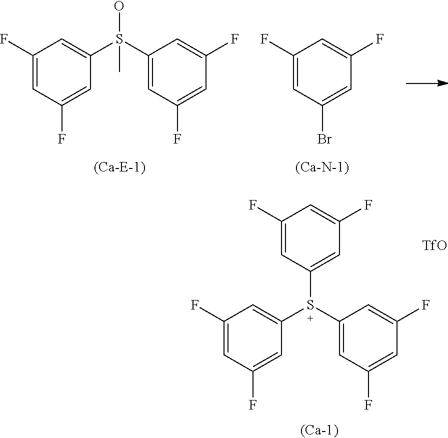

The compound (An-E-1) (4.0 g), N,N′-diisopropylcarbodiimide (1.06 g), 4-dimethylaminopyridine (0.1 g), the compound (An-N-1) (2.51 g), and dichloromethane (10 g) were put into a reaction container, and the mixture was stirred at room temperature. After confirmation of disappearance of the raw materials, the solid content was removed by filtration under reduced pressure. The filtrate was washed with ultrapure water (5.0 g) three times and concentrated under reduced pressure. The concentrated residue was crystallized with ethyl acetate/tert-butyl methyl ether, thereby obtaining a compound (An-1) (5.8 g).

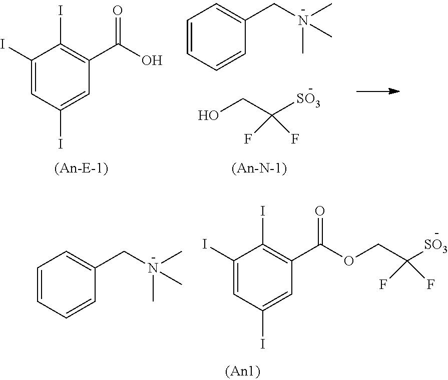

The compound (Ca-1) (4.0 g), the compound (An-1) (5.8 g), dichloromethane (20 g), and ultrapure water (20 g) were put into a reaction container and stirred at room temperature. The organic layer was washed with ultrapure water (10 g) five times and concentrated under reduced pressure, thereby obtaining a compound (B0-1) (7.4 g).

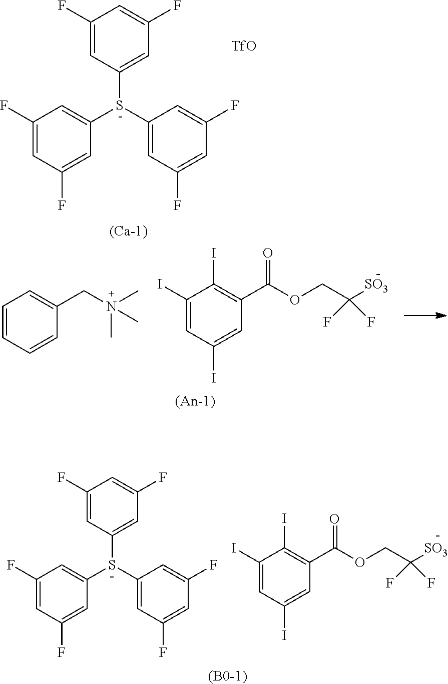

(Synthesis Examples 2 to 14: synthesis of compounds (B0-2) to (B0-14)) Compounds (B0-2) to (B0-14) were synthesized in the same manner as in Synthesis Example 1 except that the raw materials used were changed.

The obtained compounds (B0-1) to (B0-14) were subjected to NMR measurement, and the structures thereof were identified from the following analysis results.

### Compound (B0-1)

¹H-NMR (DMSO-d6, 400 MHz): δ (ppm)=8.11 (s, 1H), 8.00 (s, 1H), 6.85-6.65 (m, 9H), 5.13 (t, 2H)

¹⁹F-NMR (DMSO-d6, 376 MHz): δ (ppm)=−104.2, −114.3

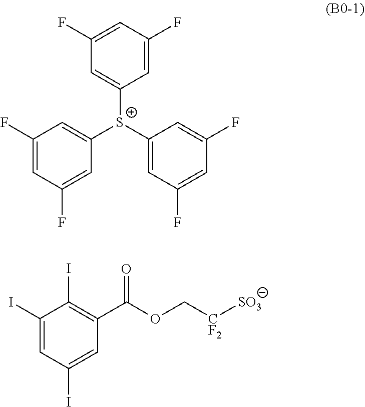

### Compound (B0-2)

¹H-NMR (DMSO-d6, 400 MHz): δ (ppm)=8.11 (s, 1H), 8.00 (s, 1H), 7.47 (d, 2H), 7.41-7.19 (m, 11H), 5.13 (t, 2H)

¹⁹F-NMR (DMSO-d6, 376 MHz): δ (ppm)=−56.9, −114.3

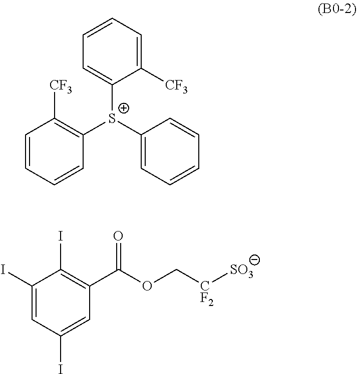

### Compound (B0-3)

¹H-NMR (DMSO-d6, 400 MHz): δ (ppm)=8.11 (s, 1H), 8.00 (s, 1H), 7.91 (s, 1H), 7.52 (s, 2H)), 7.39-7.30 (m, 10H), 5.13 (t, 2H)

¹⁹F-NMR (DMSO-d6, 376 MHz): δ (ppm)=−60.1, −114.3

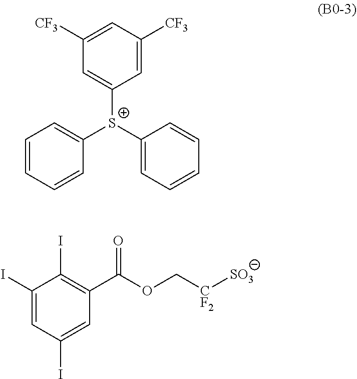

### Compound (B0-4)

¹H-NMR (DMSO-d6, 400 MHz): δ (ppm)=8.11 (s, 1H), 8.00 (s, 1H), 7.39-7.31 (m, 5H), 6.85-6.68 (m, 6H), 5.13 (t, 2H)

¹⁹F-NMR (DMSO-d6, 376 MHz): δ (ppm)=−103.9, −114.3

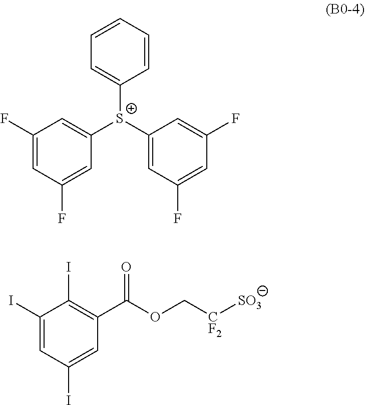

### Compound (B0-5)

¹H-NMR (DMSO-d6, 400 MHz): δ (ppm)=8.11 (s, 1H), 8.00 (s, 1H), 7.78-7.75 (m, 2H), 7.59-7.24 (m, 10H), 5.13 (t, 2H)

¹⁹F-NMR (DMSO-d6, 376 MHz): δ (ppm)=−61.9, −114.3

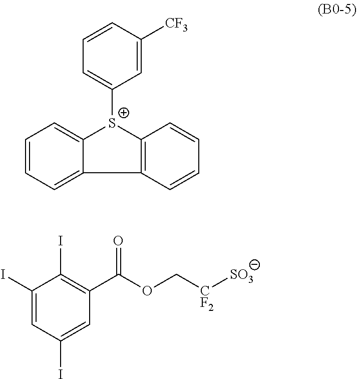

### Compound (B0-6)

¹H-NMR (DMSO-d6, 400 MHz): δ (ppm)=8.11 (s, 1H), 8.00 (s, 1H), 7.47 (d, 2H), 7.39-7.21 (m, 12H), 5.13 (t, 2H)

¹⁹F-NMR (DMSO-d6, 376 MHz): δ (ppm)=−58.1, −114.3

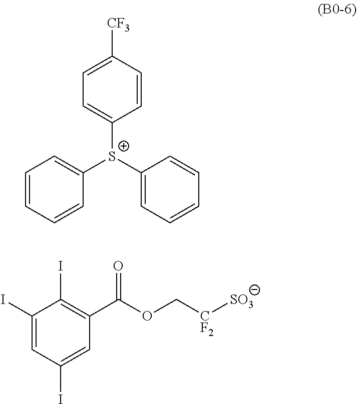

### Compound (B0-7)

¹H-NMR (DMSO-d6, 400 MHz): δ (ppm)=8.11 (s, 1H), 8.00 (s, 1H), 7.31 (d, 4H), 7.25-6.98 (m, 7H), 5.13 (t, 2H)

¹⁹F-NMR (DMSO-d6, 376 MHz): δ (ppm)=−105.2, −105.9, −113.5 to −114.3

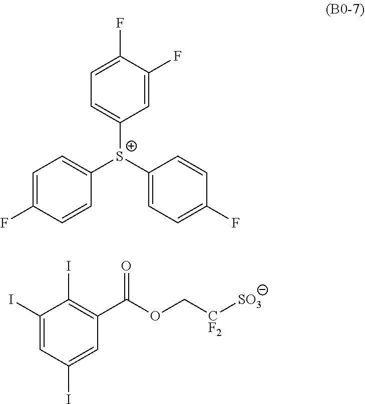

### Compound (B0-8)

¹H-NMR (DMSO-d6, 400 MHz): δ (ppm)=8.11 (s, 1H), 8.00 (s, 1H), 7.40-7.29 (m, 10H), 7.21-7.05 (m, 3H), 5.13 (t, 2H)

¹⁹F-NMR (DMSO-d6, 376 MHz): δ (ppm)=−105.9, −113.5 to −114.3

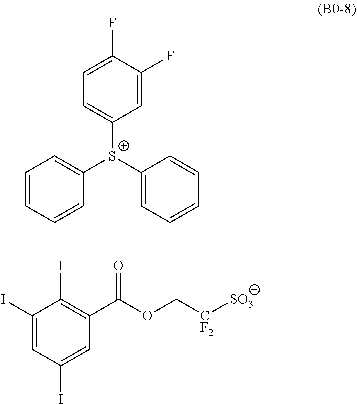

### Compound (B0-9)

¹H-NMR (DMSO-d6, 400 MHz): δ (ppm)=8.43-7.95 (m, 3H), 7.39-7.31 (m, 5H), 6.85-6.68 (m, 6H)

¹⁹F-NMR (DMSO-d6, 376 MHz): δ (ppm)=−72.4, −103.9, −113.0, −117.9

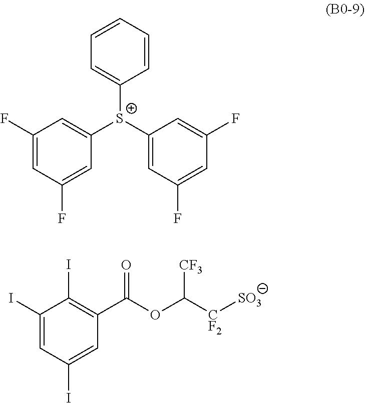

### Compound (B0-10)

¹H-NMR (DMSO-d6, 400 MHz): δ (ppm)=7.74 (s, 1H), 7.39-7.28 (m, 6H), 6.85-6.68 (m, 6H), 5.97-5.68 (m, 1H)

¹⁹F-NMR (DMSO-d6, 376 MHz): δ (ppm)=−72.4, −103.9, −113.5, −117.3

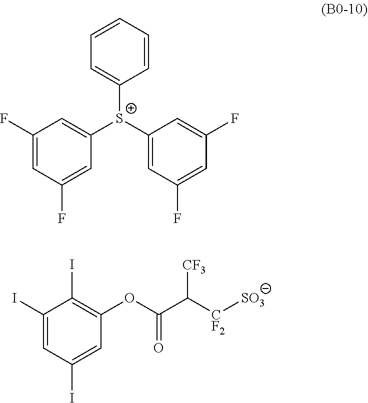

### Compound (B0-11)

¹H-NMR (DMSO-d6, 400 MHz): δ (ppm)=8.36 (s, 2H), 8.31-8.02 (m, 2H), 7.39-7.31 (m, 5H), 6.85-6.68 (m, 6H)

¹⁹F-NMR (DMSO-d6, 376 MHz): δ (ppm)=−72.4, −103.9, −113.1, −117.7

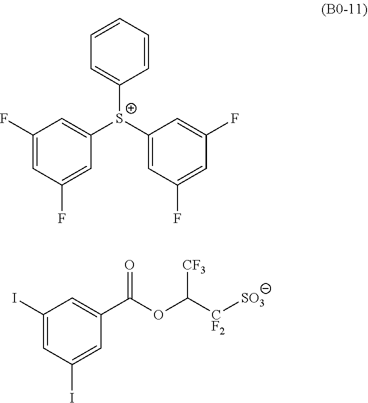

### Compound (B0-12)

¹H-NMR (DMSO-d6, 400 MHz): δ (ppm)=11.07 (s, 1H), 8.31-8.02 (m, 3H), 7.39-7.31 (m, 5H), 6.85-6.68 (m, 6H)

¹⁹F-NMR (DMSO-d6, 376 MHz): δ (ppm)=−72.4, −103.9, −115.2, −116.2

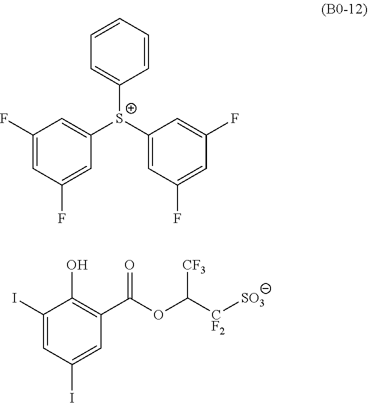

Compound (B0-13)

¹H-NMR (DMSO-d6, 400 MHz): δ (ppm)=8.37 (s, 1H), 8.31-7.89 (m, 3H), 7.39-7.19 (m, 6H), 6.85-6.68 (m, 6H)

¹⁹F-NMR (DMSO-d6, 376 MHz): δ (ppm)=−72.4, −103.9, −113.1, −117.7

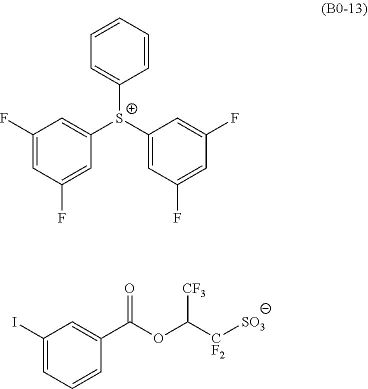

### Compound (B0-14)

¹H-NMR (DMSO-d6, 400 MHz): δ (ppm)=8.43-7.95 (m, 3H), 7.39-7.31 (m, 5H), 6.85-6.68 (m, 6H), 4.30 (t, 2H), 2.35 (t, 2H), 2.27-2.21 (m, 2H)

¹⁹F-NMR (DMSO-d6, 376 MHz): δ (ppm)=−72.4, −103.9, −113.5, −118.1

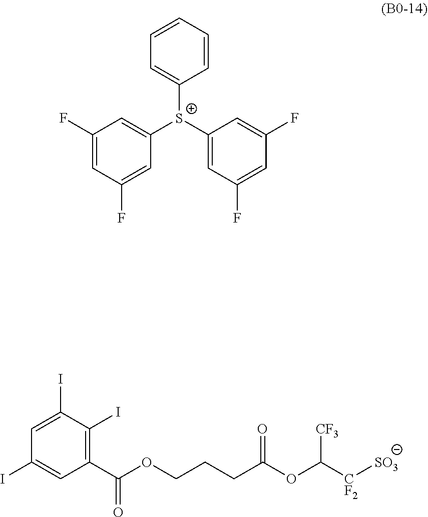

<Preparation of resist composition>

### Examples 1 to 17 and Comparative Examples 1 to 3

Each of the components listed in Tables 1 and 2 was mixed and dissolved to prepare a resist composition of each example.

TABLE 1

| | Component | Component | Component | Component |
|---|---|---|---|---|
| | (A) | (B) | (D) | (S) |
| Example 1 | (A)-1 | (B0)-1 | (D)-1 | (S)-1 |
| | [100] | [26.1] | [4.2] | [8000] |
| Example 2 | (A)-1 | (B0)-2 | (D)-1 | (S)-1 |
| | [100] | [26.8] | [4.2] | [8000] |
| Example 3 | (A)-1 | (B0)-3 | (D)-1 | (S)-1 |
| | [100] | [26.8] | [4.2] | [8000] |
| Example 4 | (A)-1 | (B0)-4 | (D)-1 | (S)-1 |
| | [100] | [25.2] | [4.2] | [8000] |
| Example 5 | (A)-1 | (B0)-5 | (D)-1 | (S)-1 |
| | [100] | [25] | [4.2] | [8000] |
| Example 6 | (A)-1 | (B0)-6 | (D)-1 | (S)-1 |
| | [100] | [25.1] | [4.2] | [8000] |
| Example 7 | (A)-1 | (B0)-7 | (D)-1 | (S)-1 |
| | [100] | [25.2] | [4.2] | [8000] |
| Example 8 | (A)-1 | (B0)-8 | (D)-1 | (S)-1 |
| | [100] | [24.2] | [4.2] | [8000] |
| Example 9 | (A)-1 | (B0)-9 | (D)-1 | (S)-1 |
| | [100] | [26.9] | [4.2] | [8000] |
| Example 10 | (A)-1 | (B0)-10 | (D)-1 | (S)-1 |
| | [100] | [26.9] | [4.2] | [8000] |
| Example 11 | (A)-1 | (B0)-11 | (D)-1 | (S)-1 |
| | [100] | [23.7] | [4.2] | [8000] |
| Example 12 | (A)-1 | (B0)-12 | (D)-1 | (S)-1 |
| | [100] | [24.1] | [4.2] | [8000] |
| Example 13 | (A)-1 | (B0)-13 | (D)-1 | (S)-1 |
| | [100] | [20.4] | [4.2] | [8000] |
| Example 14 | (A)-1 | (B0)-14 | (D)-1 | (S)-1 |
| | [100] | [29.1] | [4.2] | [8000] |
| Example 15 | (A)-2 | (B0)-4 | (D)-1 | (S)-1 |
| | [100] | [25.2] | [4.2] | [8000] |
| Example 16 | (A)-3 | (B0)-4 | (D)-1 | (S)-1 |
| | [100] | [25.2] | [4.2] | [8000] |
| Example 17 | (A)-4 | (B0)-4 | (D)-1 | (S)-1 |
| | [100] | [25.2] | [4.2] | [8000] |

TABLE 2

| | Component | Component | Component | Component |
|---|---|---|---|---|
| | (A) | (B) | (D) | (S) |
| Comparative | (A)-1 | (B1)-1 | (D)-1 | (S)-1 |
| Example 1 | [100] | [26] | [4.2] | [8000] |
| Comparative | (A)-1 | (B1)-2 | (D)-1 | (S)-1 |
| Example 2 | [100] | [25] | [4.2] | [8000] |
| Comparative | (A)-1 | (B1)-3 | (D)-1 | (S)-1 |
| Example 3 | [100] | [17.2] | [4.2] | [8000] |

In Tables 1 and 2, each abbreviation has the following meaning. The numerical values in the brackets are blending amounts (parts by mass).

(A1)-1 to (A1)-4: the polymer compounds (A1-1) to (A1-4).

(B0)-1 to (B0)-14: acid generators formed of compounds each represented by Chemical Formulae (B0-1) to (B0-14).

(B1)-1 to (B1)-3: acid generators formed of compounds each represented by Chemical Formulae (B1-1) to (B1-3).

(D)-1: an acid diffusion control agent formed of a compound represented by Chemical Formula (D-1).

(S)-1: A mixed solvent of propylene glycol monomethyl ether acetate/propylene glycol monomethyl ether=60/40 (in terms of mass ratio)

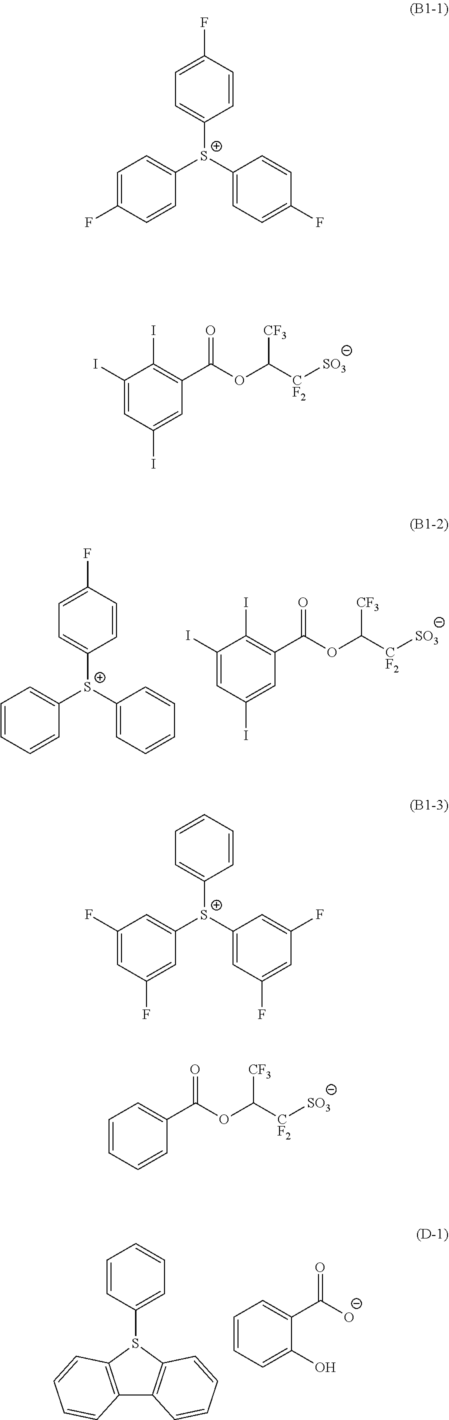

### <Formation of Resist Pattern>

A silicon substrate on which a hexamethyldisilazane (HMDS) had been performed was coated with the resist composition of each example using a spinner, and a prebake (PAB) treatment was performed thereon on a hot plate at a temperature of 110° C. for 60 seconds so that the composition was dried, thereby forming a resist film having a film thickness of 30 nm.

Next, drawing (exposure) was carried out on the resist film by using an electron beam lithography apparatus JEOL-JBX-9300FS (manufactured by JEOL Ltd.), with the target size set to a 1:1 line and space pattern (hereinafter, referred to as “LS pattern”) of a line width of 50 nm, at an acceleration voltage of 100 kV. Thereafter, a post exposure bake (PEB) treatment was performed thereon at 110° C. for 60 seconds.

Subsequently, alkali development was performed at 23° C. for 60 seconds using a 2.38 mass % tetramethylammonium hydroxide (TMAH) aqueous solution “NMD-3” (trade name, manufactured by TOKYO OHKA KOGYO CO., LTD.).

Thereafter, water rinsing was performed for 15 seconds using pure water.

As a result, a 1:1 LS pattern having a line width of 50 nm was formed.

### [Evaluation of Optimum Exposure Amount (Eop)]

According to <Formation of resist pattern>described above, an optimum exposure amount Eop (μC/cm²) for forming the LS pattern having the target size was determined. The results are listed in Tables 3 and 4 in the columns of “Eop (pC/cm²)”.

### [Evaluation of Line Width Roughness (LWR)]

Using the LS pattern formed in the section of “formation of resist pattern”, the 36 which is the scale that indicates the LWR was acquired. The results are listed in Table 3 and Table 4 in the colunms of “LWR (nm)”.

“3σ ” indicates a triple value (3σ) (unit: nm) of the standard deviation (σ) determined from measurement results obtained by measuring 400 line positions in the longitudinal direction of the line with a scanning electron microscope (acceleration voltage: 800 V, product name: S-9380, manufactured by Hitachi High-Tech Corporation). The results are shown in Table 3 and Table 4 as “LWR (nm)”.

In a case where the value of the 3a is small, this indicates that the roughness of a line side wall is small and an LS pattern with a uniform width is obtained.

### [Evaluation of Pattern Shape]

The cross-sectional shape of the LS pattern formed at the optimum exposure amount in the section of <Formation of resist pattern>described above was observed with an SEM (scanning electron microscope, acceleration voltage: 800 V, product name: SU-8000, manufactured by Hitachi High-Tech Corporation), a line width Lb in the middle of the resist pattern in the height direction and a line width La in an upper portion of the resist pattern were measured, the value of La/Lb was calculated, and this value was used as an index for evaluating the LS pattern shape. The rectangularity of the pattern was evaluated as satisfactory in a case where 0.9≤(La/Lb)≤1.1 was satisfied and evaluated as poor in a case otherwise. The results are listed in Tables 3 and 4 in the colunms of “pattern shape”.

TABLE 3

| | Eop (μC/cm²) | LWR (nm) | Pattern shape |
|---|---|---|---|
| Example 1 | 80 | 4.1 | 1.00 |
| Example 2 | 81 | 4.0 | 1.01 |
| Example 3 | 84 | 4.2 | 1.01 |
| Example 4 | 85 | 4.3 | 1.00 |
| Example 5 | 86 | 4.3 | 0.99 |
| Example 6 | 93 | 4.6 | 1.03 |
| Example 7 | 95 | 4.7 | 1.04 |
| Example 8 | 96 | 4.8 | 1.05 |
| Example 9 | 85 | 4.2 | 1.00 |
| Example 10 | 86 | 4.3 | 0.99 |
| Example 11 | 90 | 4.4 | 1.04 |
| Example 12 | 89 | 4.5 | 0.95 |
| Example 13 | 94 | 4.7 | 1.06 |
| Example 14 | 87 | 4.4 | 0.98 |
| Example 15 | 86 | 4.2 | 1.01 |
| Example 16 | 85 | 4.3 | 0.99 |
| Example 17 | 86 | 4.3 | 0.98 |

TABLE 4

| | Eop (μC/cm²) | LWR (nm) | Pattern shape |
|---|---|---|---|
| Comparative | 104 | 5.2 | 1.14 |
| Example 1 | | | |
| Comparative | 108 | 5.4 | 1.17 |
| Example 2 | | | |
| Comparative | 105 | 5.1 | 0.86 |
| Example 3 | | | |

As listed in Tables 3 and 4, it was confirmed that in the resist compositions of Examples 1 to 17, high sensitivity was achieved and the LWR and the pattern shape were excellent in the formation of the resist pattern.

In the present examples described below, a compound represented by Chemical Formula (C-1-1) is referred to as “compound (C-1-1)”, and the same applies to the compounds represented by other chemical formulae.

The compounds (C-1-1) to (C-1-14), the compounds (C-2-1) to (C-2-11), the compounds (X-1) to (X-3), and the compounds (C-3-1) to (C-3-6), used as the raw materials, are shown below.

Further, the combination of raw materials, the obtained intermediates (the compound (B0p) and the like), and the final target products (the compound (b0′) and the like) are listed in Tables 5 to 8.

Further, the compounds (C-2-1), (C-2-2), (C-2-4), (C-2-5), (C-2-7) to (C-2-11), and (X-1) will be shown with the Log P values of the cation moieties.

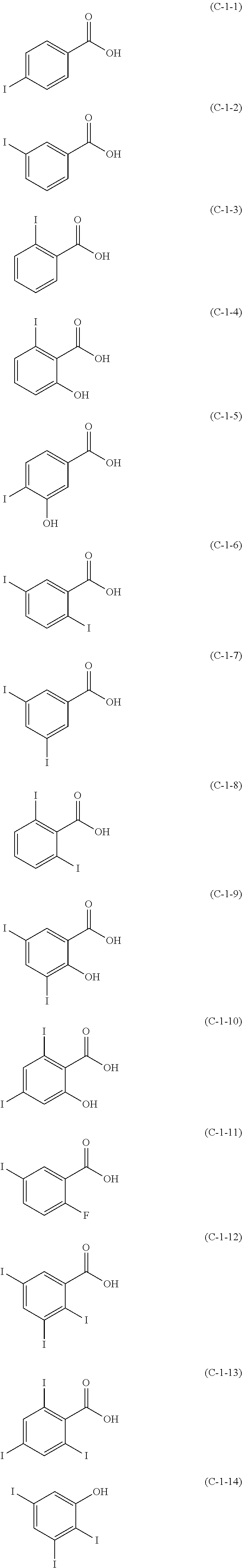

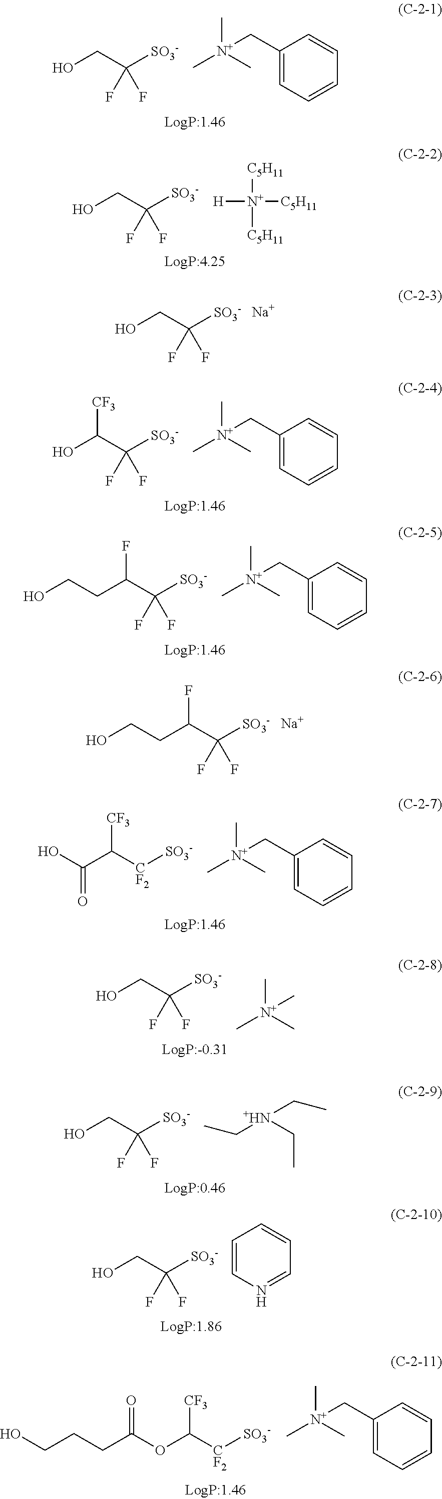

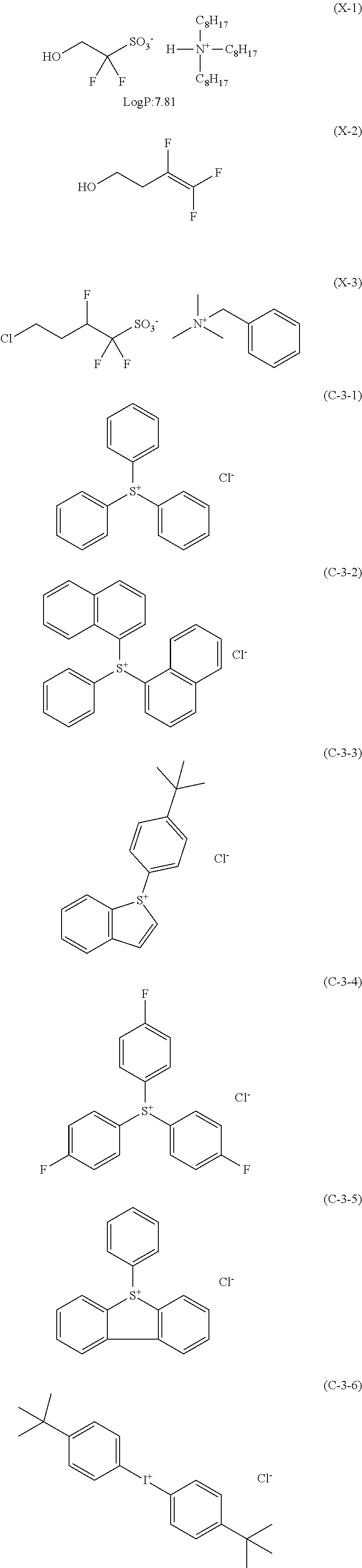

### <Step A: Step of Obtaining Compound (B0p)>

### Example 1a

2.00 g of the compound (C-1-1), 1.06 g of D-1, 0.1 g of E-1, 2.51 g of the compound (C-2-1), and 10.0 g of CH₂Cl₂ were put into a 100 mL eggplant flask and stirred at room temperature. After the confirmation of the disappearance of the raw materials, the solid content was removed by filtration under reduced pressure. The filtrate was transferred to a separatory funnel and washed with water three times, and the organic layer was concentrated under reduced pressure. The concentrated residues were heated and dissolved in 10 g of ethyl acetate and cooled, and 30 g of tBuOMe was added thereto to precipitate the solid content. The solid content was collected by filtration under reduced pressure and dried under reduced pressure, thereby obtaining 3.97 g of a compound (B0p-1).

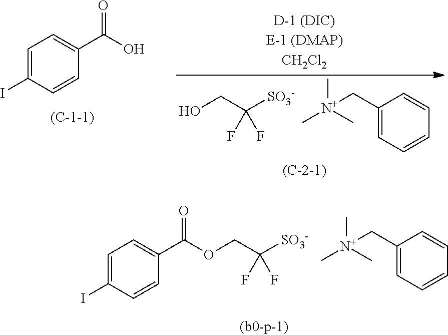

### (Examples 2a to 30a and 32a to 39a)

Each of compounds (B0p-2) to (B0p-19) and (B0p-21) to (B0p-26) was obtained by the same method as in the step of obtaining the compound (B0p-1) of Example 1a except that the used raw materials were changed to the compounds listed in Tables 1 to 3.

### Example 31a

1.80 g of diphosphorus pentoxide, 15 g of chloroform, and 0.47 g of diethyl ether were put into a 100 mL eggplant flask and stirred at room temperature for 1 hour. 3.00 g of the compound (C-1-14) and 2.6 g of the compound (C-2-7) were added thereto, and the mixture was stirred at room temperature for 24 hours. After the confirmation of the disappearance of the raw materials, the mixture was transferred to a separatory funnel and washed with water four times, and the organic layer was concentrated under reduced pressure. The concentrated residues were heated and dissolved in 10 g of ethyl acetate and cooled, and 30 g of tBuOMe was added thereto to precipitate the solid content. The solid content was collected by filtration under reduced pressure and dried under reduced pressure, thereby obtaining 4.58 g of a compound (B0p-20).

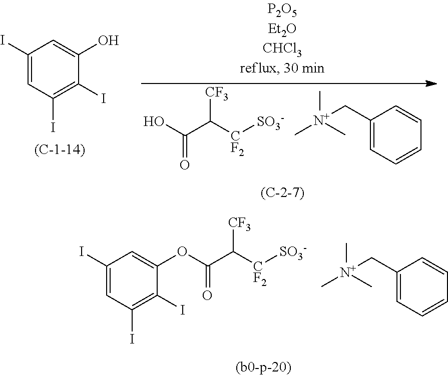

### (Comparative Example 1a)

22.00 g of the compound (C-1-12), 0.53 g of D-1, 0.05 g of E-1, 0.50 g of the compound (X-2), and 10.0 g of CH₂Cl₂ were put into a 100 mL eggplant flask and stirred at room temperature. After the confirmation of the disappearance of the raw materials, the solid content was removed by filtration under reduced pressure. The filtrate was transferred to a separatory funnel and washed with water three times, and the organic layer was concentrated under reduced pressure. The concentrated residues were heated and dissolved in ethyl acetate and cooled, and 30 g of tBuOMe was added thereto to precipitate the solid content. The solid content was collected by filtration under reduced pressure and dried under reduced pressure, thereby obtaining 1.96 g of a compound (b1-pre).

Next, 1.96 g of the compound (b1-pre), 0.44 g of sodium hydrogen sulfite, 0.53 g of sodium sulfite, and 10 g of water were added to a 100 mL three-necked flask, and the mixture was heated and refluxed for 40 hours. The mixture was cooled to room temperature and extracted 3 times with 10 g of CH₂Cl₂. The organic layer was concentrated, thereby obtaining 1.28 g of a compound (B0p-26).

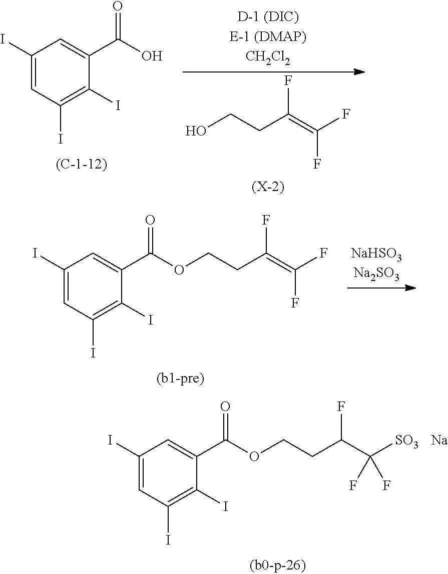

### Comparative Example 2a

22.00 g of the compound (C-1-12), 0.60 g of sodium iodide, 0.55 g of potassium carbonate, 1.50 g of the compound (X-3), and 10.0 g of dimethylformamide were put into a 100 mL eggplant flask and heated and stirred at an internal temperature of 90° C. for 24 hours. After the confirmation of the disappearance of the raw materials, the mixture was transferred to a separatory funnel, 30 g of CH₂C1₂ and 30 g of water were added thereto to separate the liquid from the mixture, and the organic layer was concentrated under reduced pressure. The concentrated residues were heated and dissolved in ethyl acetate and cooled, and 30 g of tBuOMe was added thereto to precipitate the solid content. The solid content was collected by filtration under reduced pressure and dried under reduced pressure, thereby obtaining 1.85 g of a compound (B0p-16).

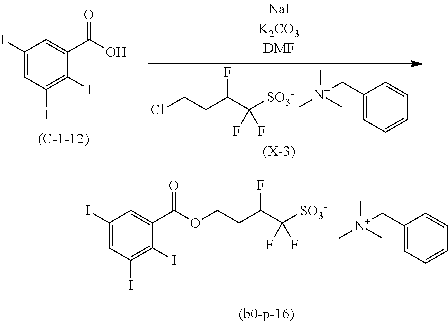

### Comparative Example 3a

A compound (B1p-1) was obtained by the same method as in the step of obtaining the compound (B0p-1) of Example 1 except that the compound (C-1-1) was changed to the compound (C-1-12) and the compound (C-2-1) was changed to the compound (X-1).

The obtained compounds (B0p-1) to (B0p-26) and (B1p-1) are each shown below.

In the present examples described below, a compound represented by Chemical Formula (b0-p-1) is referred to as “compound (b0p-1)”, and the same applies to the compounds represented by other chemical formulae.

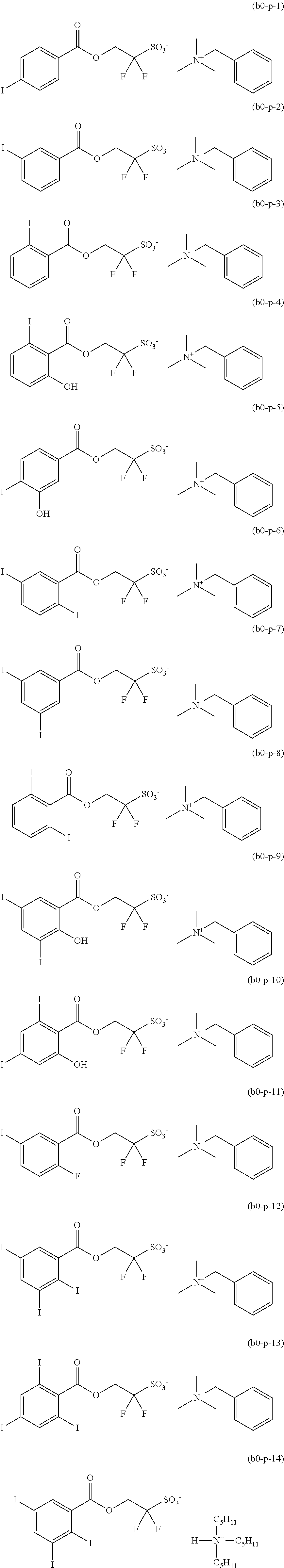

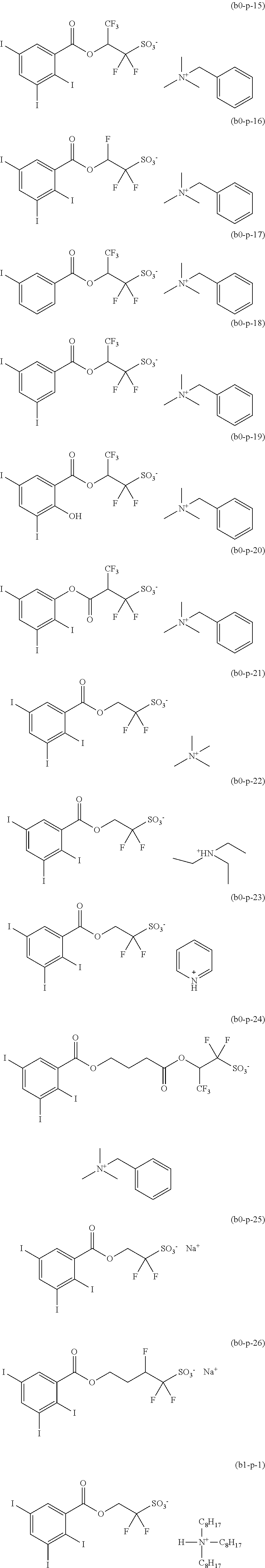

<Step B: Step of Obtaining Compound (b0′)>

### Example 1a

3.97 g of the compound (B0p-1), 2.19 g of the compound (C-3-1), 25 g of CH₂Cl₂, and 25 g of water were put into a 100 mL separatory funnel, and the liquid was separated from the mixture. The aqueous layer was removed, and the organic layer was washed with hydrochloric acid, washed with water, and concentrated under reduced pressure. The concentrated residues were dissolved in 5 g of CH₂C1₂ and stirred. 15 g of tBuOMe was added to this solution to precipitate crystals. The solid content was collected by filtration under reduced pressure and dried under reduced pressure, thereby obtaining 4.41 g of a compound (b0-1).

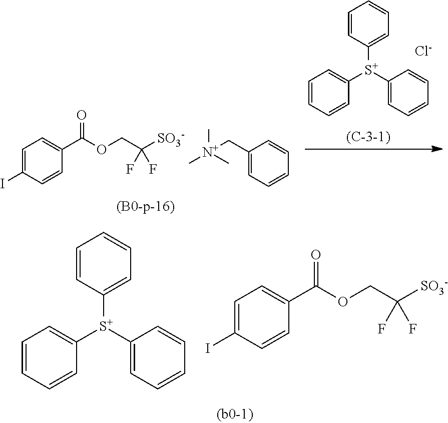

### Examples 2a to 36a and Comparative Examples 1a to 3a

Each of the following compounds (b0-2) to (b0-31) was obtained by the same method for producing the compound (b0-1) of Example 1a except that the combination of the compound (B0p-1) in the production of the compound (b0-1) of Example 1a and the salt exchange compound (C-3-1) was changed to the above-described compounds (B0p-1) to (B0p-26) and the salt exchange compounds (C-3-1) to (C-3-6).

Each of the obtained compounds (b0-1) to (b0-31) is shown below.

In the present examples, a compound represented by Chemical Formula (b0-1) is referred to as “compound (b01)”, and the same applies to the compounds represented by other chemical formulae.

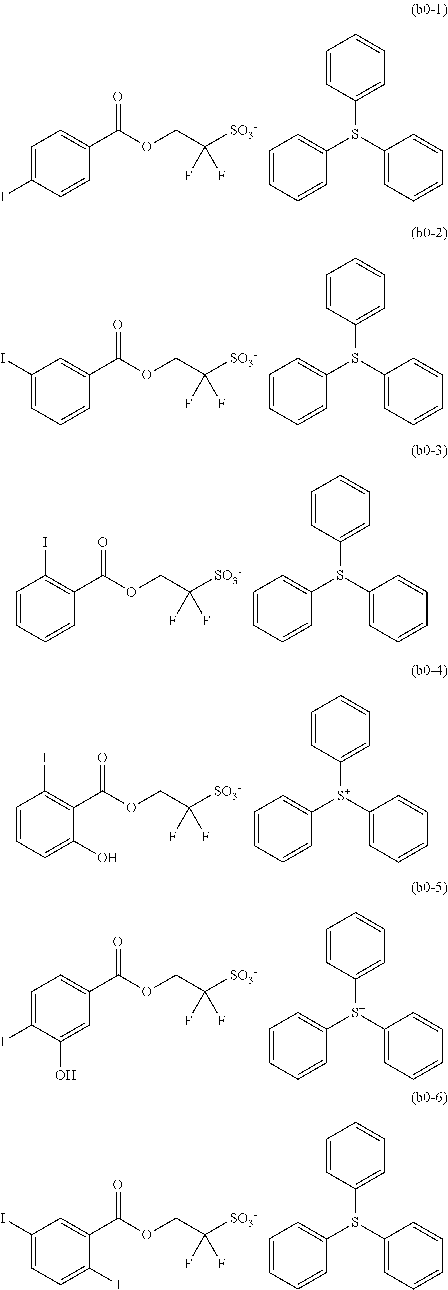

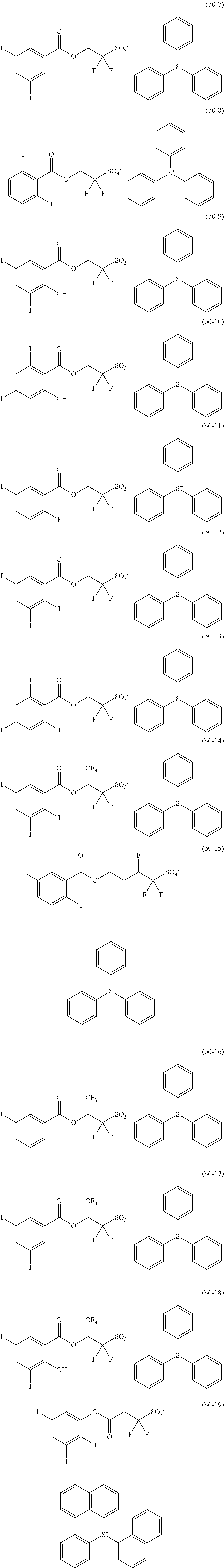

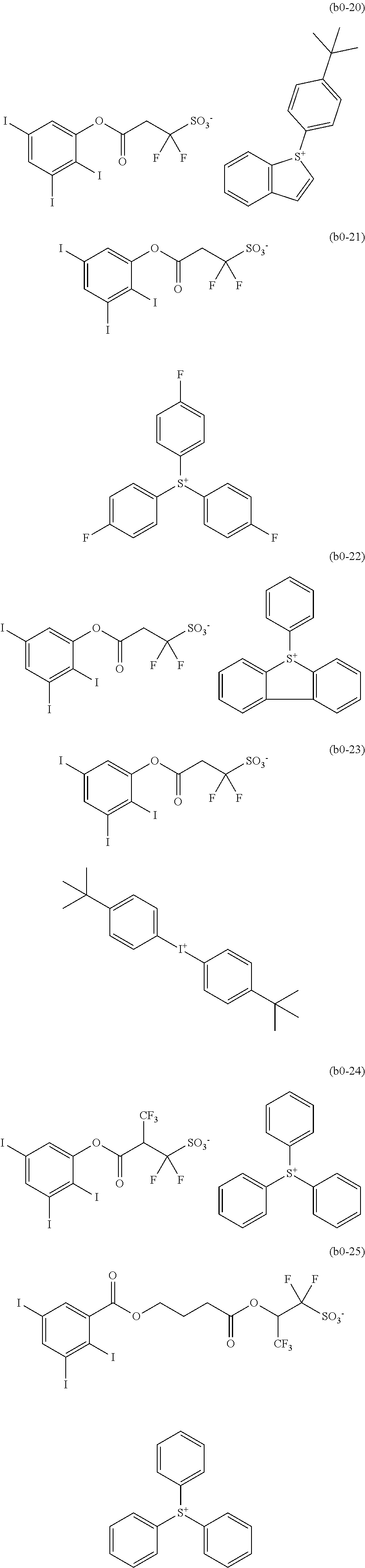

Further, the compound (C-2-11) used as a raw material for the compound (B0p-14) described above was synthesized by the following method.

2.00 g of butanoic acid. 4-2(tetrahydro-2H-pyran-2-yl)oxy]-, 1.2 of the compound D-1, 0.2 g of the compound E-1, 2.5 g of the compound (C-2-4), and 10.0 g of CH₂Cl₂ were put into a 100 mL eggplant flask and stirred at room temperature. After the confirmation of the disappearance of the raw materials, the solid content was removed by filtration under reduced pressure. The filtrate was transferred to a separatory funnel and washed with water three times, and the organic layer was concentrated under reduced pressure. The concentrated residues were heated and dissolved in 10 g of ethyl acetate and cooled, and 30 g of tBuOMe was added thereto to precipitate the solid content. The solid content was collected by filtration under reduced pressure and dried under reduced pressure, thereby obtaining 3.97 g of an intermediate.

This intermediate was put into a 100 mL eggplant flask, 1.0 g of TsOH and 10 g of dichloromethane were added thereto, and the mixture was stirred at room temperature. After the confirmation of the disappearance of the raw materials, 10 g of a 5% sodium hydrogen carbonate aqueous solution was added to stop the reaction, and the mixture was transferred to a separatory funnel. The aqueous layer was removed, and the organic layer was washed with water three times and concentrated. The concentrated residues were heated and dissolved in 10 g of ethyl acetate and cooled, and 30 g of tBuOMe was added thereto to precipitate the solid content. The solid content was collected by filtration under reduced pressure and dried under reduced pressure, thereby obtaining 3.80 g of a compound (C-2-11).

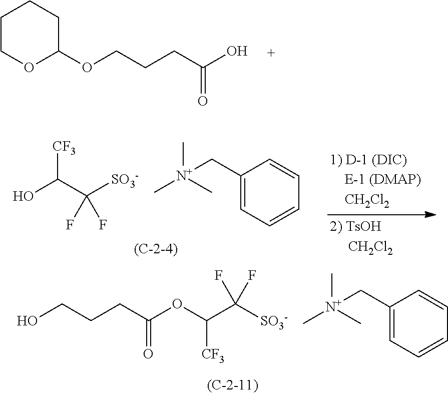

The raw materials, the intermediates (the compound (B0p) and the like), and the compounds finally obtained (the compound (b0′) and the like), which were used in the above-described method for producing a compound of each example are listed in Tables 5 to 8.

TABLE 5

| | | | | Condensing | | | |
|---|---|---|---|---|---|---|---|
| | Compound | Compound | Compound | agent/activating | | Compound | Compound |
| | (C1) | (C2) | (C3) | agent | Additive | (B0p) | (b0′) |
| Example 1a | C-1-1 | C-2-1 | C-3-1 | D-1 | E-1 | (B0p-1) | (b0-1) |
| Example 2a | C-1-2 | C-2-1 | C-3-1 | D-1 | E-1 | (B0p-2) | (b0-2) |
| Example 3a | C-1-3 | C-2-1 | C-3-1 | D-1 | E-1 | (B0p-3) | (b0-3) |
| Example 4a | C-1-4 | C-2-1 | C-3-1 | D-1 | E-1 | (B0p-4) | (b0-4) |
| Example 5a | C-1-5 | C-2-1 | C-3-1 | D-1 | E-1 | (B0p-5) | (b0-5) |
| Example 6a | C-1-6 | C-2-1 | C-3-1 | D-1 | E-1 | (B0p-6) | (b0-6) |
| Example 7a | C-1-7 | C-2-1 | C-3-1 | D-1 | E-1 | (B0p-7) | (b0-7) |
| Example 8a | C-1-8 | C-2-1 | C-3-1 | D-1 | E-1 | (B0p-8) | (b0-8) |
| Example 9a | C-1-9 | C-2-1 | C-3-1 | D-1 | E-1 | (B0p-9) | (b0-9) |
| Example 10a | C-1-10 | C-2-1 | C-3-1 | D-1 | E-1 | (B0p-10) | (b0-10) |
| Example 11a | C-1-11 | C-2-1 | C-3-1 | D-1 | E-1 | (B0p-11) | (b0-11) |
| Example 12a | C-1-12 | C-2-1 | C-3-1 | D-1 | E-1 | (B0p-12) | (b0-12) |
| Example 13a | C-1-13 | C-2-1 | C-3-1 | D-1 | E-1 | (B0p-13) | (b0-13) |

TABLE 6

| | | | | Condensing | | | |
|---|---|---|---|---|---|---|---|
| | Compound | Compound | Compound | agent/activating | | Compound | Compound |
| | (C1) | (C2) | (C3) | agent | Additive | (B0p) | (b0′) |
| Example 14a | C-1-12 | C-2-2 | C-3-1 | D-1 | E-1 | (B0p-14) | (b0-12) |
| Example 15a | C-1-12 | C-2-4 | C-3-1 | D-1 | E-1 | (B0p-15) | (b0-14) |
| Example 16a | C-1-12 | C-2-5 | C-3-1 | D-1 | E-1 | (B0p-16) | (b0-15) |
| Example 17a | C-1-2 | C-2-4 | C-3-1 | D-1 | E-1 | (B0p-17) | (b0-16) |
| Example 18a | C-1-7 | C-2-4 | C-3-1 | D-1 | E-1 | (B0p-18) | (b0-17) |
| Example 19a | C-1-9 | C-2-4 | C-3-1 | D-1 | E-1 | (B0p-19) | (b0-18) |
| Example 20a | C-1-12 | C-2-1 | C-3-2 | D-1 | E-1 | (B0p-12) | (b0-19) |
| Example 21a | C-1-12 | C-2-1 | C-3-3 | D-1 | E-1 | (B0p-12) | (b0-20) |
| Example 22a | C-1-12 | C-2-1 | C-3-4 | D-1 | E-1 | (B0p-12) | (b0-21) |
| Example 23a | C-1-12 | C-2-1 | C-3-5 | D-1 | E-1 | (B0p-12) | (b0-22) |
| Example 24a | C-1-12 | C-2-1 | C-3-6 | D-1 | E-1 | (B0p-12) | (b0-23) |
| Example 25a | C-1-12 | C-2-1 | C-3-1 | D-2 | E-1 | (B0p-12) | (b0-12) |
| Example 26a | C-1-12 | C-2-1 | C-3-1 | D-3 | E-1 | (B0p-12) | (b0-12) |

TABLE 7

| | | | | Condensing | | | |
|---|---|---|---|---|---|---|---|
| | Compound | Compound | Compound | agent/activating | | Compound | Compound |
| | (C1) | (C2) | (C3) | agent | Additive | (B0p) | (b0′) |
| Example 27a | C-1-12 | C-2-1 | C-3-1 | D-4 | — | (B0p-12) | (b0-12) |
| Example 28a | C-1-12 | C-2-1 | C-3-1 | D-5 | — | (B0p-12) | (b0-12) |
| Example 29a | C-1-12 | C-2-1 | C-3-1 | D-6 | E-1 | (B0p-12) | (b0-12) |
| Example 30a | C-1-12 | C-2-1 | C-3-1 | D-7 | E-1 | (B0p-12) | (b0-12) |
| Example 31a | C-1-14 | C-2-7 | C-3-1 | — | — | (B0p-20) | (b0-24) |
| Example 32a | C-1-12 | C-2-1 | C-3-1 | D-1 | E-2 | (B0p-12) | (b0-12) |
| Example 33a | C-1-12 | C-2-1 | C-3-1 | D-1 | E-3 | (B0p-12) | (b0-12) |
| Example 34a | C-1-12 | C-2-8 | C-3-1 | D-1 | E-1 | (B0p-21) | (b0-12) |
| Example 35a | C-1-12 | C-2-9 | C-3-1 | D-1 | E-1 | (B0p-22) | (b0-12) |
| Example 36a | C-1-12 | C-2-10 | C-3-1 | D-1 | E-1 | (B0p-23) | (b0-12) |
| Example 37a | C-1-12 | C-2-11 | C-3-1 | D-1 | E-1 | (B0p-24) | (b0-25) |
| Example 38a | C-1-12 | C-2-3 | C-3-1 | D-1 | E-1 | (B0p-25) | (b0-12) |
| Example 39a | C-1-12 | C-2-6 | C-3-1 | D-1 | E-1 | (B0p-26) | (b0-15) |

TABLE 8

| | | | | Condensing | | | |
|---|---|---|---|---|---|---|---|
| | Compound | | Compound | agent/activating | | Compound | Compound |
| | (C1) | Compound | (C3) | agent | Additive | (B1p) | (b0′) |
| Comparative | C-1-12 | X-2 | C-3-1 | D-1 | E-1 | (B0p-26) | (b0-15) |
| Example 1a | | | | | | | |
| Comparative | C-1-12 | X-3 | C-3-1 | D-1 | E-1 | (B0p-16) | (b0-15) |
| Example 2a | | | | | | | |
| Comparative | C-1-12 | X-1 | C-3-1 | D-1 | E-1 | (B1p-1) | (b0-12) |
| Example 3a | | | | | | | |

In Tables 5 to 8, each abbreviation has the following meaning.
C-1-1 to C-1-14: compounds (C-1-1) to (C-1-14) shown above
C-2-1 to C-2-11: compounds (C-2-1) to (C-2-11) shown above
X-1 to X-3: compounds (X-1) to (X-3) shown above
C-3-1 to C-3-6: compounds (C-3-1) to (C-3-6) shown above
D-1 to D-7: compounds D-1 to D-7 each represented by Chemical Formulae D-1 to D-7
E-1 to E-3: compounds E-1 to E-3 each represented by Chemical Formulae E-1 to E-3

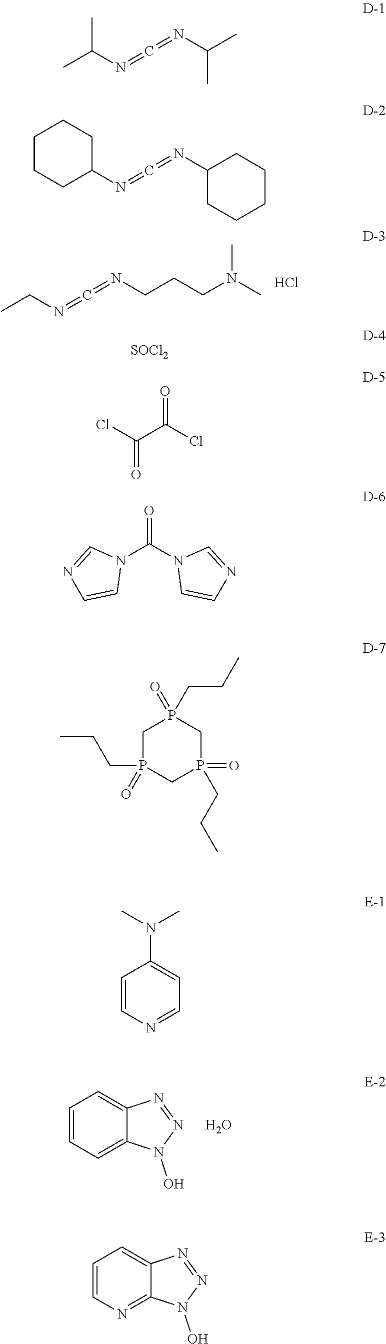

The yield in the method for producing a compound of each example (the yield in the step A, the yield in the step B, and the total yield (the yield in the step A x the yield in the step B)), the amounts of isomers determined by the following measuring method, and the remaining amounts of Na are listed in Tables 9 to 12. Further, the production method of the comparative examples does not include the step A or the step B, but the step corresponding to the step A of the examples is denoted as the step A, and the step corresponding to the step B of the examples is denoted as the step B for convenience.

### [Measurement of Amount of Isomers]

The amount of impurities (the amount of isomers) in the compound obtained by the method for producing a compound of each example was quantified by LC-MS. “n.d.” denotes that the amount was less than the detection limit.

### [Measurement of Remaining Amount of Na]

The remaining amount of Na in the compound obtained by the method for producing a compound of each example was quantified by ICP-MS.

TABLE 9

| | | | | | Remaining |
|---|---|---|---|---|---|
| | Step A | Step B | Total | Amount of | amount of |
| | yield | yield | yield | isomer | Na |
| Example 1a | 90% | 92% | 83% | n.d. | <10 ppb |
| Example 2a | 89% | 92% | 82% | n.d. | <10 ppb |
| Example 3a | 87% | 93% | 81% | n.d. | <10 ppb |
| Example 4a | 88% | 90% | 79% | n.d. | <10 ppb |
| Example 5a | 89% | 92% | 82% | n.d. | <10 ppb |
| Example 6a | 89% | 92% | 82% | n.d. | <10 ppb |
| Example 7a | 88% | 91% | 80% | n.d. | <10 ppb |
| Example 8a | 90% | 93% | 84% | n.d. | <10 ppb |
| Example 9a | 89% | 92% | 82% | n.d. | <10 ppb |
| Example 10a | 89% | 91% | 81% | n.d. | <10 ppb |
| Example 11a | 89% | 92% | 82% | n.d. | <10 ppb |
| Example 12a | 91% | 94% | 86% | n.d. | <10 ppb |
| Example 13a | 89% | 90% | 80% | n.d. | <10 ppb |

TABLE 10

| | | | | | Remaining |
|---|---|---|---|---|---|
| | Step A | Step B | Total | Amount of | amount of |
| | yield | yield | yield | isomer | Na |
| Example 14a | 88% | 93% | 82% | n.d. | <10 ppb |
| Example 15a | 85% | 94% | 80% | n.d. | <10 ppb |
| Example 16a | 89% | 92% | 82% | n.d. | <10 ppb |
| Example 17a | 90% | 91% | 82% | n.d. | <10 ppb |
| Example 18a | 88% | 92% | 81% | n.d. | <10 ppb |
| Example 19a | 89% | 92% | 82% | n.d. | <10 ppb |
| Example 20a | 89% | 92% | 82% | n.d. | <10 ppb |
| Example 21a | 85% | 94% | 80% | n.d. | <10 ppb |
| Example 22a | 89% | 92% | 82% | n.d. | <10 ppb |
| Example 23a | 85% | 94% | 80% | n.d. | <10 ppb |
| Example 24a | 89% | 91% | 81% | n.d. | <10 ppb |
| Example 25a | 87% | 93% | 81% | n.d. | <10 ppb |
| Example 26a | 87% | 93% | 81% | n.d. | <10 ppb |

TABLE 11

| | | | | | Remaining | |
|---|---|---|---|---|---|---|
| | Step A | Step B | Total | Amount of | amount of | |
| | yield | yield | yield | isomer | Na | |
| Example 27a | 87% | 93% | 81% | n.d. | <10 | ppb |
| Example 28a | 89% | 91% | 81% | n.d. | <10 | ppb |
| Example 29a | 90% | 90% | 81% | n.d. | <10 | ppb |
| Example 30a | 88% | 92% | 81% | n.d. | <10 | ppb |
| Example 31a | 87% | 93% | 81% | n.d. | <10 | ppb |
| Example 32a | 88% | 92% | 81% | n.d. | <10 | ppb |
| Example 33a | 88% | 92% | 81% | n.d. | <10 | ppb |
| Example 34a | 87% | 93% | 81% | n.d. | <10 | ppb |
| Example 35a | 88% | 91% | 80% | n.d. | <10 | ppb |
| Example 36a | 88% | 90% | 79% | n.d. | <10 | ppb |
| Example 37a | 88% | 90% | 79% | n.d. | <10 | ppb |
| Example 38a | 87% | 88% | 77% | n.d. | 200 | ppb |
| Example 39a | 88% | 88% | 77% | n.d. | 200 | ppb |

TABLE 12

| | | | | | Remaining | |
|---|---|---|---|---|---|---|
| | Step A | Step B | Total | Amount of | amount of | |
| | yield | yield | yield | isomer | Na | |
| Comparative | 60% | 90% | 54% | 5% | 200 | ppb |
| Example 1a | | | | | | |
| Comparative | 55% | 89% | 49% | n.d. | <10 | ppb |
| Example 2a | | | | | | |
| Comparative | 87% | 24% | 21% | n.d. | <10 | ppb |
| Example 3a | | | | | | |

As listed in Tables 9 to 12, it was confirmed that the target compounds with a higher yield were produced by the methods for producing a compound of Examples 1a to 39a as compared with the methods for producing a compound of Comparative Examples 1a to 3a.

In addition, among the examples, in the methods for producing a compound of Examples 1a to 37a, since an organic ammonium salt was used as the compound (C2), the remaining amount of Na could be further reduced.

In the method for producing a compound of Comparative Example 1a, since the compound (X-2) was used in place of the compound (C2), and the terminal was sulfonated after the condensation reaction was performed on the compound (C1-1-12) and the compound (X-2), a large number of isomers were generated and the yield in the step A was low.

In the method for producing a compound of Comparative Example 2a, since the compound (X-3) having a chlorine atom at the terminal was used in place of the compound (C2), the yield in the step A was low.

In the method for producing a compound of Comparative Example 3a, since the compound (X-1) having a cation moiety with a high Log P value (Log P value: 7.81) and having relatively high hydrophobicity was used in place of the compound (C2), the salt exchange reaction did not proceed sufficiently, and the yield in the step B was low.

While preferred embodiments of the invention have been described and illustrated above, it should be understood that these are exemplary of the invention and are not to be considered as limiting. Additions, omissions, substitutions, and other modifications can be made without departing from the scope of the invention. Accordingly, the present invention is not to be considered as being limited by the foregoing description and is only limited by the scope of the appended claims.

What is claimed is:

1. A resist composition which generates an acid upon light exposure and whose solubility in a developing solution is changed due to an action of the acid, the resist composition comprising:
a resin component (A1) whose solubility in a developing solution is changed due to the action of the acid; and
an acid generator component (B) which generates an acid upon light exposure,
wherein the acid generator component (B) contains a compound (B0) represented by General Formula (b0): 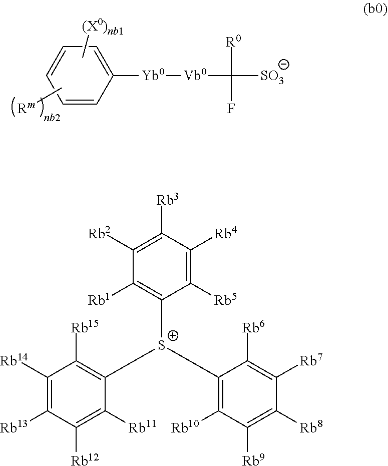
wherein X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, provided that 1≤nb1+nb2≤5 is satisfied, Yb⁰ represents a divalent linking group or a single bond, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represents a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group, 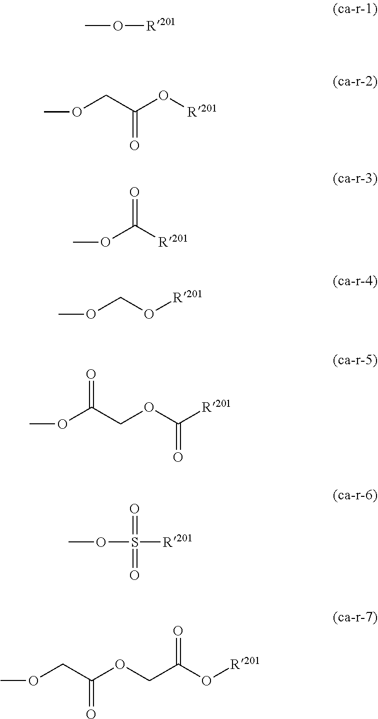
wherein each R′²⁰¹ independently represents a hydrogen atom, a cyclic group which may have a substituent, a chain alkyl group which may have a substituent, or a chain alkenyl group which may have a substituent.

2. The resist composition according to claim 1, wherein the compound (B0) includes a compound (B01) represented by General Formula (b0-1): 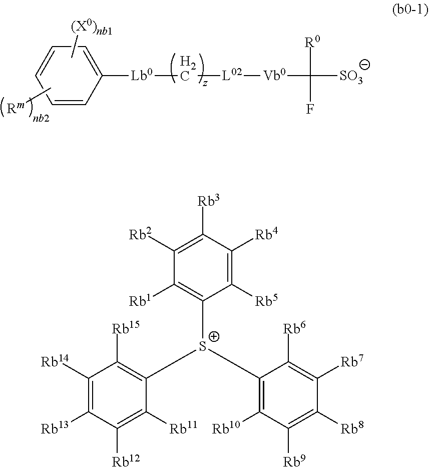
wherein X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, provided that 1≤nb1+nb2≤5 is satisfied, L⁰¹ and L⁰² each independently represents a single bond, an alkylene group, —O—, —CO—, —OCO—, —COO—, —SO₂—, —C(R^{b})═N—, or —CON(R^{b})—, R^{b} represents a hydrogen atom or an alkyl group, z represents an integer of 0 to 10, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represents a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group, 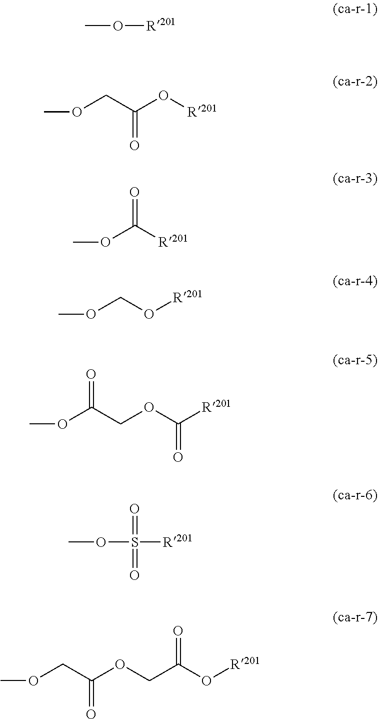
wherein each R′²⁰¹ independently represents a hydrogen atom, a cyclic group which may have a substituent, a chain alkyl group which may have a substituent, or a chain alkenyl group which may have a substituent.

3. The resist composition according to claim 1, further comprising:
a base component (D) which traps an acid generated upon light exposure,
wherein the base component (D) contains a compound represented by General Formula (d1-1): 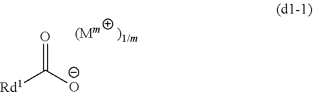
wherein Rd¹ represents a cyclic group which may have a substituent, a chain alkyl group which may have a substituent, or a chain alkenyl group which may have a substituent, m represents an integer of 1 or greater, and each M^{m+} independently represents an m-valent organic cation.

4. A method of forming a resist pattern, comprising:
forming a resist film on a support using the resist composition according to claim 1;
exposing the resist film to light; and
developing the resist film exposed to light to form a resist pattern.

5. The resist pattern formation method according to claim 4,
wherein the resist film is exposed to an extreme ultraviolet (-EU-V) ray or an electron beam in exposing the resist film to light.

6. A compound represented by General Formula (b0): 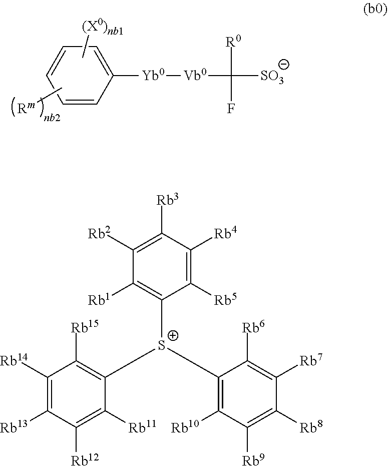
wherein X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, provided that 1≤nb1+nb2≤5 is satisfied, Yb⁰ represents a divalent linking group or a single bond, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently r-e-present represents a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group, 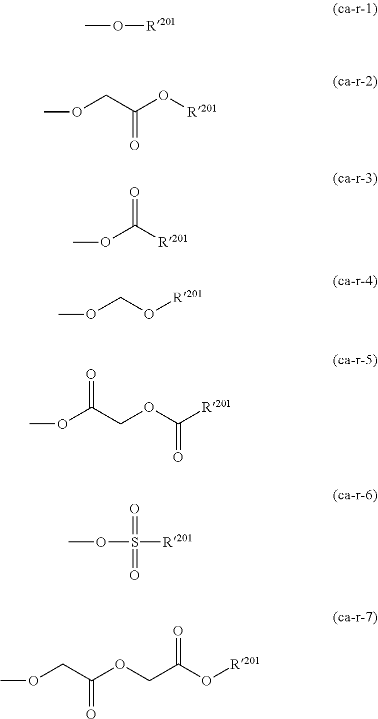
wherein each R′²⁰¹ independently represents a hydrogen atom, a cyclic group which may have a substituent, a chain alkyl group which may have a substituent, or a chain alkenyl group which may have a substituent.

7. The compound according to claim 6, wherein the compound is represented by General Formula (b0-1): 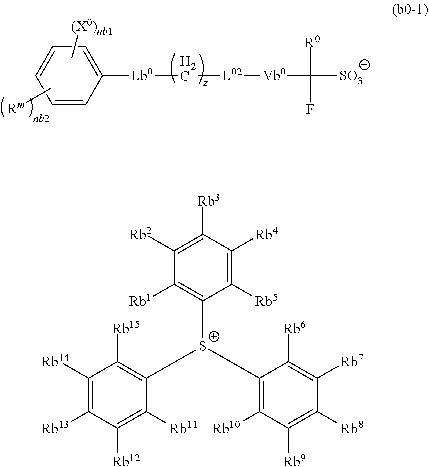
wherein X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, provided that 1≤nb1+nb2≤5 is satisfied, L⁰¹ and L⁰² each independently represents a single bond, an alkylene group, —O—, —CO—, —OCO—, —COO—, —SO₂—, —C(Rb)═N—, or —CON(Rb)—, R^{b} represents a hydrogen atom or an alkyl group, z represents an integer of 0 to 10, Vb⁰ represents a single bond, an alkylene group, or a fluorinated alkylene group, R⁰ represents a hydrogen atom, a fluorinated alkyl group having 1 to 5 carbon atoms, or a fluorine atom, Rb¹ to Rb¹⁵ each independently represents a hydrogen atom, an alkyl group, a halogen atom, a halogenated alkyl group, a carbonyl group, a cyano group, an amino group, an aryl group, or a group represented by any of General Formulae (ca-r-1) to (ca-r-7), and Rb¹⁰ and Rb¹¹ may be bonded to each other to form a ring with a sulfur atom in the formula, where at least two of Rb¹ to Rb⁵ represent a fluorine atom or at least one of Rb¹ to Rb⁵ represents a perfluoroalkyl group, 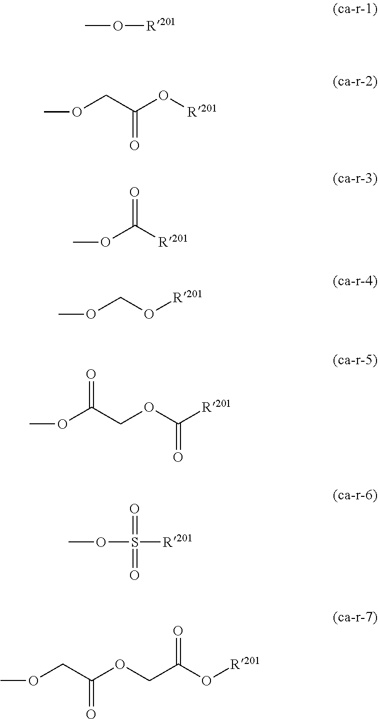
wherein each R′²⁰¹ independently represents a hydrogen atom, a cyclic group which may have a substituent, a chain alkyl group which may have a substituent, or a chain alkenyl group which may have a substituent.

8. An acid generator comprising the compound according to claim 6.

9. A method for producing a compound, comprising:
performing a condensation reaction on a compound represented by General Formula (C-1) and a compound represented by General Formula (C-2) to obtain a compound (B0p) represented by General Formula (b0-p); and
performing an ion exchange reaction on the compound (B0p) and a compound represented by General Formula (C-3) to obtain a compound (b0′) represented by General Formula (b0′): 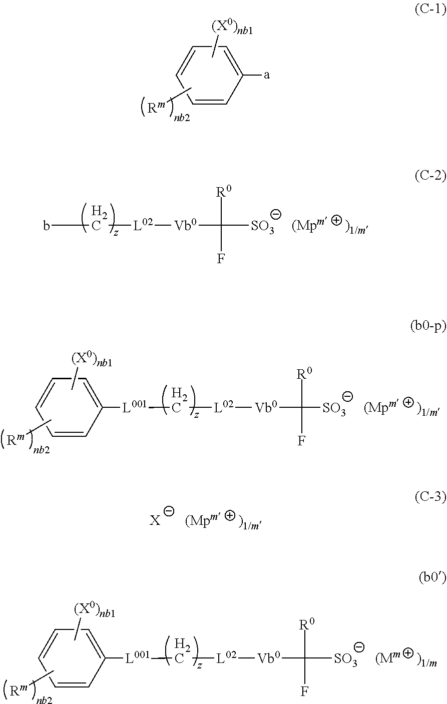
wherein X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, provided that 1≤nb11+nb2≤5 is satisfied, one of a and b represents a hydroxy group, and the other represents a carboxy group, z represents an integer of 0 to 10, L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a})—, or —C(═O)—N(R^{a})—, each R^{a} independently represents a hydrogen atom or an alkyl group, Vb⁰ represents a single bond, an alkylene group, or a fluorinated alkylene group, R⁰ represents a hydrogen atom, a fluorinated alkyl group having 1 to 5 carbon atoms, or a fluorine atom, Mp^{m′+} represents a metal cation or an organic ammonium cation having a Log P of 4.8 or less, m′ represents an integer of 1 or greater, L⁰⁰ 1 represents an ester bond [—C(═O)—O— or —O—C(═O)—], X-represents a counter anion, M^{m+} represents an m-valent organic cation, and m represents an integer of 1 or greater.

10. An intermediate which is used in the method for producing a compound according to claim 9,
wherein the intermediate is represented by General Formula (b0p): 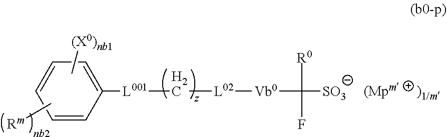
wherein X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, provided that 1≤nb1+nb2≤5 is satisfied, L° ° ¹ represents an ester bond [—C(═O)—O— or —O—C(═O)—], z represents an integer of 0 to 10, L⁰² represents a single bond, an alkylene group, —O—, —CO—, —OCO—, —COO—, —SO₂—, —N(R^{a})—C(═O)—, —N(R^{a})—, —C(R^{a})(R^{a})—N(R^{a})—, —C(R^{a})(N(R^{a})(R^{a}))—, or —C(═O)—N(R^{a})—, each R^{a} independently represents a hydrogen atom or an alkyl group, Vb⁰ represents a single bond, an alkylene group, or a fluorinated alkylene group, R⁰ represents a hydrogen atom, a fluorinated alkyl group having 1 to 5 carbon atoms, or a fluorine atom, M^{m′+} represents a metal cation or an organic ammonium cation having a Log P of 4.8 or less, and m′ represents an integer of 1 or greater.

11. A compound which is represented by General Formula (b0-p-1): 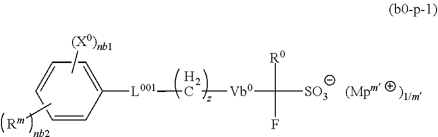
wherein X⁰ represents a bromine atom or an iodine atom, R^{m} represents a hydroxy group, an alkyl group, a fluorine atom, or a chlorine atom, nb1 represents an integer of 1 to 5, and nb2 represents an integer of 0 to 4, provided that 1≤nb1+nb2≤5 is satisfied, L° ° ¹ represents an ester bond [—C(═O)—O— or —O—C(═O)—], z represents an integer of 0 to 10, Vb⁰ represents a single bond, an alkylene group, or a fluorinated alkylene group, R⁰ represents a hydrogen atom, a fluorinated alkyl group having 1 to 5 carbon atoms, or a fluorine atom, M^{m′+} represents a metal cation or an organic ammonium cation having a Log P of 4.8 or less, and m′ represents an integer of 1 or greater.
